(19) United States
(12) Patent Application Publication
(10) Pub. No.: US 2019/0099336 A1
(43) Pub. Date: Apr. 4, 2019

(54) Compositions, Methods and Uses for Improving a Skin or Hair Region

(71) Applicant: Collidion, Inc., Petaluma, CA (US)

(72) Inventor: Emma Alimi, Petaluma, CA (US)

(73) Assignee: Collidion, Inc., Petaluma, CA (US)

(21) Appl. No.: 16/151,294

(22) Filed: Oct. 3, 2018

Related U.S. Application Data
(60) Provisional application No. 62/567,730, filed on Oct. 3, 2017.
(60) Provisional application No. 62/572,445, filed on Oct. 14, 2017.

(51) Int. Cl. A61K 8/20; A61K 8/891; A61K 8/895; A61K 8/25; A61Q 19/00; A61Q 5/00

(52) U.S. Cl. CPC A61K 8/20; A61K 8/891; A61K 8/895; A61K 8/25; A61K 2800/5922; A61Q 5/00; A61K 2800/88; A61K 2800/20; A61K 2800/594; A61Q 19/00

(57) ABSTRACT

The present specification discloses a composition comprising hypochlorous acid or free available chlorine and one or more silicones, and methods to improve and/or support the appearance, beauty and/or health of a skin region or a hair region in an individual by topically applying a disclosed composition.

This application claims the benefit of priority and the filing date pursuant to 35 U.S.C. § 119(e) to U.S. Provisional Patent Application 62/572,445, filed Oct. 14, 2017, and U.S. Provisional Patent Application 62/567,730, filed Oct. 3, 2017, the entire content of each of which is hereby incorporated by reference in its entirety.

The global personal care industry broadly refers to an extremely diversified group of products and services used for personal hygiene individual wellbeing, and beauty care (cosmetic) to enhance, support or alter the appearance of the body or body odor. Personal care products may be classified on the basis of application such as hair care, skin care, oral care, color (make-up), fragrances and toiletries; and by type such as organic, inorganic and herbal. Personal care products include toothpaste, fragrances, mouthwashes, antiperspirants/deodorants, skin care products, hair care products, hair dyeing products, make-up products, sanitary products, sun care products, and products for nail care, foot care, bathing and shaving. Apart from products, this industry also includes equipment, supplies, and furniture for beauty salons, health spas, and fitness centers. In addition, the personal care industry overlaps with a number of sectors including health care, disinfectant/cleaner, chemical and petroleum due to the raw materials for manufacture of personal care products coming from these industries.

The global market for personal care products and services is expected to reach $630 billion in 2017, with a compound annual growth rate of 3.4% over the next five years. This global market is usually divided into four regions: North America, Europe, Asia Pacific, and the Rest of the World. Currently North America is the largest market for personal care product and service consumption, followed closely by Europe, due to the high sophistication and awareness for personal hygiene among consumers in these regions. However, Asia Pacific is expected to witness the fastest growth in the personal care industry over the next five years, owing to increasing consumer disposable income and growing awareness for personal hygiene in the region. Emerging economies such as Brazil and Mexico are also predicted to have high growth potential due to the rise in disposable incomes.

As consumer preferences in the human cosmetic market shift from mass-targeted products to premium targeted solutions this has led to a demand for the development of more specialized and sophisticated products that aim to achieve higher performance and enhanced benefits. One example seen in consumer trends is the increased demand for cosmetics with combined benefits and/or features from different product categories. As such, there is an increasing global desire in the personal care market for specialized formulations that compound an immediate altered effect/s of a make-up (cover imperfection, smooth uneven texture, reduce the appearance of wrinkles/foundations, blemish or wrinkle concealer creams, lipsticks, makeup primers, pigments) and deliver a skincare benefit/s (hydration, rejuvenate, bring glow, vitality/moisturizers, toners, cleansers, sunscreens, anti-aging, targeted serums, gels). This growing category of specialized personal care products has added millions of dollars to global cosmetics markets, with wrinkle concealer creams and makeup primers products alone contributing over $490 million to the industry in 2015, growing 58 percent over two years.

Another aspect of the growth seen in the global market for personal care products and services is attributable to veterinary and pet care uses. As affluence and the associated increase disposable income rises, consumers purchase personal care products not only for themselves, but also their pets. For example, over 65% of households in the United States own at least one pet. In 2016, the pet industry generated revenues of over $60 billion, with about $5 billion spent on pet services such as grooming and boarding.

Thus, there is a great world-wide desire for personal care products for both human and animal uses. This desire has also led to the demand of consumers for more specialized and sophisticated products that achieve better performance or other enhanced benefits. As such, there is a continuing need to develop new personal care products.

### SUMMARY

Aspects of the present specification disclose a composition comprising hypochlorous acid or free available chlorine. The disclosed composition may further comprise and one or more silicones and/or one or more rheology agents and one or more carriers as well as optional additional ingredients.

Aspects of the present specification disclose a composition comprising hypochlorous acid or free available chlorine and one or more silicones. The disclosed composition may further comprise one or more rheology agents and one or more carriers as well as optional additional ingredients.

Other aspects of the present specification disclose a method to improve and/or support the appearance, beauty and/or health of a skin region in an individual. The disclosed method comprising topically applying a composition disclosed herein, wherein application of the composition restores, maintains and/or improves an attribute or a characteristic of the skin region, thereby improving and/or supporting the appearance, beauty and/or health of the skin region.

Other aspects of the present specification disclose a method to improve and/or support the appearance, beauty and/or health of a hair region in an individual. The disclosed method comprising topically applying a composition disclosed herein, wherein application of the composition restores, maintains and/or improves an attribute or a characteristic of the hair region, thereby improving and/or supporting the appearance, beauty and/or health of the hair region.

Other aspects of the present specification disclose a composition disclosed herein for use to improve and/or support the appearance, beauty and/or health of a skin region in an individual.

Other aspects of the present specification disclose a composition disclosed herein for use to improve and/or support the appearance, beauty and/or health of a hair region in an individual.

Other aspects of the present specification disclose a use of a disclosed composition to improve and/or support the appearance, beauty and/or health of a skin region in an individual.

Other aspects of the present specification disclose a use of a disclosed to improve and/or support the appearance, beauty and/or health of a hair region in an individual.

Other aspects of the present specification disclose a use of a disclosed composition in the manufacture of a medicament to improve and/or support the appearance, beauty and/or health of a skin region in an individual.

Other aspects of the present specification disclose a use of a disclosed composition in the manufacture of a medicament to improve and/or support the appearance, beauty and/or health of a hair region in an individual.

### DETAILED DESCRIPTION

The skin is the outer covering of a mammalian body and guards the underlying muscles, bones, ligaments and internal organs. Typically, the largest organ of the integumentary system, skin is composed of three primary layers: the epidermis, the dermis and the hypodermis. The epidermis is the outermost epithelial layer of the skin and forms the waterproof, protective wrap over the body's surface which also serves as a barrier to infection. The dermis is an epithelial layer of skin beneath the epidermis and serves to cushions the body from stress and strain. The hypodermis lies below the dermis and comprises loose connective tissue, adipose tissue and elastin. Although not composed of epithelial tissue, and thus not part of the skin, the hypodermis attaches the skin to underlying bone and muscle as well as supplying it with blood vessels and nerves.

Mammalian skin changes drastically with age, losing volume, elasticity, firmness and resiliency due to the thinning of the epithelial layers of the epidermis and dermis, and degeneration of the underlying fat and connective tissue of the hypodermis. These atrophic changes affect all skin areas, including facial skin, neck skin, chest skin, breast skin, hand skin, leg skin, and feet skin. Symptoms associated with skin aging include, e.g., wrinkles, fine lines, thinning skin, sagging skin, sinking skin, skin dryness, and skin itchiness.

Hair is composed of two structures: the hair shaft, the hard protein filament that extends above the skin's surface, and the hair follicle, the organ located in the dermis that maintains stem cells. The hair shaft is primary composed of keratin that is organized into three layers called the medulla, cortex and cuticle. The medulla is the inner layer is not necessarily present in all hair types. The next keratin layer is the cortex, the intermediate layer that makes up the majority of the hair shaft and provides the hair's strength. Pigment cells, or melanocytes, are distributed throughout the cortex and medulla giving the hair its characteristic color. The outer layer is the cuticle, which is formed by tightly packed translucent scales in an overlapping structure similar to roof shingles and is continuous with the root sheath. The cuticle protects the cortex fibers from damage and is responsible for the surface properties of hair, such as shine and smoothness. The hair follicle comprises the bulb, which contains the cells that produce the hair shaft, the oil producing sebaceous gland which lubricates the hair and the arrector pili muscles, which are responsible for causing hairs to stand up.

Mammalian hair is constantly undergoing injury through environmental exposure or damaging processes including physical and chemical. Environmental exposure that damage the hair includes solar radiation (irradiation with UV light), humidity, pollution and salt and chlorinated water. Physical processes causing hair damage include, brushing, combing and heating by using a styling appliance like a blow dryer, flat iron, or curling iron. Chemical processes include shampooing, weaving, perming, straightening, relaxing, highlighting, streaking, coloring, dyeing and bleaching. Hair damaged by these processes losses its natural suppleness, luster and manageability becoming rough, dry and brittle as well as exhibiting undesirable color change, hair loss and loss of shine. Such damage not only makes hair more susceptible to further damage, including breakage and frayed or split ends, but also makes hair more difficult to manage, comb and/or disentangle.

The present specification discloses personal care products designed to address the undesirable effects seen in skin during aging as well as those unwanted effects observed in hair damaged by exposure to the environment or physical and/or chemical processes. A composition disclosed herein is intended to beautifying, promoting attractiveness, or altering the appearance of skin and hair. In an aspect of this embodiment, the present specification discloses an anti-aging composition designed to increase the volume, elasticity, firmness and/or resiliency of skin. In another aspect of this embodiment, the present specification discloses a hair smoothing composition designed to increase the hydrophobicity and restore the natural smooth, lubricious feel of hair. In addition, a composition disclosed herein is may also provide a therapeutic or clinical effect in that use could reduce one or more symptoms associated with a skin or hair condition, such as, e.g., reduce itching, reduce pain, or promote healing of a skin abrasion.

A composition disclosed herein may comprise hypochlorous acid. A weak acid, the chemical formula of hypochlorous acid is HOCl, while its molecular formula is written as HCIO. As shown in formula I, hypochlorous acid is a simple molecule with the central oxygen connected to chlorine and hydrogen atoms through single bonds and has molar mass is 52.46 g/mol.

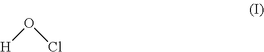

Hypochlorous acid is a colorless solution, and its exact physical properties are variable, depending on the concentration of hypochlorous in solution. Hypochlorous acid reacts with bases to form salts called hypochlorites. For example, sodium hypochlorite (NaOCl), the active ingredient in bleach, is formed by reacting hypochlorous acid with sodium hydroxide. Hypochlorous acid also readily reacts with a variety of organic molecules and biomolecules.

The hypochlorous acid solution can be produced, e.g., by dissolving chlorine in water, hydrolysis of chlorine gas, electrolysis of a salt solution or acidification of hypochloride. For example, stable hypochlorous salts, such as, e.g., alkali metal hypochlorites like sodium hypochlorite, calcium hypochlorite, potassium hypochlorite, lithium hypochlorite and magnesium hypochlorite, can be obtained by dissolving chlorine gas into an aqueous alkali metal hydroxide solution, like a sodium hydroxide solution, a calcium hydroxide solution, a potassium hydroxide solution, a lithium hydroxide solution, or a magnesium hydroxide solution. Hypochlorous acid can also be prepared by dissolving dichlorine monoxide in water. As another example, hypochlorous acid can also be produced by electrolytically treating a saline solution. In one method, an electrical current is applied to a three-compartment cell comprising a cathode chamber, an anode chamber, and a central saline solution chamber interposed between the other two chambers where each chamber is separated by a semi-permeable membrane. During electrolysis, sodium chloride (NaCl) dissociates into negatively charged chloride (Cl⁻) and positively charged sodium (Na⁺). At the same time, water dissociates into hydroxide (OH⁻) and hydrogen (H⁺) ions are formed. The negatively charged chloride (Cl⁻) and hydroxide (OH⁻) ions move to the anode to lose electrons and form hypochlorous acid (HOCl) as we as hypochlorite ions (OCl⁻) and oxygen (O₂) and chlorine (Cl₂) gases. The reductive water comprising the hypochlorous acid is then dispensed into a collection chamber for subsequent use. Methods to produce hypochlorous acid are described in, e.g., U.S. Pat. Nos. 3,914,397, 4,190,638, 4,908,215, 5,322,677, 6,426,066, 7,323,118, 8,062,500, 8,945,630, 9,168,318, and 9,486,479, each of which is hereby incorporated by reference in its entirety.

In an embodiment, a composition disclosed herein comprises an amount of hypochlorous acid that provides a desired beneficial effect to a beauty composition, an anti-aging composition and/or a hair smoothing composition disclosed herein. In aspects of this embodiment, a composition disclosed herein comprises hypochlorous acid in an amount of, e.g., 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition. In other aspects of this embodiment, a composition disclosed herein comprises hypochlorous acid in an amount of, e.g., at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition. In yet other aspects of this embodiment, a composition disclosed herein comprises hypochlorous acid in an amount of, e.g., at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition. In still other aspects of this embodiment, a composition disclosed herein comprises hypochlorous acid in an amount of from, e.g., about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, or about 0.002% to about 0.01% by weight of the composition.

In aspects of this embodiment, a composition disclosed herein comprises hypochlorous acid in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm. In other aspects of this embodiment, a composition disclosed herein comprises hypochlorous acid in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm. In yet other aspects of this embodiment, a composition disclosed herein comprises hypochlorous add in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm.

In still other aspects of this embodiment, a composition disclosed herein comprises hypochlorous acid in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises hypochlorous acid in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

The concentration of hypochlorous acid in solution may be described as free available chlorine in parts per million. Hypochlorous acid is in equilibrium with hypochlorite ions (OCl and dissolved chlorine gas (Cl₂). The extent of the equilibrium is determined predominately by the pH of the solution. Temperature also impacts the ratio of the free chlorine component. Therefore, both FAC and pH need to be known to understand the amount of chlorine present as hypochlorous acid. In general, when the pH range is about 4.0 to about 5.6, approximately 100% of the available chlorine is present as HOCl. As the pH is lowered below about 4, there is an increase in dissolved chlorine gas (Cl₂). Thus, at a pH of about 3, about 90% of the available chlorine is present as hypochlorous acid, at a pH of about 2, about 75% of the available chlorine is present as hypochlorous acid, at a pH of about 1.5, about 50% of the available chlorine is present as hypochlorous acid, while at a pH of about 1, about 25% of the available chlorine is present as hypochlorous acid. As the pH is increase above about 5.6, there is an increase in hypochlorite ions (OCl⁻). Thus, at a pH a pH of about 6.5, about 90% of the available chlorine is present as hypochlorous acid, at a pH of about 7, about 75% of the available chlorine is present as hypochlorous acid, at a pH of about 7.5, about 50% of the available chlorine is present as hypochlorous acid, while at a pH of about 8, about 25% of the available chlorine is present as hypochlorous acid.

The chlorine amount may be measured by methods known in the art, such as the DPD colorimeter method (Lamotte Company, Chestertown, Md.) or other known methods established by the Environmental Protection Agency. In the DPD colorimeter method, a yellow color is formed by the reaction of free chlorine With N,N-diethyl-p-phenylenediamine (DPD) and the intensity is measured With a calibrated calorimeter that provides the output in parts per million. Further addition of potassium iodide turns the solution a pink color to provide the total chlorine value.

In aspects of this embodiment, a composition disclosed herein comprises free available chlorine in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm. In other aspects of this embodiment, a composition disclosed herein comprises free available chlorine in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm. In yet other aspects of this embodiment, a composition disclosed herein comprises free available chlorine in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm.

In still other aspects of this embodiment, a composition disclosed herein comprises free available chlorine in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises free available chlorine in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In one embodiment, a composition disclosed herein does not comprise ozone. In another embodiment, a composition disclosed herein can comprise ozone.

In one embodiment, a composition disclosed herein does not comprise hydrogen peroxide. In another embodiment, a composition disclosed herein can comprise hydrogen peroxide.

In one embodiment, a composition disclosed herein does not comprise dissolved inorganic carbon, which can form a bicarbonate or carbonate of an alkali or alkaline earth metal. In another embodiment, a composition disclosed herein can comprise dissolved inorganic carbon, which can form a bicarbonate or carbonate of an alkali or alkaline earth metal.

In one embodiment, a composition disclosed herein does not comprise sodium dihydrogen phosphate. In another embodiment, a composition disclosed herein can comprise sodium dihydrogen phosphate.

A composition disclosed herein may comprise a silicone. A silicone includes any member of a family of polymeric products whose molecular backbone is made up of alternating silicon and oxygen atoms with side groups attached to the silicon atoms. By varying the —Si—O— chain lengths, side groups, and crosslinking, a silicone can be synthesized into a wide variety of materials. They can vary in consistency from liquid to gel to solids. A silicone is highly hydrophobic, is known as an occlusive agent, and has properties that provide a variety of benefits and make them particularly advantageous in personal care products, including improved slip, reduced tack, enhanced emolliency, reduced transepidermal water loss, enhanced hydrated appearance, increased moisturizing characteristics, increased sebum and/or oil absorption, as well as improving the sensory and tactile feel and experience of the formulation, such as, e.g., cool touch, soft powdery finish, improved skin silkiness, improved skin smoothness, no or reduced stinging or burning sensation, no or reduced pungent (non-alcoholic) smell. In addition, silicones produce uniform thin films that are hydrophobic to create a barrier that prevents skin dehydration, and also produce compositions that possess a low viscosity. The low viscosity property allows higher loadings of active ingredients in a composition disclosed herein without the deleterious effects normally associated with high viscosity products. A silicone disclosed herein is also an excellent dispersing and spreading agent, is generally transparent to white in color, low in odor, and are resistant to chemical and oxidative attack. In addition, a silicone does not convey an oiliness or greasiness, providing a “smooth and silky” skin feel, wrinkle-masking properties, such as, e.g., increasing skin hydration, enhancing skin smoothness, reducing pore size, and masking skin imperfections, that is highly desirable in an anti-aging composition or hair smoothing composition disclosed herein. When incorporated into a hair smoothing composition disclosed herein, a silicone can provide benefits to hair health by reducing combing force, reducing drying time, protecting hair color, protecting hair from heat, strengthening hair, promoting hair shine and increasing hair hydrophobicity, thereby providing a smooth, soft feel to the hair, making the hair look full and shiny, and facilitating the combing of wet or dry hair.

A silicone disclosed herein includes any linear, branched or cyclic silicone or silicone derivative. In addition, a silicone disclosed herein can be classified as a non-volatile silicone, a volatile silicone, and a silicone surfactant. A non-volatile silicone includes, without limitation, a polysiloxane, a polysiloxane copolymer, a polyalkyl siloxane, a polyaryl siloxane, a polyalkylaryl siloxane, a polyether siloxane, a poly(alkylarylsiloxane)-(ethersiloxane) copolymer, a polydimethylsiloxane, a polydiphenylsiloxane, a poly(dimethylsiloxane)-(diphenylsiloxane) copolymer, a phenyl silicone, and a polysilicate siloxane copolymer. A volatile silicone includes, without limitation, a cyclomethicone. A silicone includes, without limitation, cyclomethicone, dimethicone, dimethicone crosspolymer, dimethicone copolyol alkyl alcohol, dimethiconol, trisiloxane, alkyl dimethicone, alkyl methicone, alkyl trimethyl silane, phenyl trimethicone and cyclopentasiloxane. Other silicones are described in, e.g., U.S. Pat. Nos. 7,700,530 and 8,840,911, each of which is incorporated by reference in its entirety.

Commercially available silicones include, without limitation, the dimethicone DOW CORNING 200 (Dow Corning Corporation, Midland, Mich.), the amodimethicomes DOW CORNING 2-8566 Amino Fluid (Dow Corning Corporation, Midland, Mich.), the dimethiconols E-2171 and S-701 (Siltech), the dimethicone crosspolymers DOW CORNING 9040, DOW CORNING 9041, DOW CORNING 9045, DOW CORNING 9546, and DOW CORNING 9701 (Dow Corning Corporation, Midland, Mich.), and the trisiloxane DOW CORNING 200 Fluid, 1 cSt (Dow Corning Corporation, Midland, Mich.), DC 345 (Dow Corning Corporation, Midland, Mich.), the cetyl dimethicone, such as ABIL Wax 9801, ABIL Wax 9814, or ABIL Wax 9840 (Evonik, Hopewell, Va.), and the cyclomethicones DOW CORNING 244 Fluid, DOW CORNING 245 Fluid, DOW CORNING 344 Fluid and DOW CORNING 345 Fluid (Dow Corning Corporation, Midland, Mich.), and SILICONE SF-1173 and SILICONE SF-1202 (Momentive Performance Materials, Wilton, Conn.).

A silicone disclosed herein includes a silicone amine and an anionic silicone. A silicone amine useful in a composition disclosed herein includes, without limitation, an amodimethicone, a trimethylsilylamodimethicone, a trimethylsilylamodimethicone, a phenyltrimethicone, an aminopropyl phenyltrimethicone, a trimethyloxysilane, a triethyoxysilane, a tearoxytrimethylsilane, an aminopropyl triethyoxysilane, an alkyl modified amino silicone, an amino silane, a polyethylene glycol based amodimethicone, a bis-isobutyl PEG-14/amodimethicone copolymer, a bis-isobutyl PEG-15/amodimethicone copolymer, and a bis-isobutyl PEG/PPG-20/35/amodimethicone copolymer. Non-limiting examples of a polyethylene glycol based amodimethicone includes a PEG-7 amodimethicone and a PEG-8 amodimethicone. Non-limiting examples of a modified aminosiloxane includes a Trideceth-9 PG-amodimethicone.

In an embodiment, a composition disclosed herein does not include amodimethicone.

An anionic silicone useful in a composition disclosed herein includes, without limitation, a polydimethylsiloxane, a silicone carboxylate, a silicone phosphate, a silicone sulfate, a silicone sulfosuccinate, a silicone sulfonate and a siloxane. Non-limiting examples of a polydimethylsiloxane includes a dimethicone, a vinyl dimethicone, a dimethicone/vinyl dimethicone copolymer, a dimethicone/vinyl dimethicone crosspolymer and a cyclodimethicone. Non-limiting examples of a silicone carboxylate includes a dimethicone PEG-7 phthalate and a dimethicone PEG-7 succinate. Non-limiting examples of a silicone phosphate includes a dimethicone PEG-7 phosphate, a dimethicone PEG-8 phosphate, a dimethicone PEG-10 phosphate, a potassium dimethicone PEG-7 panthenyl phosphate, a dimethicone PEG/PPG-7/4 phosphate and a dimethicone PEG/PPG-12/4 phosphate. Non-limiting examples of a siloxane include a polysilicone-11, a polysilicone-15, a silica silylate, a silica dimethyl silylate, a trimethylsiloxysilicate, a trifluoropropyldimethyl/trimethylsiloxysilicate and an alkyldimethylsilyl polypropylsilsesquioxane. Non-limiting examples of a cyclodimethicone includes hexamethylcyclotrisiloxane, octamethylcyclotetrasiloxane, decamethylcyclopentasiloxane and dodecamethylcyclohexasiloxane.

An silicone surfactant useful in a composition disclosed herein includes, without limitation, disodium PEG-12 dimethicone sulfosuccinate, PEG-12 dimethicone, PEG-10 dimethicone, PEG-8 dimethicone, PEG-7 dimethicone, PEG-7 dimethicone succinate, PEG-20/PPG-6 dimethicone, PEG-14/PPG-4 dimethicone, PEG-4/PPG-12 dimethicone, PEG-20/PPG-20 dimethicone, dimethylsiloxane and methyl(polyoxyethylene)siloxane copolymers, dimethylsiloxane and methyl(polyoxyethylene)siloxane and methyl(polyoxypropyl)siloxane copolymers, PEG-8 trisiloxane PEG-8/PPG-26 dimethicone, PEG-20/PPG-23 dimethicone, PEG-20/PPG-15 dimethicone, PEG-23/PPG-6 dimethicone, PEG-17 dimethicone, PPG-12 dimethicone, Quaternium-80, Quaternium 86, PEG-8 Distearmonium Chloride PG-Dimethicone, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-16/Glycidoxy Dimethicone Crosspolymer, Silicone Quaternium-17, Silicone Quaternium-18, Silicone Quaternium-19, Silicone Quaternium-20, Silicone Quaternium-21, Dimethicone Hydroxypropyl Trimonium Chloride, Hydroxyethyl Acetomonium PG-Dimethicone, Stearalkonium Dimethicone PEG-8 Phthalate, and Steardimonium Hydroxypropyl Panthenyl PEG-7 Dimethicone Phosphate Chloride.

In one embodiment, a single silicone is present in a composition disclosed herein. In another embodiment, a plurality of silicones is present in a composition disclosed herein. In aspects of this embodiment, a composition disclosed herein comprises, e.g., one or more silicones, two or more silicones, three or more silicones, four or more silicones or five or more silicones. In other aspects of this embodiment, a composition disclosed herein comprises, e.g., only one silicone, at most two silicones, at most three silicones, at most four silicones, or at most five silicones. In yet other aspects of this embodiment, a composition disclosed herein comprises from, e.g., 1 to 2 silicones, 1 to 3 silicones, 1 to 4 silicones, 1 to 5 silicones, 2 to 3 silicones, 2 to 4 silicones, 2 to 5 silicones, 3 to 4 silicones, 3 to 5 silicones or 4 to 5 silicones.

In another embodiment, a silicone present in a composition disclosed herein comprises a silicone amine, an anionic silicone or any combination thereof. In an aspect of this embodiment, a composition disclosed herein comprises only one silicone amine or a plurality of silicone amines. In another aspect of this embodiment, a composition disclosed herein comprises only one anionic silicone or a plurality of anionic silicones. In yet another aspect of this embodiment, a composition disclosed herein comprises only one anionic silicone and only one anionic silicone. In yet another aspect of this embodiment, a composition disclosed herein comprises a plurality of anionic silicones and a plurality of anionic silicones.

In aspects of this embodiment, a composition disclosed herein comprises, e.g., one or more silicone amines, two or more silicone amines, three or more silicone amines, four or more silicone amines or five or more silicone amines. In other aspects of this embodiment, a composition disclosed herein comprises, e.g., only one silicone amine, at most two silicone amines, at most three silicone amines, at most four silicone amines, or at most five silicone amines. In yet other aspects of this embodiment, a composition disclosed herein comprises from, e.g., 1 to 2 silicone amines, 1 to 3 silicone amines, 1 to 4 silicone amines, 1 to 5 silicone amines, 2 to 3 silicone amines, 2 to 4 silicone amines, 2 to 5 silicone amines, 3 to 4 silicone amines, 3 to 5 silicone amines or 4 to 5 silicone amines.

In aspects of this embodiment, a composition disclosed herein comprises, e.g., one or more anionic silicones, two or more anionic silicones, three or more anionic silicones, four or more anionic silicones or five or more anionic silicones. In other aspects of this embodiment, a composition disclosed herein comprises, e.g., only one anionic silicone, at most two anionic silicones, at most three anionic silicones, at most four anionic silicones, or at most five anionic silicones. In yet other aspects of this embodiment, a composition disclosed herein comprises from, e.g., 1 to 2 anionic silicones, 1 to 3 anionic silicones, 1 to 4 anionic silicones, 1 to 5 anionic silicones, 2 to 3 anionic silicones, 2 to 4 anionic silicones, 2 to 5 anionic silicones, 3 to 4 anionic silicones, 3 to 5 anionic silicones or 4 to 5 anionic silicones.

In aspects of this embodiment, a composition disclosed herein comprises, e.g., one or more silicone amines, two or more silicone amines, three or more silicone amines, four or more silicone amines or five or more silicone amines and, e.g., one or more anionic silicones, two or more anionic silicones, three or more anionic silicones, four or more anionic silicones or five or more anionic silicones. In other aspects of this embodiment, a composition disclosed herein comprises, e.g., only one silicone amine, at most two silicone amines, at most three silicone amines, at most four silicone amines, or at most five silicone amines and e.g., only one anionic silicone, at most two anionic silicones, at most three anionic silicones, at most four anionic silicones, or at most five anionic silicones. In yet other aspects of this embodiment, a composition disclosed herein comprises from, e.g., 1 to 2 silicone amines, 1 to 3 silicone amines, 1 to 4 silicone amines, 1 to 5 silicone amines, 2 to 3 silicone amines, 2 to 4 silicone amines, 2 to 5 silicone amines, 3 to 4 silicone amines, 3 to 5 silicone amines or 4 to 5 silicone amines and e.g., 1 to 2 anionic silicones, 1 to 3 anionic silicones, 1 to 4 anionic silicones, 1 to 5 anionic silicones, 2 to 3 anionic silicones, 2 to 4 anionic silicones, 2 to 5 anionic silicones, 3 to 4 anionic silicones, 3 to 5 anionic silicones or 4 to 5 anionic silicones.

In another embodiment, a composition disclosed herein comprises an amount of silicone that provides a desired beneficial effect to an anti-aging composition and/or a hair smoothing composition disclosed herein. In aspects of this embodiment, a composition disclosed herein comprises a silicone in an amount of, e.g., 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, 10%, 15%, 20%, 25%, 30%, 35%, or 40% by weight of the composition. In other aspects of this embodiment, a composition disclosed herein comprises a silicone in an amount of, e.g., at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, or at least 40% by weight of the composition. In yet other aspects of this embodiment, a composition disclosed herein comprises a silicone in an amount of, e.g., at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, or at most 40% by weight of the composition.

In still other aspects of this embodiment, a composition disclosed herein comprises a silicone in an amount of from, e.g., 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.

In another embodiment, a composition disclosed herein comprises a silicone amine and an anionic silicone in a ratio of from, e.g., about 1:20 to about 20:1, about 1:15 to about 15:1, about 1:10 to about 10:1, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, or about 1:1.

A composition disclosed herein may comprise a rheology agent. A rheology agent modifies the flow properties and characteristics of a compositions disclosed herein. For example, a rheology agent may impart viscosity control, thixotropic flow and thermostable viscosity, increase stability, suspension and syneresis control, and improve the feel, slip and silkiness of a composition. Non-limiting examples of a rheology agent includes a polyurethane-based polymer like bis-lauryl cocaminopropylamine/HDI/PEG-100 copolymer (and) butylene glycol (RHEOLUXE® 812) or bis-C₁₆₋₂₀ isoalkoxy TMHDI/PEG-90 copolymer (RHEOLUXE® 880), a polyether polyol-based polymer like PEG/PPG-450/50 trimethylolpropane dodecyl ether (RHEOLUXE® 8015), a castor oil derivative like trihydroxystearin (THIXCIN® R), a silica (also known as silicon dioxide, a compound that is an oxide of silicon with the chemical formula SiO₂), a hydrophilic clay like a hectorite clay (BENTONE® product line) or a silicate clay like a (LAPONITE® product line), and a steareth-100/PEG-136/HDI copolymer (RHEOLATE® FX 1100). A silicate clay includes sodium lithium magnesium silicate (LAPONITE® XLG), sodium magnesium fluorosilicate (LAPONITE® XL), LAPONITE® XLS and LAPONITE® XL21.

In one embodiment, a single rheology agent is present in a composition disclosed herein. In another embodiment, a plurality of rheology agents is present in a composition disclosed herein. In aspects of this embodiment, a composition disclosed herein comprises, e.g., one or more rheology agents, two or more rheology agents, three or more rheology agents, four or more rheology agents or five or more rheology agents. In other aspects of this embodiment, a composition disclosed herein comprises, e.g., only one rheology agent, at most two rheology agents, at most three rheology agents, at most four rheology agents, or at most five rheology agents. In yet other aspects of this embodiment, a composition disclosed herein comprises from, e.g., 1 to 2 rheology agents, 1 to 3 rheology agents, 1 to 4 rheology agents, 1 to 5 rheology agents, 2 to 3 rheology agents, 2 to 4 rheology agents, 2 to 5 rheology agents, 3 to 4 rheology agents, 3 to 5 rheology agents or 4 to 5 rheology agents.

In another embodiment, a composition disclosed herein comprises an amount of a rheology agent that provides a desired beneficial effect to an anti-aging composition and/or a hair smoothing composition disclosed herein. In aspects of this embodiment, a composition disclosed herein comprises a rheology agent in an amount of, e.g., about 0.001%, about 0.0025%, about 0.005%, about 0.0075%, 0.01%, about 0.025%, about 0.05%, about 0.075%, about 0.1%, about 0.2%, about 0.25%, about 0.3%, about 0.4%, about 0.5%, about 0.6%, about 0.7%, about 0.75%, about 0.8%, about 0.9%, about 1%, about 2%, about 3%, about 4%, about 5%, about 6%, about 7%, about 8%, about 9%, or about 10% by weight of the composition. In other aspects of this embodiment, a composition disclosed herein comprises a rheology agent in an amount of, e.g., at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a composition disclosed herein comprises a rheology agent in an amount of, e.g., at most 0.001%, at most 0.0025%, at most 0.005%, at most 0.0075%, at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a composition disclosed herein comprises a rheology agent in an amount of from, e.g., about 0.001%, about 0.0025%, about 0.005%, about 0.0075%, about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

In another embodiment, a composition disclosed herein comprises an amount of a silica that provides a desired beneficial effect to an anti-aging composition and/or a hair smoothing composition disclosed herein. In aspects of this embodiment, a composition disclosed herein comprises a silica in an amount of, e.g., about 0.001%, about 0.0025%, about 0.005%, about 0.0075%, about 0.01%, about 0.025%, about 0.05%, about 0.075%, about 0.1%, about 0.2%, about 0.25%, about 0.3%, about 0.4%, about 0.5%, about 0.6%, about 0.7%, about 0.75%, about 0.8%, about 0.9%, about 1%, about 2%, about 3%, about 4%, about 5%, about 6%, about 7%, about 8%, about 9%, or about 10% by weight of the composition. In other aspects of this embodiment, a composition disclosed herein comprises a silica in an amount of, e.g., at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a composition disclosed herein comprises a silica in an amount of, e.g., at most 0.001%, at most 0.0025%, at most 0.005%, at most 0.0075%, at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a composition disclosed herein comprises a silica in an amount of from, e.g., about 0.001% to about 0.005%, 0.001% to about 0.0075%, about 0.001% to about 0.01%, about 0.001% to about 0.05%, about 0.001% to about 0.075%, about 0.001% to about 0.1%, about 0.001% to about 0.25%, about 0.001% to about 0.5%, about 0.001% to about 0.75%, about 0.005% to about 0.0075%, about 0.005% to about 0.01%, about 0.005% to about 0.05%, about 0.005% to about 0.075%, about 0.005% to about 0.1%, about 0.005% to about 0.25%, about 0.005% to about 0.5%, about 0.005% to about 0.75%, about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

A composition disclosed herein possesses a certain viscosity depending on the final formulated use. For example, a spray will have a low viscosity while a gel will have a higher viscosity. In aspects of this embodiment, a composition disclosed here has a viscosity of, e.g., about 100 mPa·s, about 250 mPa·s, about 500 mPa·s, about 750 mPa·s, about 1,000 mPa·s, about 1,250 mPa·s, about 1,500 mPa·s, about 1,750 mPa·s, about 2,000 mPa·s, about 2,250 mPa·s, about 2,500 mPa·s, about 2,750 mPa·s, about 3,000 mPa·s, about 3,250 mPa·s, about 3,500 mPa·s, about 3,750 mPa·s, about 4,000 mPa·s, about 4,250 mPa·s, about 4,500 mPa·s, about 4,750 mPa·s, about 5,000 mPa·s, about 5,250 mPa·s, about 5,500 mPa·s, about 5,750 mPa·s, about 6,000 mPa·s, about 6,250 mPa·s, about 6,500 mPa·s, about 6,750 mPa·s, about 7,000 mPa·s, about 7,250 mPa·s, about 7,500 mPa·s, about 7,750 mPa·s, about 8,000 mPa·s, about 8,250 mPa·s, about 8,500 mPa·s, about 8,750 mPa·s, about 9,000 mPa·s, about 9,250 mPa·s, about 9,500 mPa·s, about 9,750 mPa·s, about 10,000 mPa·s, about 10,250 mPa·s, about 10,500 mPa·s, about 10,750 mPa·s, about 11,000 mPa·s, about 11,250 mPa·s, about 11,500 mPa·s, about 11,750 mPa·s, about 12,000 mPa·s, about 12,250 mPa·s, about 12,500 mPa·s, about 12,750 mPa·s, about 13,000 mPa·s, about 13,250 mPa·s, about 13,500 mPa·s, about 13,750 mPa·s, about 14,000 mPa·s, about 14,250 mPa·s, about 14,500 mPa·s, about 14,750 mPa·s, about 15,000 mPa·s, about 15,500 mPa·s, about 16,000 mPa·s, about 16,500 mPa·s, about 17,000 mPa·s, about 17,500 mPa·s, about 18,000 mPa·s, about 18,500 mPa·s, about 19,000 mPa·s, about 19,500 mPa·s, or about 20,000 mPa·s. In other aspects of this embodiment, a composition disclosed here has a viscosity of, e.g., at least 100 mPa·s, at least 500 mPa·s, at least 1,000 mPa·s, at least 2,000 mPa·s, at least 3,000 mPa·s, at least 4,000 mPa·s, at least 5,000 mPa·s, at least 6,000 mPa·s, at least 7,000 mPa·s, at least 8,000 mPa·s, at least 9,000 mPa·s, at least 10,000 mPa·s, at least 11,000 mPa·s, at least 12,000 mPa·s, at least 13,000 mPa·s, at least 14,000 mPa·s, at least 15,000 mPa·s, at least 16,000 mPa·s, at least 17,000 mPa·s, at least 18,000 mPa·s, at least 19,000 mPa·s, or at least 20,000 mPa·s. In yet other aspects of this embodiment, a composition disclosed here has a viscosity of, e.g., at most 100 mPa·s, at most 500 mPa·s, at most 1,000 mPa·s, at most 2,000 mPa·s, at most 3,000 mPa·s, at most 4,000 mPa·s, at most 5,000 mPa·s, at most 6,000 mPa·s, at most 7,000 mPa·s, at most 8,000 mPa·s, at most 9,000 mPa·s, at most 10,000 mPa·s, at most 11,000 mPa·s, at most 12,000 mPa·s, at most 13,000 mPa·s, at most 14,000 mPa·s, at most 15,000 mPa·s, at most 16,000 mPa·s, at most 17,000 mPa·s, at most 18,000 mPa·s, at most 19,000 mPa·s, or at most 20,000 mPa·s.

In still other aspects of this embodiment, a composition disclosed here has a viscosity of, e.g., about 100 mPa·s to about 500 mPa·s, about 100 mPa·s to about 1,000 mPa·s, about 100 mPa·s to about 2,000 mPa·s, about 100 mPa·s to about 3,000 mPa·s, about 100 mPa·s to about 4,000 mPa·s, about 100 mPa·s to about 5,000 mPa·s, about 100 mPa·s to about 6,000 mPa·s, about 100 mPa·s to about 7,000 mPa·s, about 100 mPa·s to about 8,000 mPa·s, about 100 mPa·s to about 9,000 mPa·s, about 100 mPa·s to about 10,000 mPa·s, about 100 mPa·s to about 11,000 mPa·s, about 100 mPa·s to about 12,000 mPa·s, about 100 mPa·s to about 13,000 mPa·s, about 100 mPa·s to about 14,000 mPa·s, about 100 mPa·s to about 15,000 mPa·s, about 100 mPa·s to about 16,000 mPa·s, about 100 mPa·s to about 17,000 mPa·s, about 100 mPa·s to about 18,000 mPa·s, about 100 mPa·s to about 19,000 mPa·s, or about 100 mPa·s to about 20,000 mPa·s, about 1,000 mPa·s to about 2,000 mPa·s, about 1,000 mPa·s to about 3,000 mPa·s, about 1,000 mPa·s to about 4,000 mPa·s, about 1,000 mPa·s to about 5,000 mPa·s, about 1,000 mPa·s to about 6,000 mPa·s, about 1,000 mPa·s to about 7,000 mPa·s, about 1,000 mPa·s to about 8,000 mPa·s, about 1,000 mPa·s to about 9,000 mPa·s, about 1,000 mPa·s to about 10,000 mPa·s, about 1,000 mPa·s to about 11,000 mPa·s, about 1,000 mPa·s to about 12,000 mPa·s, about 1,000 mPa·s to about 13,000 mPa·s, about 1,000 mPa·s to about 14,000 mPa·s, about 1,000 mPa·s to about 15,000 mPa·s, about 1,000 mPa·s to about 16,000 mPa·s, about 1,000 mPa·s to about 17,000 mPa·s, about 1,000 mPa·s to about 18,000 mPa·s, about 1,000 mPa·s to about 19,000 mPa·s, or about 1,000 mPa·s to about 20,000 mPa·s, about 2,000 mPa·s to about 3,000 mPa·s, about 2,000 mPa·s to about 4,000 mPa·s, about 2,000 mPa·s to about 5,000 mPa·s, about 2,000 mPa·s to about 6,000 mPa·s, about 2,000 mPa·s to about 7,000 mPa·s, about 2,000 mPa·s to about 8,000 mPa·s, about 2,000 mPa·s to about 9,000 mPa·s, about 2,000 mPa·s to about 10,000 mPa·s, about 2,000 mPa·s to about 11,000 mPa·s, about 2,000 mPa·s to about 12,000 mPa·s, about 2,000 mPa·s to about 13,000 mPa·s, about 2,000 mPa·s to about 14,000 mPa·s, about 2,000 mPa·s to about 15,000 mPa·s, about 2,000 mPa·s to about 16,000 mPa·s, about 2,000 mPa·s to about 17,000 mPa·s, about 2,000 mPa·s to about 18,000 mPa·s, about 2,000 mPa·s to about 19,000 mPa·s, or about 2,000 mPa·s to about 20,000 mPa·s, about 2,500 mPa·s to about 5,000 mPa·s, about 2,500 mPa·s to about 7,500 mPa·s, about 2,500 mPa·s to about 10,000 mPa·s, about 2,500 mPa·s to about 12,500 mPa·s, about 2,500 mPa·s to about 15,000 mPa·s, about 2,500 mPa·s to about 17,500 mPa·s, about 2,500 mPa·s to about 20,000 mPa·s, about 5,000 mPa·s to about 7,500 mPa·s, about 5,000 mPa·s to about 10,000 mPa·s, about 5,000 mPa·s to about 12,500 mPa·s, about 5,000 mPa·s to about 15,000 mPa·s, about 5,000 mPa·s to about 17,500 mPa·s, about 5,000 mPa·s to about 20,000 mPa·s, about 7,500 mPa·s to about 10,000 mPa·s, about 7,500 mPa·s to about 12,500 mPa·s, about 7,500 mPa·s to about 15,000 mPa·s, about 7,500 mPa·s to about 17,500 mPa·s, about 7,500 mPa·s to about 20,000 mPa·s, about 10,000 mPa·s to about 12,500 mPa·s, about 10,000 mPa·s to about 15,000 mPa·s, about 10,000 mPa·s to about 17,500 mPa·s, about 10,000 mPa·s to about 20,000 mPa·s, about 12,500 mPa·s to about 15,000 mPa·s, about 12,500 mPa·s to about 17,500 mPa·s, about 12,500 mPa·s to about 20,000 mPa·s, about 15,000 mPa·s to about 17,500 mPa·s, about 15,000 mPa·s to about 20,000 mPa·s, or about 17,500 mPa·s to about 20,000 mPa·s.

A composition disclosed herein may comprise a carrier. A carrier, also known as a vehicle, can be any material typically known in the skin care, cosmetic and medical arts that is used as a base to formulate a composition disclosed herein. A carrier may be an aqueous carrier, a semi-solid carrier or a solid carrier. A carrier can also provide a skin care benefit as disclosed herein. A carrier includes, without limitation, water, a vegetable oil, a mineral oil, an ester oil, an ether, an alcohol, a fatty alcohol, an isoparaffin, a hydrocarbon oil, a polyol, and a wax. Non-limiting examples of an ester oil include octal palmitate, isopropyl myristate and isopropyl palmitate. Non-limiting examples of an ether includes dicapryl ether and dimethyl isosorbide. Non-limiting examples of an alcohol includes ethanol and isopropanol. Non-limiting examples of a fatty alcohol include cetyl alcohol, cetearyl alcohol, stearyl alcohol and behenyl alcohol. Non-limiting examples of an isoparaffin include isooctane, isododecane (IDD) and isohexadecane. Non-limiting examples of a hydrocarbon oil include mineral oil, petrolatum, isoeicosane and a polyolefin, including (hydrogenated) polyisobutene. Non-limiting examples of a polyol include propylene glycol, glycerin, butylene glycol, pentylene glycol, hexylene glycol, caprylyl glycol. Non-limiting examples of a wax include beeswax, carnauba, ozokerite, microcrystalline wax, polyethylene wax, and a botanical wax.

In one embodiment, a single carrier is present in a composition disclosed herein. In another embodiment, a plurality of carriers is present in a composition disclosed herein. In aspects of this embodiment, a composition disclosed herein comprises, e.g., one or more carriers, two or more carriers, three or more carriers, four or more carriers or five or more carriers. In other aspects of this embodiment, a composition disclosed herein comprises, e.g., only one carrier, at most two carriers, at most three carriers, at most four carriers, or at most five carriers. In yet other aspects of this embodiment, a composition disclosed herein comprises from, e.g., 1 to 2 carriers, 1 to 3 carriers, 1 to 4 carriers, 1 to 5 carriers, 2 to 3 carriers, 2 to 4 carriers, 2 to 5 carriers, 3 to 4 carriers, 3 to 5 carriers or 4 to 5 carriers.

In another embodiment, a composition disclosed herein comprises an amount of carrier that provides a desired formulative or beneficial effect to an anti-aging composition and/or a hair smoothing composition disclosed herein. In aspects of this embodiment, a composition disclosed herein comprises a carrier in an amount of, e.g., at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% by weight of the composition. In other aspects of this embodiment, a composition disclosed herein comprises a carrier in an amount of, e.g., at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98% or at most 99% by weight of the composition. In yet other aspects of this embodiment, a composition disclosed herein comprises a carrier in an amount of from, e.g., about 25% to about 50%, about 25% to about 75%, about 25% to about 90%, about 25% to about 95%, about 25% to about 96%, about 25% to about 97%, about 25% to about 98%, about 25% to about 99%, about 50% to about 75%, about 50% to about 90%, about 50% to about 95%, about 50% to about 96%, about 50% to about 97%, about 50% to about 98%, about 50% to about 99%, about 75% to about 80%, about 75% to about 85%, about 75% to about 90%, about 75% to about 95%, about 75% to about 96%, about 75% to about 97%, about 75% to about 98%, about 75% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, or about 95% to about 99%, by weight of the composition.

A composition disclosed herein may further optionally include additional ingredients. An additional ingredient is one known to be useful in finishing an anti-aging composition and/or a hair smoothing composition disclosed herein. An additional ingredient includes, without limitation, a surfactant, a buffer, a humectant, a preservative, an emulsifier, a fragrance, a chelating agent, an astringent, a cosmetic agent, a shine control agent, a colorant agent, a soothing agent, and a calming agent. An additional ingredient disclosed herein is known in the art.

Surfactants are compounds that lower the surface tension of a liquid, allowing easier spreading, and lowering of the interfacial tension between two liquids, or between a liquid and a solid. Either a single surfactant may be mixed with the buffered solution disclosed herein, or a plurality of surfactants may be mixed with the buffered solution disclosed herein. Useful surfactants, include, without limitation, ionic surfactants, zwitterionic (amphoteric) surfactants, non-ionic surfactants, or any combination therein. Surfactants are useful, e.g., when formulating a shampoo, a conditioner, or a body wash. The surfactant used in a composition disclosed herein can be varied as appropriate by one skilled in the art and generally depends, in part, on the particular buffer being used, the protein being eluted, and the conductivity values being employed.

Ionic surfactants include anionic surfactants. Anionic surfactants include ones based on permanent functional groups attached to the head, such as, e.g., sulfate, sulfonate, phosphate carboxylates) or pH dependent anionic surfactants. Anionic surfactants include, without limitation, alkyl sulfates like ammonium lauryl sulfate and sodium lauryl sulfate (SDS); alkyl ether sulfates like sodium laureth sulfate and sodium myreth sulfate; docusates like dioctyl sodium sulfosuccinate; sulfonate fluorosurfactants like perfluorooctanesulfonate (PFOS) and perfluorobutanesulfonate; alkyldiphenyloxide Disulfonates like DOWFAX™ 2A1 (Disodium Lauryl Phenyl Ether Disulfonate), DOWFAX™ 3B2 (Disodium Decyl Phenyl Ether Disulfonate), DOWFAX™ C10L (Disodium Decyl Phenyl Ether Disulfonate), DOWFAX™ 2EP, and DOWFAX™ 8390 (Disodium Cetyl Phenyl Ether Disulfonate); potassium phosphate polyether esters like TRITON™ H-55 and TRITON™ H-66; alkyl benzene sulfonates; alkyl aryl ether phosphates; alkyl ether phosphates; alkyl carboxylates like fatty acid salts and sodium stearate; sodium lauroyl sarcosinate; carboxylate fluorosurfactants like perfluorononanoate and perfluorooctanoate; and Sodium Hexyldiphenyl Ether Sulfonate (DOWFAX™ C6L).

Ionic surfactants also include cationic surfactants. Cationic surfactants include ones based on permanent or pH dependent cationic surfactants, such as, e.g., primary, secondary or tertiary amines. Cationic surfactants include, without limitation, alkyltrimethylammonium salts like cetyl trimethylammonium bromide (CTAB) and cetyl trimethylammonium chloride (CTAC); cetylpyridinium chloride (CPC); polyethoxylated tallow amine (POEA); benzalkonium chloride (BAC); benzethonium chloride (BZT); 5-Bromo-5-nitro-1,3-dioxane; dimethyldioctadecylammonium chloride; and dioctadecyldimethylammonium bromide (DODAB), as well as pH-dependent primary, secondary or tertiary amines like surfactants where the primary amines become positively charged at pH greater than 10, or the secondary amines become charged at pH less than 4, like octenidine dihydrochloride. Other useful anionic surfactants include bio-based anionic surfactants, including, without limitation, STEPONOL® AM 30-KE, an ammonium lauryl sulfate, and STEPONOL® EHS, a sodium 2-ethyl hexyl sulfate. Such bio-based surfactants are not synthetic molecules, but instead are anionic biosurfactants derived from organic matter such as plants.

Zwitterionic surfactants are based on primary, secondary or tertiary amines or quaternary ammonium cation with a sulfonate, a carboxylate, or a phosphate. Zwitterionic surfactants include, without limitation, 3-[(3-Cholamidopropyl)dimethylammonio]-1-propanesulfonate (CHAPS); sultaines like cocamidopropyl hydroxysultaine; betaines like cocamidopropyl betaine; or lecithins.

Non-ionic surfactants are less denaturing and as such are useful to solubilize membrane proteins and lipids while retaining protein-protein interactions. Nonionic surfactant include polyether nonionic surfactants, polyhydroxyl nonionic surfactants and biosurfactants. Nonionic surfactant include alcohol ethoxylates, alkylphenol ethoxylates, phenol ethoxylates, amide ethoxylates, glyceride ethoxylates, fatty acid ethoxylates, and fatty amine ethoxylates. Other useful non-ionic surfactants include bio-based non-ionic surfactants, including, without limitation, STEPOSOL® MET-10U, a metathesis-derived, nonionic surfactant that is an unsaturated, short chain amide. Such bio-based surfactants are not synthetic molecules, but instead are non-ionic biosurfactants derived from organic matter such as plants.

Non-limiting examples of surfactants include polyoxyethylene glycol sorbitan alkyl esters (or ethoxylated sorbital esters) like polysorbate 20 sorbitan monooleate (TWEEN® 20), polysorbate 40 sorbitan monooleate (TWEEN® 40), polysorbate 60 sorbitan monooleate (TWEEN® 60), polysorbate 61 sorbitan monooleate (TWEEN® 61), polysorbate 65 sorbitan monooleate (TWEEN® 65), polysorbate 80 sorbitan monooleate (TWEEN® 80), polysorbate 81 sorbitan monooleate (TWEEN® 81) and polysorbate 85 sorbitan monooleate (TWEEN® 85); sorbital esters like sorbitan monooleate, sorbitan monolaurate, sorbitan monopalmitate, sorbitan monostearate and sorbitan tristearate; polyglycerol esters like glycerol monooleate, glycerol monolaurate, glycerol monopalmitate, glycerol monostearate, glycerol trioleate, glycerol ricinoleate, glycerol tristearate, mono diglycerides and glycerol triacetate; ethoxylated polyglycerol esters; alkyl glucosides like arachidyl glucoside, C₁₂₋₂₀ alkyl glucoside, caprylyl/capryl glucoside, cetearyl glucoside, coco-glucoside, ethyl glucoside and lauryl glucoside. decyl glucoside; ethoxylated alkyl glucosides; sucrose esters like sucrose monooleate, sucrose monolaurate, sucrose monopalmitate, sucrose monostearate, sucrose trioleate, sucrose ricinoleate, sucrose tristearate, sucrose diglycerides and sucrose triacetate; ethoxylated sucrose ester; amine oxides; ethoxylated alcohols; ethoxylated aliphatic alcohols; alkylamines; ethoxylated alkylamines; ethoxylated alkyl phenols like ethoxylated nonyl phenol and ethoxylated octyl phenol; alkyl polysaccharides; ethoxylated alkyl polysaccharides; ethoxylated fatty acids like ethoxylated castor oil; ethoxylated fatty alcohols like ethoxylated ceto-oleyl alcohol, ethoxylated ceto-stearyl alcohol, ethoxylated decyl alcohol, ethoxylated dodecyl alcohol and ethoxylated tridecyl alcohol; ethoxylated fatty amines; poloxamers (polyethylene-polypropylene copolymers), like Poloxamer 124 (PLURONIC® L44), Poloxamer 181 (PLURONIC® L61), Poloxamer 182 (PLURONIC® L62), Poloxamer 184 (PLURONIC® L64), Poloxamer 188 (PLURONIC® F68), Poloxamer 237 (PLURONIC® F87), Poloxamer 338 (PLURONIC® L108), and Poloxamer 407 (PLURONIC® F127); linear secondary alcohol ethoxylates like TERGITOL™ 15-S-5, TERGITOL™ 15-S-7, TERGITOL™ 15-S-9, TERGITOL™ 15-S-12, TERGITOL™ 15-S-15, TERGITOL™ 15-S-20, TERGITOL™ 15-S-30 and TERGITOL™ 15-S-40; alkyl phenol polyglycol ethers; polyethylene glycol alkyl aryl ethers; polyoxyethylene glycol alkyl ethers, like octaethylene glycol monododecyl ether, pentaethylene glycol monododecyl ether, BRIJ® 30, and BRIJ® 35; 2-dodecoxyethanol (LUBROL®-PX); polyoxyethylene glycol octylphenol ethers like polyoxyethylene (4-5) p-t-octyl phenol (TRITON® X-45) and polyoxyethylene octyl phenyl ether (TRITON® X-100); polyoxyethylene glycol alkylphenol ethers like Nonoxynol-9; phenoxypolyethoxylethanols like nonylphenoxypolyethoxylethanol and octylphenoxypolyethoxylethanol (IGEPAL® CA-630 or NONIDET™ P-40); glucoside alkyl ethers like octyl glucopyranoside; maltoside alkyl ethers like dodecyl maltopyranoside; thioglucoside alkyl ethers like heptyl thioglucopyranoside; digitonins; glycerol alkyl esters like glyceryl laurate; alkyl aryl polyether sulfates; alcohol sulfonates; sorbitan alkyl esters; cocamide ethanolamines like cocamide monoethanolamine and cocamide diethanolamine; sucrose monolaurate; dodecyl dimethylamine oxide, and sodium cholate. Other non-limiting examples of surfactants useful in the methods disclosed herein can be found in, e.g., Winslow, et al., Methods and Compositions for Simultaneously Isolating Hemoglobin from Red Blood Cells and Inactivating Viruses, U.S. 2008/0138790; Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

In one embodiment, a single surfactant is present in a composition disclosed herein. In another embodiment, a plurality of surfactants is present in a composition disclosed herein. In aspects of this embodiment, a composition disclosed herein comprises, e.g., one or more surfactants, two or more surfactants, three or more surfactants, four or more surfactants or five or more surfactants. In other aspects of this embodiment, a composition disclosed herein comprises, e.g., only one surfactant, at most two surfactants, at most three surfactants, at most four surfactants, or at most five surfactants. In yet other aspects of this embodiment, a composition disclosed herein comprises from, e.g., 1 to 2 surfactants, 1 to 3 surfactants, 1 to 4 surfactants, 1 to 5 surfactants, 2 to 3 surfactants, 2 to 4 surfactants, 2 to 5 surfactants, 3 to 4 surfactants, 3 to 5 surfactants or 4 to 5 surfactants.

Any amount of a surfactant disclosed herein may be used, with the proviso that the amount is useful to practice the methods disclosed herein. In aspects of this embodiment, a composition disclosed herein comprises a surfactant in an amount of, e.g., about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0% or about 10.0% by weight of the composition. In other aspects of this embodiment, a composition disclosed herein comprises a surfactant in an amount of, e.g., at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, or at least 10.0% by weight of the composition. In yet other aspects of this embodiment, a composition disclosed herein comprises a surfactant in an amount of, e.g., at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0% or at most 10.0% by weight of the composition.

In still other aspects of this embodiment, a composition disclosed herein comprises a surfactant in an amount of, e.g., about 0.1% to about 0.5%, about 0.1% to about 0.75%, about 0.1% to about 1.0%, about 0.1% to about 1.5%, about 0.1% to about 2.0%, about 0.1% to about 2.5%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0% or about 5.0% to about 15.0% by weight of the composition.

In another embodiment, a composition disclosed herein does not comprise a surfactant.

In another embodiment, a composition disclosed herein does not comprise a buffer or buffered solution. A buffer or buffered solution can be varied as appropriate by one skilled in the art and generally depends, in part, on the pH value desired for the mobile phase, the protein being eluted, and the conductivity values being employed. Therefore, aspects of this embodiment may optionally include, e.g., 2-(N-morpholino) ethanesulfonic acid (MES), N-(2-acetamido)iminodiacetic acid (ADA), dimethylarsinic acid (Cacodylate), piperazine-N,N′-bis(2-ethanesulfonic acid) (PIPES), N-(2-Acetamido)-2-aminoethanesulfonic acid (ACES), cholamine chloride, N,N′-bis(2-hydroxyethyl)-2-aminoethanesulfonic acid (BES), 3-(N-morpholino) propanesulfonic acid (MOPS), 2-{[tris(hydroxymethyl)methyl]amino}ethanesulfonic acid (TES), N-(2-hydroxyethyl) piperazine-N′-(2-ethanesulfonic acid) (HEPES), piperazine-N,N′-bis(2-hydroxypropanesulfonic acid) (POPSO), N-tris(hydroxymethyl) methylglycine (Tricine), tris(hydroxymethyl)methylamine (Tris), acetamidoglycine, N,N-bis(2-hydroxyethyl)glycine (Bicine), N-tris(hydroxymethyl)methyl-3-aminopropanesulfonic acid (TAPS), 3-[(1,1-dimethyl-2-hydroxyethyl)amino]-2-hydroxypropanesulfonic acid (AMPSO), 3-(cyclohexylamino)-2-hydroxy-1-propanesulfonic acid (CAPSO), and 3-(cyclohexylamino)-1-propanesulfonic acid (CAPS); acetate buffers, such as, e.g., magnesium acetate, potassium acetate, and Tris acetate; borate buffers; citrate buffers; phosphate buffers, such as, e.g., potassium phosphate buffers and sodium phosphate buffers; saline buffers, such as, e.g., phosphate-buffered saline (PBS), HEPES-buffered saline (HBS), and Tris-buffered saline (TBS), saline sodium citrate (SSC); universal buffers, such as, e.g., buffers comprising citric acid and potassium phosphate, Britton-Robinson buffer, Carmody buffer and the like, or any combination thereof. Non-limiting examples of how to make and use specific buffers are described in, e.g., MOLECULAR CLONING, A LABORATORY MANUAL (Joseph Sambrook & David W. Russell eds., Cold Spring Harbor Laboratory Press, 3rd ed. 2001) and CURRENT PROTOCOLS IN MOLECULAR BIOLOGY (Frederick M. Ausubel et al., eds. John Wiley & Sons, 2004).

It is understood that acids or bases can be used to adjust the pH of a buffered solution disclosed herein as needed. In an aspect of this embodiment, an effective pH level of a buffered solution disclosed herein is, e.g., at least about pH 5.0, at least about pH 5.5, at least about pH 6.0, at least about pH 6.5, at least about pH 7.0 or at about pH 7.5. In another aspect of this embodiment, an effective pH level of a buffered solution disclosed herein is, e.g., at most about pH 5.0, at most about pH 5.5, at most about pH 6.0, at most about pH 6.5, at most about pH 7.0 or at most about pH 7.5. In yet another aspect of this embodiment, an effective pH level of a buffered solution disclosed herein is, e.g., about pH 5.0 to about pH 8.0, an effective pH level is about pH 5.0 to about pH 7.0, an effective pH level is about pH 5.0 to about pH 6.0, is about pH 5.5 to about pH 8.0, an effective pH level is about pH 5.5 to about pH 7.0, an effective pH level is about pH 5.5 to about pH 5.0, is about pH 5.5 to about pH 7.5, an effective pH level is about pH 5.5 to about pH 6.5.

A buffered solution disclosed herein may be of any concentration, with the proviso that the concentration is useful to practice the methods disclosed herein. The buffer concentration used can be varied as appropriate by one skilled in the art and generally depends, in part, on the buffering capacity of a particular buffer being used and the other components of a composition disclosed herein. In aspects of this embodiment, the concentration of a buffered solution is, e.g., at least 0.1 mM, at least 0.2 mM, at least 0.3 mM, at least 0.4 mM, at least 0.5 mM, at least 0.6 mM, at least 0.7 mM, at least 0.8 mM, or at least 0.9 mM. In other aspects of this embodiment, the buffer concentration of a buffered solution is, e.g., at least 1.0 mM, at least 2.0 mM, at least 3.0 mM, at least 4.0 mM, at least 5.0 mM, at least 6.0 mM, at least 7.0 mM, at least 8.0 mM, or at least 9.0 mM. In yet other aspects of this embodiment, the concentration of a buffered solution is, e.g., at least 10 mM, at least 20 mM, at least 30 mM, at least 40 mM, at least 50 mM, at least 60 mM, at least 70 mM, at least 80 mM, or at least 90 mM. In still other aspects of this embodiment, the concentration of a buffered solution is, e.g., at least 100 mM, at least 200 mM, at least 300 mM, at least 400 mM, at least 500 mM, at least 600 mM, at least 700 mM, at least 800 mM, or at least 900 mM.

In other aspects of this embodiment, the concentration of a buffered solution is, e.g., at most 0.1 mM, at most 0.2 mM, at most 0.3 mM, at most 0.4 mM, at most 0.5 mM, at most 0.6 mM, at most 0.7 mM, at most 0.8 mM, or at most 0.9 mM. In yet other aspects of this embodiment, the concentration of a buffered solution is, e.g., at most 1.0 mM, at most 2.0 mM, at most 3.0 mM, at most 4.0 mM, at most 5.0 mM, at most 6.0 mM, at most 7.0 mM, at most 8.0 mM, or at most 9.0 mM. In still other aspects of this embodiment, the concentration of a buffered solution is, e.g., at most 10 mM, at most 20 mM, at most 30 mM, at most 40 mM, at most 50 mM, at most 60 mM, at most 70 mM, at most 80 mM, or at most 90 mM. In further aspects of this embodiment, the concentration of a buffered solution is, e.g., at most 100 mM, at most 200 mM, at most 300 mM, at most 400 mM, at most 500 mM, at most 600 mM, at most 700 mM, at most 800 mM, or at most 900 mM.

In other aspects of this embodiment, the concentration of a buffered solution is between, e.g., about 0.1 mM to about 900 mM, 0.1 mM to about 500 mM, 0.1 mM to about 100 mM, 0.1 mM to about 90 mM, 0.1 mM to about 50 mM, 1.0 mM to about 900 mM, 1.0 mM to about 500 mM, 1.0 mM to about 100 mM, 1.0 mM to about 90 mM, or 1.0 mM to about 50 mM.

A humectant can elevate the hydration of the skin, in particular the epidermis and the dermis. Non-limiting examples of a humectant include, glycerol, glycerin, lactic acid, lactate, urea, aloe vera, betaine, amino acids, glycosaminoglycans, sorbitol, honey, hyaluronic acid, propanediol, propylene glycol, butylene glycol, a polyol, and hydrogenated starch hydrolysates.

A preservative preserves the stability of a composition disclosed herein. A preservative can also prevent the growth of microbial organisms in a composition disclosed herein. Non-limiting examples of a preservative include methylparaben, phenoxyethanol, capryl glycol, glyceryl caprylate, benzoic acid, sorbic acid, gallic acid, and propylparaben.

An emulsifier creates liquid crystalline networks or liposomal networks. Non-limiting examples of an emulsifier include a lecithin, a hydrogenated lecithin, a lysolecithin, a phosphatidylcholine, and a phospholipid. Commercially available emulsifiers include OLIVEM® 1000 (a cetearyl olivate/sorbitan olivate composition), ARLACEL™ LC (a sorbitan stearate/sorbityl laurate composition), CRYSTALCAST® MM (a beta sitosterol/sucrose stearate/sucrose distearate/cetyl alcohol/stearyl alcohol mixture), and UNIOX CRISTAL (a cetearyl alcohol/polysorbate 60/cetearyl glucoside composition).

An astringent is a substance that causes the contraction of body tissues. Non-limiting examples of an astringent include ethanol, witch hazel, rose water, alum, oatmeal, yarrow, bayberry, cold water, rubbing alcohol, silver nitrate, zinc oxide, zinc sulfate, Burow's solution, tincture of benzoin, and vegetable substances such as tannic and gallic acid.

A cosmetic agent is a substance that aids in the reduction of wrinkles as well as to soften strong, defining lines and improve the overall look and feel of the skin. A cosmetic agent includes, without limitation, α-Hydroxy acids (AHA), β-hydroxy acid (BHA), Hyaluronic Acid (HA), fatty acids, cholesterol and ceramides. An α-hydroxy acid is naturally occurring or synthetic organic acid with a hydroxyl group on the carbon adjacent to the carboxylic acid group. Non-limiting examples of α-hydroxy acids include glycolic acid, lactic acid, citric acid, malic acid, tartaric acid, α-hydroxyoctanoic acid, and α-hydroxydecanoic acid. β-hydroxy acid (BHA) is a naturally occurring or synthetic organic acid that contains a carboxylic acid functional group and hydroxy functional group separated by two carbon atoms. Non-limiting examples of β-hydroxy acid include salicylic acid, β-Hydroxputyric acid, β-Hydroxy β-methylbutyric acid and Carnitine. A fatty acid includes an omega-9 (oleinic) fatty acid, an omega-6 (linolic) fatty acid and an omega-3 (α-linolenic) fatty acid. Non-limiting examples of a fatty acid include Caprylic acid, Capric acid, Caprynic acid, Lauric Acid, Myristic Acid, Palmitic Acid, Palmitoleic acid, Margaric acid, Stearic acid, Oleic Acid, Petroselinic acid, Punicic acid, Ricinolenic acid, linoleic acid, α-Linolenic acid, Linolenic acid, γ-linolenic acid, cis-linolenic acid, Arachidic acid, Arachidonic acid, Eicosenic acid, Behenic acid, Erucic acid, and Lignoceric acid. A ceramide is a naturally occurring, long chains of lipid comprising a sphingoid base and a fatty acid, suhc as, e.g., a N-acylated sphingolipid and a N-acylated phytosphingosine. Non-limiting examples of a ceramide include Ceramide 1, Ceramide 1A, Ceramide 2, Ceramide 3, Ceramide 4, Ceramide 5, Ceramide 6 II, Ceramide AP, Ceramide AS, Ceramide EOP, Ceramide EOS, Ceramide NG, Ceramide NP, Ceramide NS, and Ceramide NS Dilaurate.

A composition disclosed herein may be adjusted to any pH that enables an anti-aging composition and/or a hair smoothing composition disclosed herein to achieve a desired beneficial effect. In one embodiment, the pH of a composition disclosed herein is between, e.g., about 2 to about 5, about 2 to about 5.5, about 2 to about 6, about 2 to about 6.5, about 2 to about 7, about 2 to about 7.5, about 2 to about 8, about 2 to about 8.5, about 2 to about 9, about 2.5 to about 5, about 2.5 to about 5.5, about 2.5 to about 6, about 2.5 to about 6.5, about 2.5 to about 7, about 2.5 to about 7.5, about 2.5 to about 8, about 2.5 to about 8.5, about 2.5 to about 9, about 3 to about 5, about 3 to about 5.5, about 3 to about 6, about 3 to about 6.5, about 3 to about 7, about 3 to about 7.5, about 3 to about 8, about 3 to about 8.5, about 3 to about 9, about 3.5 to about 5, about 3.5 to about 5.5, about 3.5 to about 6, about 3.5 to about 6.5, about 3.5 to about 7, about 3.5 to about 7.5, about 3.5 to about 8, about 3.5 to about 8.5, about 3.5 to about 9, about 4 to about 5, about 4 to about 5.5, about 4 to about 6, about 4 to about 6.5, about 4 to about 7, about 4 to about 7.5, about 4 to about 8, about 4 to about 8.5, about 4 to about 9, about 4.5 to about 5, about 4.5 to about 5.5, about 4.5 to about 6, about 4.5 to about 6.5, about 4.5 to about 7, about 4.5 to about 7.5, about 4.5 to about 8, about 4.5 to about 8.5, about 4.5 to about 9, about 5 to about 5.5, about 5 to about 6, about 5 to about 6.5, about 5 to about 7, about 5 to about 7.5, about 5 to about 8, about 5 to about 8.5, about 5 to about 9, about 5.5 to about 6, about 5.5 to about 6.5, about 5.5 to about 7, about 5.5 to about 7.5, about 5.5 to about 8, about 5.5 to about 8.5, about 5.5 to about 9, about 6 to about 6.5, about 6 to about 7, about 6 to about 7.5, about 6 to about 8, about 6 to about 8.5, about 6 to about 9, about 6.5 to about 7, about 6.5 to about 7.5, about 6.5 to about 8, about 6.5 to about 8.5, about 6.5 to about 9, about 7 to about 7.5, about 7 to about 8, about 7 to about 8.5, about 7 to about 9, about 7.5 to about 8, about 7.5 to about 8.5, about 7.5 to about 9, about 8 to about 8.5, about 8 to about 9, or about 8.5 to about 9.

In another embodiment, the pH of a composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2. In another embodiment, the pH of a composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

A composition disclosed herein is stable. In one embodiment, a composition disclosed herein is stable when it shows, e.g., at most 1% degradation, at most 2% degradation, at most 3% degradation, at most 4% degradation, at most 5% degradation, at most 6% degradation, at most 7% degradation, at most 8% degradation, at most 9% degradation, or at most 10% degradation of hypochlorous acid. In another embodiment, a composition disclosed herein is stable when it shows, e.g., at most 10% degradation, at most 15% degradation, at most 20% degradation, at most 25% degradation, at most 30% degradation, at most 35% degradation, at most 40% degradation, at most 45% degradation, at most 50% degradation, at most 55% degradation, at most 60% degradation, at most 65% degradation, at most 70% degradation, at most 75% degradation, at most 80% degradation, at most 85% degradation, at most 90% degradation, or at most 95% degradation of hypochlorous acid. In another embodiment, a composition disclosed herein is stable when it shows, e.g., about 1% to about 10% degradation, about 1% to about 20% degradation, about 1% to about 30% degradation, about 1% to about 40% degradation, about 1% to about 50% degradation, about 1% to about 60% degradation, about 1% to about 70% degradation, about 1% to about 80% degradation, about 1% to about 90% degradation, about 1% to about 95% degradation, about 5% to about 10% degradation, about 5% to about 20% degradation, about 5% to about 30% degradation, about 5% to about 40% degradation, about 5% to about 50% degradation, about 5% to about 60% degradation, about 5% to about 70% degradation, about 5% to about 80% degradation, about 5% to about 90% degradation, about 5% to about 95% degradation, about 10% to about 20% degradation, about 10% to about 30% degradation, about 10% to about 40% degradation, about 10% to about 50% degradation, about 10% to about 60% degradation, about 10% to about 70% degradation, about 10% to about 80% degradation, about 10% to about 90% degradation, about 10% to about 95% degradation, 20% to about 30% degradation, about 20% to about 40% degradation, about 20% to about 50% degradation, about 20% to about 60% degradation, about 20% to about 70% degradation, about 20% to about 80% degradation, about 20% to about 90% degradation, about 20% to about 95% degradation, about 30% to about 40% degradation, about 30% to about 50% degradation, about 30% to about 60% degradation, about 30% to about 70% degradation, about 30% to about 80% degradation, about 30% to about 90% degradation, about 30% to about 95% degradation, about 40% to about 50% degradation, about 40% to about 60% degradation, about 40% to about 70% degradation, about 40% to about 80% degradation, about 40% to about 90% degradation, about 40% to about 95% degradation, about 50% to about 60% degradation, about 50% to about 70% degradation, about 50% to about 80% degradation, about 50% to about 90% degradation, about 50% to about 95% degradation, about 60% to about 70% degradation, about 60% to about 80% degradation, about 60% to about 90% degradation, about 60% to about 95% degradation, about 70% to about 80% degradation, about 70% to about 90% degradation, about 70% to about 95% degradation, about 80% to about 90% degradation, about 80% to about 95% degradation, or about 90% to about 95% degradation of hypochlorous acid.

In one embodiment, a composition disclosed herein is stable when it shows, e.g., at most 1% degradation, at most 2% degradation, at most 3% degradation, at most 4% degradation, at most 5% degradation, at most 6% degradation, at most 7% degradation, at most 8% degradation, at most 9% degradation, or at most 10% degradation of free available chlorine. In another embodiment, a composition disclosed herein is stable when it shows, e.g., at most 10% degradation, at most 15% degradation, at most 20% degradation, at most 25% degradation, at most 30% degradation, at most 35% degradation, at most 40% degradation, at most 45% degradation, at most 50% degradation, at most 55% degradation, at most 60% degradation, at most 65% degradation, at most 70% degradation, at most 75% degradation, at most 80% degradation, at most 85% degradation, at most 90% degradation, or at most 95% degradation of free available chlorine. In another embodiment, a composition disclosed herein is stable when it shows, e.g., about 1% to about 10% degradation, about 1% to about 20% degradation, about 1% to about 30% degradation, about 1% to about 40% degradation, about 1% to about 50% degradation, about 1% to about 60% degradation, about 1% to about 70% degradation, about 1% to about 80% degradation, about 1% to about 90% degradation, about 1% to about 95% degradation, about 5% to about 10% degradation, about 5% to about 20% degradation, about 5% to about 30% degradation, about 5% to about 40% degradation, about 5% to about 50% degradation, about 5% to about 60% degradation, about 5% to about 70% degradation, about 5% to about 80% degradation, about 5% to about 90% degradation, about 5% to about 95% degradation, about 10% to about 20% degradation, about 10% to about 30% degradation, about 10% to about 40% degradation, about 10% to about 50% degradation, about 10% to about 60% degradation, about 10% to about 70% degradation, about 10% to about 80% degradation, about 10% to about 90% degradation, about 10% to about 95% degradation, 20% to about 30% degradation, about 20% to about 40% degradation, about 20% to about 50% degradation, about 20% to about 60% degradation, about 20% to about 70% degradation, about 20% to about 80% degradation, about 20% to about 90% degradation, about 20% to about 95% degradation, about 30% to about 40% degradation, about 30% to about 50% degradation, about 30% to about 60% degradation, about 30% to about 70% degradation, about 30% to about 80% degradation, about 30% to about 90% degradation, about 30% to about 95% degradation, about 40% to about 50% degradation, about 40% to about 60% degradation, about 40% to about 70% degradation, about 40% to about 80% degradation, about 40% to about 90% degradation, about 40% to about 95% degradation, about 50% to about 60% degradation, about 50% to about 70% degradation, about 50% to about 80% degradation, about 50% to about 90% degradation, about 50% to about 95% degradation, about 60% to about 70% degradation, about 60% to about 80% degradation, about 60% to about 90% degradation, about 60% to about 95% degradation, about 70% to about 80% degradation, about 70% to about 90% degradation, about 70% to about 95% degradation, about 80% to about 90% degradation, about 80% to about 95% degradation, or about 90% to about 95% degradation of free available chlorine.

In aspects of this embodiment, a composition disclosed herein is stable for, e.g., about 1 day, about 2 days, 3 days, 4 days, 5 days, 6, days, 7 days, 8 days, 9 days, 10 days, 11 days, 12 days, 13 days, 14 days or 15 days. In other aspects of this embodiment, a composition disclosed herein is stable for, e.g., at least 1 day, at least 2 days, at least 3 days, at least 4 days, at least 5 days, at least 6, days, at least 7 days, at least 8 days, at least 9 days, at least 10 days, at least 11 days, at least 12 days, at least 13 days, at least 14 days or at least 15 days. In yet other aspects of this embodiment, a composition disclosed herein is stable for, e.g., at most 1 day, at most 2 days, at most 3 days, at most 4 days, at most 5 days, at most 6, days, at most 7 days, at most 8 days, at most 9 days, at most 10 days, at most 11 days, at most 12 days, at most 13 days, at most 14 days or at most 15 days.

In still aspects of this embodiment, a composition disclosed herein is stable for, e.g., about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 1 day to about 13 days, about 1 day to about 14 days, about 1 day to about 15 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 2 days to about 13 days, about 2 days to about 14 days, about 2 days to about 15 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 3 days to about 13 days, about 3 days to about 14 days, about 3 days to about 15 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 4 days to about 13 days, about 4 days to about 14 days, about 4 days to about 15 days, about 5 days to about 6 days, about 5 days to about 7 days, about 5 days to about 8 days, about 5 days to about 9 days, about 5 days to about 10 days, about 5 days to about 11 days, about 5 days to about 12 days, about 5 days to about 13 days, about 5 days to about 14 days, about 5 days to about 15 days, about 6 days to about 7 days, about 6 days to about 8 days, about 6 days to about 9 days, about 6 days to about 10 days, about 6 days to about 11 days, about 6 days to about 12 days, about 6 days to about 13 days, about 6 days to about 14 days, about 6 days to about 15 days, about 7 days to about 8 days, about 7 days to about 9 days, about 7 days to about 10 days, about 7 days to about 11 days, about 7 days to about 12 days, about 7 days to about 13 days, about 7 days to about 14 days, about 7 days to about 15 days, about 8 days to about 9 days, about 8 days to about 10 days, about 8 days to about 11 days, about 8 days to about 12 days, about 8 days to about 13 days, about 8 days to about 14 days, about 8 days to about 15 days, about 9 days to about 10 days, about 9 days to about 11 days, about 9 days to about 12 days, about 9 days to about 13 days, about 9 days to about 14 days, about 9 days to about 15 days, about 10 days to about 11 days, about 10 days to about 12 days, about 10 days to about 13 days, about 10 days to about 14 days, about 10 days to about 15 days, about 11 days to about 12 days, about 11 days to about 13 days, about 11 days to about 14 days, about 11 days to about 15 days, about 12 days to about 13 days, about 12 days to about 14 days, about 12 days to about 15 days, about 13 days to about 14 days, about 13 days to about 15 days, or about 14 days to about 15 days.

In other aspects of this embodiment, a composition disclosed herein is stable for, e.g., about 1 week, about 2 weeks, 3 weeks, 4 weeks, 5 weeks, 6, weeks, 7 weeks, 8 weeks, 9 weeks, 10 weeks, 11 weeks, 12 weeks, 13 weeks, 14 weeks or 15 weeks. In yet other aspects of this embodiment, a composition disclosed herein is stable for, e.g., at least 1 week, at least 2 weeks, at least 3 weeks, at least 4 weeks, at least 5 weeks, at least 6, weeks, at least 7 weeks, at least 8 weeks, at least 9 weeks, at least 10 weeks, at least 11 weeks, at least 12 weeks, at least 13 weeks, at least 14 weeks or at least 15 weeks. In still other aspects of this embodiment, a composition disclosed herein is stable for, e.g., at most 1 week, at most 2 weeks, at most 3 weeks, at most 4 weeks, at most 5 weeks, at most 6, weeks, at most 7 weeks, at most 8 weeks, at most 9 weeks, at most 10 weeks, at most 11 weeks, at most 12 weeks, at most 13 weeks, at most 14 weeks or at most 15 weeks.

In aspects of this embodiment, a composition disclosed herein is stable for, e.g., about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 1 week to about 13 weeks, about 1 week to about 14 weeks, about 1 week to about 15 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 2 weeks to about 13 weeks, about 2 weeks to about 14 weeks, about 2 weeks to about 15 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 3 weeks to about 13 weeks, about 3 weeks to about 14 weeks, about 3 weeks to about 15 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 4 weeks to about 13 weeks, about 4 weeks to about 14 weeks, about 4 weeks to about 15 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 5 weeks to about 13 weeks, about 5 weeks to about 14 weeks, about 5 weeks to about 15 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 6 weeks to about 13 weeks, about 6 weeks to about 14 weeks, about 6 weeks to about 15 weeks, about 7 weeks to about 8 weeks, about 7 weeks to about 9 weeks, about 7 weeks to about 10 weeks, about 7 weeks to about 11 weeks, about 7 weeks to about 12 weeks, about 7 weeks to about 13 weeks, about 7 weeks to about 14 weeks, about 7 weeks to about 15 weeks, about 8 weeks to about 9 weeks, about 8 weeks to about 10 weeks, about 8 weeks to about 11 weeks, about 8 weeks to about 12 weeks, about 8 weeks to about 13 weeks, about 8 weeks to about 14 weeks, about 8 weeks to about 15 weeks, about 9 weeks to about 10 weeks, about 9 weeks to about 11 weeks, about 9 weeks to about 12 weeks, about 9 weeks to about 13 weeks, about 9 weeks to about 14 weeks, about 9 weeks to about 15 weeks, about 10 weeks to about 11 weeks, about 10 weeks to about 12 weeks, about 10 weeks to about 13 weeks, about 10 weeks to about 14 weeks, about 10 weeks to about 15 weeks, about 11 weeks to about 12 weeks, about 11 weeks to about 13 weeks, about 11 weeks to about 14 weeks, about 11 weeks to about 15 weeks, about 12 weeks to about 13 weeks, about 12 weeks to about 14 weeks, about 12 weeks to about 15 weeks, about 13 weeks to about 14 weeks, about 13 weeks to about 15 weeks, or about 14 weeks to about 15 weeks.

In other aspects of this embodiment, a composition disclosed herein is stable for, e.g., about 1 month, about 2 months, 3 months, 4 months, 5 months, 6, months, 7 months, 8 months, 9 months, 10 months, 11 months, 12 months, 13 months, 14 months, 15 months, 16 months, 17 months, or 18 months. In yet other aspects of this embodiment, a composition disclosed herein is stable for, e.g., at least 1 month, at least 2 months, at least 3 months, at least 4 months, at least 5 months, at least 6, months, at least 7 months, at least 8 months, at least 9 months, at least 10 months, at least 11 months, at least 12 months, at least 13 months, at least 14 months, at least 15 months, at least 16 months, at least 17 months, or at least 18 months. In still other aspects of this embodiment, a composition disclosed herein is stable for, e.g., at most 1 month, at most 2 months, at most 3 months, at most 4 months, at most 5 months, at most 6, months, at most 7 months, at most 8 months, at most 9 months, at most 10 months, at most 11 months, at most 12 months, at most 13 months, at most 14 months, at most 15 months, at most 16 months, at most 17 months, or at most 18 months.

In aspects of this embodiment, a composition disclosed herein is stable for, e.g., about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 1 month to about 13 months, about 1 month to about 14 months, about 1 month to about 15 months, about 1 month to about 16 months, about 1 month to about 17 months, about 1 month to about 18 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 2 months to about 13 months, about 2 months to about 14 months, about 2 months to about 15 months, about 2 months to about 16 months, about 2 months to about 17 months, about 2 months to about 18 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 3 months to about 13 months, about 3 months to about 14 months, about 3 months to about 15 months, about 3 months to about 16 months, about 3 months to about 17 months, about 3 months to about 18 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 4 months to about 13 months, about 4 months to about 14 months, about 4 months to about 15 months, about 4 months to about 16 months, about 4 months to about 17 months, about 4 months to about 18 months, about 5 months to about 6 months, about 5 months to about 7 months, about 5 months to about 8 months, about 5 months to about 9 months, about 5 months to about 10 months, about 5 months to about 11 months, about 5 months to about 12 months, about 5 months to about 13 months, about 5 months to about 14 months, about 5 months to about 15 months, about 5 months to about 16 months, about 5 months to about 17 months, about 5 months to about 18 months, about 6 months to about 7 months, about 6 months to about 8 months, about 6 months to about 9 months, about 6 months to about 10 months, about 6 months to about 11 months, about 6 months to about 12 months, about 6 months to about 13 months, about 6 months to about 14 months, about 6 months to about 15 months, about 6 months to about 16 months, about 6 months to about 17 months, about 6 months to about 18 months, about 7 months to about 8 months, about 7 months to about 9 months, about 7 months to about 10 months, about 7 months to about 11 months, about 7 months to about 12 months, about 7 months to about 13 months, about 7 months to about 14 months, about 7 months to about 15 months, about 7 months to about 16 months, about 7 months to about 17 months, about 7 months to about 18 months, about 8 months to about 9 months, about 8 months to about 10 months, about 8 months to about 11 months, about 8 months to about 12 months, about 8 months to about 13 months, about 8 months to about 14 months, about 8 months to about 15 months, about 8 months to about 16 months, about 8 months to about 17 months, about 8 months to about 18 months, about 9 months to about 10 months, about 9 months to about 11 months, about 9 months to about 12 months, about 9 months to about 13 months, about 9 months to about 14 months, about 9 months to about 15 months, about 9 months to about 16 months, about 9 months to about 17 months, about 9 months to about 18 months, about 10 months to about 11 months, about 10 months to about 12 months, about 10 months to about 13 months, about 10 months to about 14 months, about 10 months to about 15 months, about 10 months to about 16 months, about 10 months to about 17 months, about 10 months to about 18 months, about 11 months to about 12 months, about 11 months to about 13 months, about 11 months to about 14 months, about 11 months to about 15 months, about 11 months to about 16 months, about 11 months to about 17 months, about 1 months to about 18 months, about 12 months to about 13 months, about 12 months to about 14 months, about 12 months to about 15 months, about 12 months to about 16 months, about 12 months to about 17 months, about 12 months to about 18 months, about 13 months to about 14 months, about 13 months to about 15 months, about 13 months to about 16 months, about 13 months to about 17 months, about 13 months to about 18 months, about 14 months to about 15 months, about 14 months to about 16 months, about 14 months to about 17 months, about 14 months to about 18 months, about 15 months to about 16 months, about 15 months to about 17 months, about 15 months to about 18 months, about 16 months to about 17 months, about 16 months to about 18 months, or about 17 months to about 18 months.

In other aspects of this embodiment, a composition disclosed herein is stable for, e.g., about 1 year, about 2 years, about 3 years, about 4 years, or about 5 years. In yet other aspects of this embodiment, a composition disclosed herein is stable for, e.g., at least 1 year, at least 2 years, at least 3 years, at least 4 years, or at least 5 years. In still other aspects of this embodiment, a composition disclosed herein is stable for, e.g., at most 1 year, at most 2 years, at most 3 years, at most 4 years, or at most 5 years. In other aspects of this embodiment, a composition disclosed herein is stable for, e.g., about 1 year to about 2 years, about 1 year to about 3 years, about 1 year to about 4 years, about 1 year to about 5 years, about 2 years to about 3 years, about 2 years to about 4 years, about 2 years to about 5 years, about 3 years to about 4 years, about 3 years to about 5 years, or about 4 years to about 5 years.

A composition disclosed herein can be formulated into any form that enables topical application of an anti-aging composition and/or a hair smoothing composition disclosed herein in a manner that achieves a desired beneficial effect. In one embodiment, a composition disclosed herein can be formulated into, e.g., a single-phase formulation, a biphasic formulation comprising a medium phase and a dispersed phase, or a multiphasic formulation. In another embodiment, a composition disclosed herein can be formulated into, e.g., a liquid composition, a colloidal composition, a semi-solid composition, or a solid composition. In another embodiment, a composition disclosed herein can be formulated into, e.g., a liquid aerosol, a foam, an emulsion, a gel, a sol, or a solid sol. In another embodiment, a composition disclosed herein can be formulated into, e.g., a spray, a liquid aerosol, a wash, an aftershave, a perfume, a body mist, a lotion, a cream, a salve, a waxing composition, a mousse, a shampoo, a conditioner, an ointment, a soap, a toner, a foundation, a stick, a film forming product, a suspension, or an emollient.

It is understood that a composition disclosed herein is useful for benefiting individuals from a variety of populations. Accordingly, a composition disclosed herein can be formulated based on the preferences of individuals from a target population. For example, individuals from North America are accustomed to thicker consistencies and heavier skin feel while individuals in China expect a thinner consistency and lighter skin feel. Accordingly, when formulating a composition disclosed herein regional expectations of an individual should be balanced with delivery of the desired skin and/or hair benefits.

In one embodiment, a composition disclosed herein comprises hypochlorous acid or free available chlorine and a silicone. In an aspect of this embodiment, a composition disclosed herein comprises about 0.1 ppm to about 300 ppm hypochlorous acid or free available chlorine and about 0.1% to about 20% of a silicone. In another aspect of this embodiment, a composition disclosed herein comprises about 0.25 ppm to about 250 ppm hypochlorous acid or free available chlorine and about 0.25% to about 18% of a silicone. In yet another aspect of this embodiment, a composition disclosed herein comprises about 0.75 ppm to about 200 ppm hypochlorous acid or free available chlorine and about 0.5% to about 15% of a silicone. In still another aspect of this embodiment, a composition disclosed herein comprises about 1 ppm to about 150 ppm hypochlorous acid or free available chlorine and about 0.75% to about 12% of a silicone. In another aspect of this embodiment, a composition disclosed herein comprises about 2.5 ppm to about 100 ppm hypochlorous acid or free available chlorine and about 1% to about 10% of a silicone. In yet another aspect of this embodiment, a composition disclosed herein comprises about 5 ppm to about 50 ppm hypochlorous acid or free available chlorine and about 1% to about 8% of a silicone.

In one embodiment, a composition disclosed herein comprises hypochlorous acid or free available chlorine and a silicone. In an aspect of this embodiment, a composition disclosed herein comprises about 15 ppm to about 40 ppm hypochlorous acid or free available chlorine and about 0.1% to about 10% of a silicone. In another aspect of this embodiment, a composition disclosed herein comprises about 18 ppm to about 35 ppm hypochlorous acid or free available chlorine and about 0.25% to about 8% of a silicone. In yet another aspect of this embodiment, a composition disclosed herein comprises about 20 ppm to about 33 ppm hypochlorous acid or free available chlorine and about 0.5% to about 7% of a silicone. In still another aspect of this embodiment, a composition disclosed herein comprises about 22 ppm to about 31 ppm hypochlorous acid or free available chlorine and about 0.75% to about 6% of a silicone. In another aspect of this embodiment, a composition disclosed herein comprises about 24 ppm to about 29 ppm hypochlorous acid or free available chlorine and about 1% to about 5% of a silicone.

In an aspect of this embodiment, a composition disclosed herein comprises about 15 ppm to about 40 ppm hypochlorous acid or free available chlorine and about 0.1% to about 10% of one or more silicones. In another aspect of this embodiment, a composition disclosed herein comprises about 18 ppm to about 35 ppm hypochlorous acid or free available chlorine and about 0.25% to about 8% of one or more silicones. In yet another aspect of this embodiment, a composition disclosed herein comprises about 20 ppm to about 33 ppm hypochlorous acid or free available chlorine and about 0.5% to about 7% of one or more silicones. In still another aspect of this embodiment, a composition disclosed herein comprises about 22 ppm to about 31 ppm hypochlorous acid or free available chlorine and about 0.75% to about 6% of one or more silicones. In another aspect of this embodiment, a composition disclosed herein comprises about 24 ppm to about 29 ppm hypochlorous acid or free available chlorine and about 1% to about 5% of one or more silicones.

In an aspect of this embodiment, a composition disclosed herein comprises about 15 ppm to about 40 ppm hypochlorous acid or free available chlorine and about 0.1% to about 10% of one or more silicone amines, one or more anionic silicones, or both one or more silicone amines and one or more anionic silicones. In another aspect of this embodiment, a composition disclosed herein comprises about 18 ppm to about 35 ppm hypochlorous acid or free available chlorine and about 0.25% to about 8% of one or more silicone amines, one or more anionic silicones, or both one or more silicone amines and one or more anionic silicones. In yet another aspect of this embodiment, a composition disclosed herein comprises about 20 ppm to about 33 ppm hypochlorous acid or free available chlorine and about 0.5% to about 7% of one or more silicone amines, one or more anionic silicones, or both one or more silicone amines and one or more anionic silicones. In still another aspect of this embodiment, a composition disclosed herein comprises about 22 ppm to about 31 ppm hypochlorous acid or free available chlorine and about 0.75% to about 6% of one or more silicone amines, one or more anionic silicones, or both one or more silicone amines and one or more anionic silicones. In another aspect of this embodiment, a composition disclosed herein comprises about 24 ppm to about 29 ppm hypochlorous acid or free available chlorine and about 1% to about 5% of one or more silicone amines, one or more anionic silicones, or both one or more silicone amines and one or more anionic silicones.

In an aspect of this embodiment, a composition disclosed herein comprises about 15 ppm to about 40 ppm hypochlorous acid or free available chlorine and about 0.1% to about 10% of a dimethicone, a dimethicone/vinyl dimethicone crosspolymer, amodimethicone, or any combination thereof. In another aspect of this embodiment, a composition disclosed herein comprises about 18 ppm to about 35 ppm hypochlorous acid or free available chlorine and about 0.25% to about 8% of a dimethicone, a dimethicone/vinyl dimethicone crosspolymer, amodimethicone, or any combination thereof. In yet another aspect of this embodiment, a composition disclosed herein comprises about 20 ppm to about 33 ppm hypochlorous acid or free available chlorine and about 0.5% to about 7% of a dimethicone, a dimethicone/vinyl dimethicone crosspolymer, amodimethicone, or any combination thereof. In still another aspect of this embodiment, a composition disclosed herein comprises about 22 ppm to about 31 ppm hypochlorous acid or free available chlorine and about 0.75% to about 6% of a dimethicone, a dimethicone/vinyl dimethicone crosspolymer, amodimethicone, or any combination thereof. In another aspect of this embodiment, a composition disclosed herein comprises about 24 ppm to about 29 ppm hypochlorous acid or free available chlorine and about 1% to about 5% of a dimethicone, a dimethicone/vinyl dimethicone crosspolymer, amodimethicone, or any combination thereof.

In an aspect of this embodiment, a composition disclosed herein comprises about 10 ppm to about 50 ppm hypochlorous acid or free available chlorine and about 0.1% to about 10% of a dimethicone, about 0.1% to about 10% of a dimethicone/vinyl dimethicone crosspolymer, about 0.003% to about 0.7% silica, and about 0.1% to about 10% of a silicate clay. In another aspect of this embodiment, a composition disclosed herein comprises about 15 ppm to about 45 ppm hypochlorous acid or free available chlorine and about 0.25% to about 8% of a dimethicone, about 0.25% to about 8% of a dimethicone/vinyl dimethicone crosspolymer, about 0.0.007% to about 0.6% silica, and about 0.25% to about 8% of a silicate clay. In yet another aspect of this embodiment, a composition disclosed herein comprises about 20 ppm to about 40 ppm hypochlorous acid or free available chlorine and about 0.5% to about 7% of a dimethicone, about 0.5% to about 7% of a dimethicone/vinyl dimethicone crosspolymer, about 0.01% to about 0.5% silica, and about 0.5% to about 7% of a silicate clay. In still another aspect of this embodiment, a composition disclosed herein comprises about 20 ppm to about 35 ppm hypochlorous acid or free available chlorine and about 0.5% to about 6% of a dimethicone, about 0.5% to about 6% of a dimethicone/vinyl dimethicone crosspolymer, about 0.02% to about 0.4% silica, and about 0.75% to about 6% of a silicate clay. In another aspect of this embodiment, a composition disclosed herein comprises about 25 ppm to about 30 ppm hypochlorous acid or free available chlorine and about 0.5% to about 5% of a dimethicone, about 0.5% to about 5% of a dimethicone/vinyl dimethicone crosspolymer, about 0.03% to about 0.3% silica, and about 1% to about 5% of a silicate clay. In further aspects of this embodiment, a composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, a composition disclosed herein comprises about 25 ppm to about 30 ppm hypochlorous acid or free available chlorine, about 0.6% to about 1.6% of a dimethicone, about 0.6% to about 1.6% of a dimethicone/vinyl dimethicone crosspolymer, about 0.018% to about 0.11% silica, and about 1.5% to about 2.5% of a silicate clay and has a pH of about 8.5 to about 8.7.

In an aspect of this embodiment, a composition disclosed herein comprises about 10 ppm to about 50 ppm hypochlorous acid or free available chlorine and about 0.1% to about 10% of a dimethicone, and about 0.1% to about 10% of a silicate clay. In another aspect of this embodiment, a composition disclosed herein comprises about 15 ppm to about 45 ppm hypochlorous acid or free available chlorine and about 0.25% to about 8% of a dimethicone, and about 0.25% to about 8% of a silicate clay. In yet another aspect of this embodiment, a composition disclosed herein comprises about 20 ppm to about 40 ppm hypochlorous acid or free available chlorine and about 0.5% to about 7% of a dimethicone, and about 0.5% to about 7% of a silicate clay. In still another aspect of this embodiment, a composition disclosed herein comprises about 20 ppm to about 35 ppm hypochlorous acid or free available chlorine and about 0.75% to about 6% of a dimethicone, and about 0.75% to about 6% of a silicate clay. In another aspect of this embodiment, a composition disclosed herein comprises about 25 ppm to about 30 ppm hypochlorous acid or free available chlorine and about 1% to about 5% of a dimethicone, and about 1% to about 5% of a silicate clay. In further aspects of this embodiment, a composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, a composition disclosed herein comprises about 25 ppm to about 30 ppm hypochlorous acid or free available chlorine, about 1.75% to about 2.75% of a dimethicone, and about 1.1% to about 2.1% of a silicate clay and has a pH of about 8.5 to about 8.7.

In another embodiment, an anti-aging composition disclosed herein comprises hypochlorous acid or free available chlorine and a silicone. In an aspect of this embodiment, an anti-aging composition disclosed herein comprises about 18 ppm to about 30 ppm hypochlorous acid or free available chlorine and about 0.1% to about 10% of a silicone. In another aspect of this embodiment, an anti-aging composition disclosed herein comprises about 20 ppm to about 28 ppm hypochlorous acid or free available chlorine and about 0.25% to about 8% of a silicone. In yet another aspect of this embodiment, an anti-aging composition disclosed herein comprises about 21 ppm to about 27 ppm hypochlorous acid or free available chlorine and about 0.5% to about 7% of a silicone. In still another aspect of this embodiment, an anti-aging composition disclosed herein comprises about 22 ppm to about 26 ppm hypochlorous acid or free available chlorine and about 0.75% to about 6% of a silicone. In another aspect of this embodiment, an anti-aging composition disclosed herein comprises about 23 ppm to about 25 ppm hypochlorous acid or free available chlorine and about 1% to about 5% of a silicone. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine and about 0.1% to about 10% of one or more silicones. In another aspect of this embodiment, an anti-aging composition disclosed herein comprises about 20 ppm to about 28 ppm hypochlorous acid or free available chlorine and about 0.25% to about 8% of one or more silicones. In yet another aspect of this embodiment, an anti-aging composition disclosed herein comprises about 21 ppm to about 27 ppm hypochlorous acid or free available chlorine and about 0.5% to about 7% of one or more silicones. In still another aspect of this embodiment, an anti-aging composition disclosed herein comprises about 22 ppm to about 26 ppm hypochlorous acid or free available chlorine and about 0.75% to about 6% of one or more silicones. In another aspect of this embodiment, an anti-aging composition disclosed herein comprises about 23 ppm to about 25 ppm hypochlorous acid or free available chlorine and about 1% to about 5% of one or more silicones. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine and about 0.1% to about 10% of one or more silicone amines, one or more anionic silicones, or both one or more silicone amines and one or more anionic silicones. In another aspect of this embodiment, an anti-aging composition disclosed herein comprises about 20 ppm to about 28 ppm hypochlorous acid or free available chlorine and about 0.25% to about 8% of one or more silicone amines, one or more anionic silicones, or both one or more silicone amines and one or more anionic silicones. In yet another aspect of this embodiment, an anti-aging composition disclosed herein comprises about 21 ppm to about 27 ppm hypochlorous acid or free available chlorine and about 0.5% to about 7% of one or more silicone amines, one or more anionic silicones, or both one or more silicone amines and one or more anionic silicones. In still another aspect of this embodiment, an anti-aging composition disclosed herein comprises about 22 ppm to about 26 ppm hypochlorous acid or free available chlorine and about 0.75% to about 6% of one or more silicone amines, one or more anionic silicones, or both one or more silicone amines and one or more anionic silicones. In another aspect of this embodiment, an anti-aging composition disclosed herein comprises about 23 ppm to about 25 ppm hypochlorous acid or free available chlorine and about 1% to about 5% of one or more silicone amines, one or more anionic silicones, or both one or more silicone amines and one or more anionic silicones. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine and about 0.1% to about 10% of a dimethicone, a dimethicone/vinyl dimethicone crosspolymer, amodimethicone, or any combination thereof. In another aspect of this embodiment, an anti-aging composition disclosed herein comprises about 20 ppm to about 28 ppm hypochlorous acid or free available chlorine and about 0.25% to about 8% of a dimethicone, a dimethicone/vinyl dimethicone crosspolymer, amodimethicone, or any combination thereof. In yet another aspect of this embodiment, an anti-aging composition disclosed herein comprises about 21 ppm to about 27 ppm hypochlorous acid or free available chlorine and about 0.5% to about 7% of a dimethicone, a dimethicone/vinyl dimethicone crosspolymer, amodimethicone, or any combination thereof. In still another aspect of this embodiment, an anti-aging composition disclosed herein comprises about 22 ppm to about 26 ppm hypochlorous acid or free available chlorine and about 0.75% to about 6% of a dimethicone, a dimethicone/vinyl dimethicone crosspolymer, amodimethicone, or any combination thereof. In another aspect of this embodiment, an anti-aging composition disclosed herein comprises about 23 ppm to about 25 ppm hypochlorous acid or free available chlorine and about 1% to about 5% of a dimethicone, a dimethicone/vinyl dimethicone crosspolymer, amodimethicone, or any combination thereof. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine and about 0.1% to about 10% of a silicone. In another aspect of this embodiment, an anti-aging composition disclosed herein comprises about 20 ppm to about 28 ppm hypochlorous acid or free available chlorine and about 1% to about 8% of a silicone. In yet another aspect of this embodiment, an anti-aging composition disclosed herein comprises about 21 ppm to about 27 ppm hypochlorous acid or free available chlorine and about 2% to about 7% of a silicone. In still another aspect of this embodiment, an anti-aging composition disclosed herein comprises about 22 ppm to about 26 ppm hypochlorous acid or free available chlorine and about 3% to about 6% of a silicone. In another aspect of this embodiment, an anti-aging composition disclosed herein comprises about 23 ppm to about 25 ppm hypochlorous acid or free available chlorine and about 4% to about 5% of a silicone. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine and about 0.1% to about 10% of one or more silicones. In another aspect of this embodiment, an anti-aging composition disclosed herein comprises about 20 ppm to about 28 ppm hypochlorous acid or free available chlorine and about 1% to about 8% of one or more silicones. In yet another aspect of this embodiment, an anti-aging composition disclosed herein comprises about 21 ppm to about 27 ppm hypochlorous acid or free available chlorine and about 2% to about 7% of one or more silicones. In still another aspect of this embodiment, an anti-aging composition disclosed herein comprises about 22 ppm to about 26 ppm hypochlorous acid or free available chlorine and about 3% to about 6% of one or more silicones. In another aspect of this embodiment, an anti-aging composition disclosed herein comprises about 23 ppm to about 25 ppm hypochlorous acid or free available chlorine and about 4% to about 5% of one or more silicones. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine and about 0.1% to about 10% of one or more silicone amines, one or more anionic silicones, or both one or more silicone amines and one or more anionic silicones. In another aspect of this embodiment, an anti-aging composition disclosed herein comprises about 20 ppm to about 28 ppm hypochlorous acid or free available chlorine and about 1% to about 8% of one or more silicone amines, one or more anionic silicones, or both one or more silicone amines and one or more anionic silicones. In yet another aspect of this embodiment, an anti-aging composition disclosed herein comprises about 21 ppm to about 27 ppm hypochlorous acid or free available chlorine and about 2% to about 7% of one or more silicone amines, one or more anionic silicones, or both one or more silicone amines and one or more anionic silicones. In still another aspect of this embodiment, an anti-aging composition disclosed herein comprises about 22 ppm to about 26 ppm hypochlorous acid or free available chlorine and about 3% to about 6% of one or more silicone amines, one or more anionic silicones, or both one or more silicone amines and one or more anionic silicones. In another aspect of this embodiment, an anti-aging composition disclosed herein comprises about 23 ppm to about 25 ppm hypochlorous acid or free available chlorine and about 4% to about 5% of one or more silicone amines, one or more anionic silicones, or both one or more silicone amines and one or more anionic silicones. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine and about 0.1% to about 10% of a dimethicone, a dimethicone/vinyl dimethicone crosspolymer, amodimethicone, or any combination thereof. In another aspect of this embodiment, an anti-aging composition disclosed herein comprises about 20 ppm to about 28 ppm hypochlorous acid or free available chlorine and about 1% to about 8% of a dimethicone, a dimethicone/vinyl dimethicone crosspolymer, amodimethicone, or any combination thereof. In yet another aspect of this embodiment, an anti-aging composition disclosed herein comprises about 21 ppm to about 27 ppm hypochlorous acid or free available chlorine and about 2% to about 7% of a dimethicone, a dimethicone/vinyl dimethicone crosspolymer, amodimethicone, or any combination thereof. In still another aspect of this embodiment, an anti-aging composition disclosed herein comprises about 22 ppm to about 26 ppm hypochlorous acid or free available chlorine and about 3% to about 6% of a dimethicone, a dimethicone/vinyl dimethicone crosspolymer, amodimethicone, or any combination thereof. In another aspect of this embodiment, an anti-aging composition disclosed herein comprises about 23 ppm to about 25 ppm hypochlorous acid or free available chlorine and about 4% to about 5% of a dimethicone, a dimethicone/vinyl dimethicone crosspolymer, amodimethicone, or any combination thereof. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In another aspect of this embodiment, an anti-aging composition disclosed herein comprises about 23 ppm to about 25 ppm hypochlorous acid or free available chlorine and about 4.5% to about 5% of an amodimethicone and has a pH of about 7.1 to about 7.3.

In an aspect of this embodiment, an anti-aging composition disclosed herein comprises about 10 ppm to about 50 ppm hypochlorous acid or free available chlorine and about 0.1% to about 10% of a dimethicone, about 0.1% to about 10% of a dimethicone/vinyl dimethicone crosspolymer, about 0.003% to about 0.7% silica, and about 0.1% to about 10% of a silicate clay. In another aspect of this embodiment, an anti-aging composition disclosed herein comprises about 15 ppm to about 45 ppm hypochlorous acid or free available chlorine and about 0.25% to about 8% of a dimethicone, about 0.25% to about 8% of a dimethicone/vinyl dimethicone crosspolymer, about 0.0.007% to about 0.6% silica, and about 0.25% to about 8% of a silicate clay. In yet another aspect of this embodiment, an anti-aging composition disclosed herein comprises about 20 ppm to about 40 ppm hypochlorous acid or free available chlorine and about 0.5% to about 7% of a dimethicone, about 0.5% to about 7% of a dimethicone/vinyl dimethicone crosspolymer, about 0.01% to about 0.5% silica, and about 0.5% to about 7% of a silicate clay. In still another aspect of this embodiment, an anti-aging composition disclosed herein comprises about 20 ppm to about 35 ppm hypochlorous acid or free available chlorine and about 0.5% to about 6% of a dimethicone, about 0.5% to about 6% of a dimethicone/vinyl dimethicone crosspolymer, about 0.02% to about 0.4% silica, and about 0.75% to about 6% of a silicate clay. In another aspect of this embodiment, an anti-aging composition disclosed herein comprises about 25 ppm to about 30 ppm hypochlorous acid or free available chlorine and about 0.5% to about 5% of a dimethicone, about 0.5% to about 5% of a dimethicone/vinyl dimethicone crosspolymer, about 0.03% to about 0.3% silica, and about 1% to about 5% of a silicate clay. In further aspects of this embodiment, an anti-aging composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, an anti-aging composition disclosed herein comprises about 25 ppm to about 30 ppm hypochlorous acid or free available chlorine, about 0.6% to about 1.6% of a dimethicone, about 0.6% to about 1.6% of a dimethicone/vinyl dimethicone crosspolymer, about 0.018% to about 0.11% silica, and about 1.5% to about 2.5% of a silicate clay and has a pH of about 8.5 to about 8.7.

In an aspect of this embodiment, a hair smoothing composition disclosed herein comprises about 10 ppm to about 50 ppm hypochlorous acid or free available chlorine and about 0.1% to about 10% of a dimethicone, and about 0.1% to about 10% of a silicate clay. In another aspect of this embodiment, a hair smoothing composition disclosed herein comprises about 15 ppm to about 45 ppm hypochlorous acid or free available chlorine and about 0.25% to about 8% of a dimethicone, and about 0.25% to about 8% of a silicate clay. In yet another aspect of this embodiment, a hair smoothing composition disclosed herein comprises about 20 ppm to about 40 ppm hypochlorous acid or free available chlorine and about 0.5% to about 7% of a dimethicone, and about 0.5% to about 7% of a silicate clay. In still another aspect of this embodiment, a hair smoothing composition disclosed herein comprises about 20 ppm to about 35 ppm hypochlorous acid or free available chlorine and about 0.75% to about 6% of a dimethicone, and about 0.75% to about 6% of a silicate clay. In another aspect of this embodiment, a hair smoothing composition disclosed herein comprises about 25 ppm to about 30 ppm hypochlorous acid or free available chlorine and about 1% to about 5% of a dimethicone, and about 1% to about 5% of a silicate clay. In further aspects of this embodiment, a hair smoothing composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, a hair smoothing composition disclosed herein comprises about 25 ppm to about 30 ppm hypochlorous acid or free available chlorine, about 1.75% to about 2.75% of a dimethicone, and about 1.1% to about 2.1% of a silicate clay and has a pH of about 8.5 to about 8.7.

In another embodiment, a hair smoothing composition disclosed herein comprises hypochlorous acid or free available chlorine and a silicone. In an aspect of this embodiment, a hair smoothing composition disclosed herein comprises about 23 ppm to about 35 ppm hypochlorous acid or free available chlorine and about 0.1% to about 10% of a silicone. In another aspect of this embodiment, a hair smoothing composition disclosed herein comprises about 25 ppm to about 33 ppm hypochlorous acid or free available chlorine and about 2% to about 8% of a silicone. In yet another aspect of this embodiment, a hair smoothing composition disclosed herein comprises about 26 ppm to about 32 ppm hypochlorous acid or free available chlorine and about 3% to about 7% of a silicone. In still another aspect of this embodiment, a hair smoothing composition disclosed herein comprises about 27 ppm to about 31 ppm hypochlorous acid or free available chlorine and about 0.75% to 6% of a silicone. In another aspect of this embodiment, a hair smoothing composition disclosed herein comprises about 28 ppm to about 30 ppm hypochlorous acid or free available chlorine and about 4% to about 5% of a silicone. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

In an aspect of this embodiment, a hair smoothing composition disclosed herein comprises about 23 ppm to about 35 ppm hypochlorous acid or free available chlorine and about 0.1% to about 10% of one or more silicones. In another aspect of this embodiment, a hair smoothing composition disclosed herein comprises about 25 ppm to about 33 ppm hypochlorous acid or free available chlorine and about 1% to about 8% of one or more silicones. In yet another aspect of this embodiment, a hair smoothing composition disclosed herein comprises about 26 ppm to about 32 ppm hypochlorous acid or free available chlorine and about 2% to about 7% of one or more silicones. In still another aspect of this embodiment, a hair smoothing composition disclosed herein comprises about 27 ppm to about 31 ppm hypochlorous acid or free available chlorine and about 3% to about 6% of one or more silicones. In another aspect of this embodiment, a hair smoothing composition disclosed herein comprises about 28 ppm to about 30 ppm hypochlorous acid or free available chlorine and about 4% to about 5% of one or more silicones. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

In an aspect of this embodiment, a hair smoothing composition disclosed herein comprises about 23 ppm to about 35 ppm hypochlorous acid or free available chlorine and about 0.1% to about 10% of one or more silicone amines, one or more anionic silicones, or both one or more silicone amines and one or more anionic silicones. In another aspect of this embodiment, a hair smoothing composition disclosed herein comprises about 25 ppm to about 33 ppm hypochlorous acid or free available chlorine and about 1% to about 8% of one or more silicone amines, one or more anionic silicones, or both one or more silicone amines and one or more anionic silicones. In yet another aspect of this embodiment, a hair smoothing composition disclosed herein comprises about 26 ppm to about 32 ppm hypochlorous acid or free available chlorine and about 2% to about 7% of one or more silicone amines, one or more anionic silicones, or both one or more silicone amines and one or more anionic silicones. In still another aspect of this embodiment, a hair smoothing composition disclosed herein comprises about 27 ppm to about 31 ppm hypochlorous acid or free available chlorine and about 3% to about 6% of one or more silicone amines, one or more anionic silicones, or both one or more silicone amines and one or more anionic silicones. In another aspect of this embodiment, a hair smoothing composition disclosed herein comprises about 28 ppm to about 30 ppm hypochlorous acid or free available chlorine and about 4% to about 5% of one or more silicone amines, one or more anionic silicones, or both one or more silicone amines and one or more anionic silicones. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

In an aspect of this embodiment, a hair smoothing composition disclosed herein comprises about 23 ppm to about 35 ppm hypochlorous acid or free available chlorine and about 0.1% to about 10% of a dimethicone, a dimethicone/vinyl dimethicone crosspolymer, amodimethicone, or any combination thereof. In another aspect of this embodiment, a hair smoothing composition disclosed herein comprises about 25 ppm to about 33 ppm hypochlorous acid or free available chlorine and about 1% to about 8% of a dimethicone, a dimethicone/vinyl dimethicone crosspolymer, amodimethicone, or any combination thereof. In yet another aspect of this embodiment, a hair smoothing composition disclosed herein comprises about 26 ppm to about 32 ppm hypochlorous acid or free available chlorine and about 2% to about 7% of a dimethicone, a dimethicone/vinyl dimethicone crosspolymer, amodimethicone, or any combination thereof. In still another aspect of this embodiment, a hair smoothing composition disclosed herein comprises about 27 ppm to about 31 ppm hypochlorous acid or free available chlorine and about 3% to about 6% of a dimethicone, a dimethicone/vinyl dimethicone crosspolymer, amodimethicone, or any combination thereof. In another aspect of this embodiment, a hair smoothing composition disclosed herein comprises about 28 ppm to about 30 ppm hypochlorous acid or free available chlorine and about 4% to about 5% of a dimethicone, a dimethicone/vinyl dimethicone crosspolymer, amodimethicone, or any combination thereof. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

In another aspect of this embodiment, a hair smoothing composition disclosed herein comprises about 28 ppm to about 30 ppm hypochlorous acid or free available chlorine and about 4.5% to about 5% of a dimethicone or amodimethicone and has a pH of about 5.6 to about 5.8.

In one embodiment, a composition disclosed herein comprises hypochlorous acid or free available chlorine, silicone and a rheology agent. In an aspect of this embodiment, a composition disclosed herein comprises about 15 ppm to about 40 ppm hypochlorous acid or free available chlorine, about 0.1% to about 10% of a silicone, and about 0.1% to about 7% of a rheology agent. In another aspect of this embodiment, a composition disclosed herein comprises about 18 ppm to about 35 ppm hypochlorous acid or free available chlorine, about 0.25% to about 8% of a silicone, and about 0.25% to about 5% of a rheology agent. In yet another aspect of this embodiment, a composition disclosed herein comprises about 20 ppm to about 33 ppm hypochlorous acid or free available chlorine, about 0.5% to about 7% of a silicone, and about 0.5% to about 4% of a rheology agent. In still another aspect of this embodiment, a composition disclosed herein comprises about 22 ppm to about 31 ppm hypochlorous acid or free available chlorine, about 0.75% to about 6% of a silicone, and about 0.75% to about 3% of a rheology agent. In another aspect of this embodiment, a composition disclosed herein comprises about 24 ppm to about 29 ppm hypochlorous acid or free available chlorine, about 1% to about 5% of a silicone, and about 1% to about 2% of a rheology agent.

In an aspect of this embodiment, a composition disclosed herein comprises about 15 ppm to about 40 ppm hypochlorous acid or free available chlorine, about 0.1% to about 10% of one or more silicones, and about 0.1% to about 7% of one or more rheology agents. In another aspect of this embodiment, a composition disclosed herein comprises about 18 ppm to about 35 ppm hypochlorous acid or free available chlorine, about 0.25% to about 8% of one or more silicones, and about 0.25% to about 5% of one or more rheology agents. In yet another aspect of this embodiment, a composition disclosed herein comprises about 20 ppm to about 33 ppm hypochlorous acid or free available chlorine, about 0.5% to about 7% of one or more silicones, and about 0.5% to about 4% of one or more rheology agents. In still another aspect of this embodiment, a composition disclosed herein comprises about 22 ppm to about 31 ppm hypochlorous acid or free available chlorine, about 0.75% to about 6% of one or more silicones, and about 0.75% to about 3% of one or more rheology agents. In another aspect of this embodiment, a composition disclosed herein comprises about 24 ppm to about 29 ppm hypochlorous acid or free available chlorine, about 1% to about 5% of one or more silicones, and about 1% to about 2% of one or more rheology agents.

In an aspect of this embodiment, a composition disclosed herein comprises about 15 ppm to about 40 ppm hypochlorous acid or free available chlorine, about 0.1% to about 10% of one or more silicone amines, one or more anionic silicones, or both one or more silicone amines and one or more anionic silicones, and about 0.1% to about 7% of one or more rheology agents. In another aspect of this embodiment, a composition disclosed herein comprises about 18 ppm to about 35 ppm hypochlorous acid or free available chlorine, about 0.25% to about 8% of one or more silicone amines, one or more anionic silicones, or both one or more silicone amines and one or more anionic silicones, and about 0.25% to about 5% of one or more rheology agents. In yet another aspect of this embodiment, a composition disclosed herein comprises about 20 ppm to about 33 ppm hypochlorous acid or free available chlorine, about 0.5% to about 7% of one or more silicone amines, one or more anionic silicones, or both one or more silicone amines and one or more anionic silicones, and about 0.5% to about 4% of one or more rheology agents. In still another aspect of this embodiment, a composition disclosed herein comprises about 22 ppm to about 31 ppm hypochlorous acid or free available chlorine, about 0.75% to about 6% of one or more silicone amines, one or more anionic silicones, or both one or more silicone amines and one or more anionic silicones, and about 0.75% to about 3% of one or more rheology agents. In another aspect of this embodiment, a composition disclosed herein comprises about 24 ppm to about 29 ppm hypochlorous acid or free available chlorine, about 1% to about 5% of one or more silicone amines, one or more anionic silicones, or both one or more silicone amines and one or more anionic silicones, and about 1% to about 2% of one or more rheology agents.

In an aspect of this embodiment, a composition disclosed herein comprises about 15 ppm to about 40 ppm hypochlorous acid or free available chlorine, about 0.1% to about 10% of a dimethicone, a dimethicone/vinyl dimethicone crosspolymer, amodimethicone, or any combination thereof, and about 0.1% to about 7% of one or more rheology agents. In another aspect of this embodiment, a composition disclosed herein comprises about 18 ppm to about 35 ppm hypochlorous acid or free available chlorine, about 0.25% to about 8% of a dimethicone, a dimethicone/vinyl dimethicone crosspolymer, amodimethicone, or any combination thereof, and about 0.25% to about 5% of one or more rheology agents. In yet another aspect of this embodiment, a composition disclosed herein comprises about 20 ppm to about 33 ppm hypochlorous acid or free available chlorine, about 0.5% to about 7% of a dimethicone, a dimethicone/vinyl dimethicone crosspolymer, amodimethicone, or any combination thereof, and about 0.5% to about 4% of one or more rheology agents. In still another aspect of this embodiment, a composition disclosed herein comprises about 22 ppm to about 31 ppm hypochlorous acid or free available chlorine, about 0.75% to about 6% of a dimethicone, a dimethicone/vinyl dimethicone crosspolymer, amodimethicone, or any combination thereof, and about 0.75% to about 3% of one or more rheology agents. In another aspect of this embodiment, a composition disclosed herein comprises about 24 ppm to about 29 ppm hypochlorous acid or free available chlorine, about 1% to about 5% of a dimethicone, a dimethicone/vinyl dimethicone crosspolymer, amodimethicone, or any combination thereof, and about 1% to about 2% of one or more rheology agents.

In another embodiment, an anti-aging composition disclosed herein comprises hypochlorous acid or free available chlorine, silicone and a rheology agent. In an aspect of this embodiment, an anti-aging composition disclosed herein comprises about 18 ppm to about 30 ppm hypochlorous acid or free available chlorine, about 0.1% to about 10% of a silicone, and about 0.1% to about 7% of a rheology agent. In another aspect of this embodiment, an anti-aging composition disclosed herein comprises about 20 ppm to about 28 ppm hypochlorous acid or free available chlorine, about 0.25% to about 8% of a silicone, and about 0.25% to about 5% of a rheology agent. In yet another aspect of this embodiment, an anti-aging composition disclosed herein comprises about 21 ppm to about 27 ppm hypochlorous acid or free available chlorine, about 0.5% to about 7% of a silicone, and about 0.5% to about 4% of a rheology agent. In still another aspect of this embodiment, an anti-aging composition disclosed herein comprises about 22 ppm to about 26 ppm hypochlorous acid or free available chlorine, about 0.75% to about 6% of a silicone, and about 0.75% to about 3% of a rheology agent. In another aspect of this embodiment, an anti-aging composition disclosed herein comprises about 23 ppm to about 25 ppm hypochlorous acid or free available chlorine, about 1% to about 5% of a silicone, and about 1% to about 2% of a rheology agent. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine, about 0.1% to about 10% of one or more silicones, and about 0.1% to about 7% of one or more rheology agents. In another aspect of this embodiment, an anti-aging composition disclosed herein comprises about 20 ppm to about 28 ppm hypochlorous acid or free available chlorine, about 0.25% to about 8% of one or more silicones, and about 0.25% to about 5% of one or more rheology agents. In yet another aspect of this embodiment, an anti-aging composition disclosed herein comprises about 21 ppm to about 27 ppm hypochlorous acid or free available chlorine, about 0.5% to about 7% of one or more silicones, and about 0.5% to about 4% of one or more rheology agents. In still another aspect of this embodiment, an anti-aging composition disclosed herein comprises about 22 ppm to about 26 ppm hypochlorous acid or free available chlorine, about 0.75% to about 6% of one or more silicones, and about 0.75% to about 3% of one or more rheology agents. In another aspect of this embodiment, an anti-aging composition disclosed herein comprises about 23 ppm to about 25 ppm hypochlorous acid or free available chlorine, about 1% to about 5% of one or more silicones, and about 1% to about 2% of one or more rheology agents. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine, about 0.1% to about 10% of one or more silicone amines, one or more anionic silicones, or both one or more silicone amines and one or more anionic silicones, and about 0.1% to about 7% of one or more rheology agents. In another aspect of this embodiment, an anti-aging composition disclosed herein comprises about 20 ppm to about 28 ppm hypochlorous acid or free available chlorine, about 0.25% to about 8% of one or more silicone amines, one or more anionic silicones, or both one or more silicone amines and one or more anionic silicones, and about 0.25% to about 5% of one or more rheology agents. In yet another aspect of this embodiment, an anti-aging composition disclosed herein comprises about 21 ppm to about 27 ppm hypochlorous acid or free available chlorine, about 0.5% to about 7% of one or more silicone amines, one or more anionic silicones, or both one or more silicone amines and one or more anionic silicones, and about 0.5% to about 4% of one or more rheology agents. In still another aspect of this embodiment, an anti-aging composition disclosed herein comprises about 22 ppm to about 26 ppm hypochlorous acid or free available chlorine, about 0.75% to about 6% of one or more silicone amines, one or more anionic silicones, or both one or more silicone amines and one or more anionic silicones, and about 0.75% to about 3% of one or more rheology agents. In another aspect of this embodiment, an anti-aging composition disclosed herein comprises about 23 ppm to about 25 ppm hypochlorous acid or free available chlorine, about 1% to about 5% of one or more silicone amines, one or more anionic silicones, or both one or more silicone amines and one or more anionic silicones, and about 1% to about 2% of one or more rheology agents. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine, about 0.1% to about 10% of a dimethicone, a dimethicone/vinyl dimethicone crosspolymer, amodimethicone, or any combination thereof, and about 0.1% to about 7% of one or more rheology agents. In another aspect of this embodiment, an anti-aging composition disclosed herein comprises about 20 ppm to about 28 ppm hypochlorous acid or free available chlorine, about 0.25% to about 8% of a dimethicone, a dimethicone/vinyl dimethicone crosspolymer, amodimethicone, or any combination thereof, and about 0.25% to about 5% of one or more rheology agents. In yet another aspect of this embodiment, an anti-aging composition disclosed herein comprises about 21 ppm to about 27 ppm hypochlorous acid or free available chlorine, about 0.5% to about 7% of a dimethicone, a dimethicone/vinyl dimethicone crosspolymer, amodimethicone, or any combination thereof, and about 0.5% to about 4% of one or more rheology agents. In still another aspect of this embodiment, an anti-aging composition disclosed herein comprises about 22 ppm to about 26 ppm hypochlorous acid or free available chlorine, about 0.75% to about 6% of a dimethicone, a dimethicone/vinyl dimethicone crosspolymer, amodimethicone, or any combination thereof, and about 0.75% to about 3% of one or more rheology agents. In another aspect of this embodiment, an anti-aging composition disclosed herein comprises about 23 ppm to about 25 ppm hypochlorous acid or free available chlorine, about 1% to about 5% of a dimethicone, a dimethicone/vinyl dimethicone crosspolymer, amodimethicone, or any combination thereof, and about 1% to about 2% of one or more rheology agents. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine, about 0.1% to about 5% of a silicone, and about 0.1% to about 7% of a rheology agent. In another aspect of this embodiment, an anti-aging composition disclosed herein comprises about 20 ppm to about 28 ppm hypochlorous acid or free available chlorine, about 0.15% to about 3% of a silicone, and about 0.25% to about 5% of a rheology agent. In yet another aspect of this embodiment, an anti-aging composition disclosed herein comprises about 21 ppm to about 27 ppm hypochlorous acid or free available chlorine, about 0.25% to about 2% of a silicone, and about 0.5% to about 4% of a rheology agent. In still another aspect of this embodiment, an anti-aging composition disclosed herein comprises about 22 ppm to about 26 ppm hypochlorous acid or free available chlorine, about 0.5% to about 1.5% of a silicone, and about 0.75% to about 3% of a rheology agent. In another aspect of this embodiment, an anti-aging composition disclosed herein comprises about 23 ppm to about 25 ppm hypochlorous acid or free available chlorine, about 0.75% to about 1.25% of a silicone, and about 1% to about 2% of a rheology agent. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine, about 0.1% to about 5% of one or more silicones, and about 0.1% to about 7% of one or more rheology agents. In another aspect of this embodiment, an anti-aging composition disclosed herein comprises about 20 ppm to about 28 ppm hypochlorous acid or free available chlorine, about 0.15% to about 3% of one or more silicones, and about 0.25% to about 5% of one or more rheology agents. In yet another aspect of this embodiment, an anti-aging composition disclosed herein comprises about 21 ppm to about 27 ppm hypochlorous acid or free available chlorine, about 0.25% to about 2% of one or more silicones, and about 0.5% to about 4% of one or more rheology agents. In still another aspect of this embodiment, an anti-aging composition disclosed herein comprises about 22 ppm to about 26 ppm hypochlorous acid or free available chlorine, about 0.5% to about 1.5% of one or more silicones, and about 0.75% to about 3% of one or more rheology agents. In another aspect of this embodiment, an anti-aging composition disclosed herein comprises about 23 ppm to about 25 ppm hypochlorous acid or free available chlorine, about 0.75% to about 1.25% of one or more silicones, and about 1% to about 2% of one or more rheology agents. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine, about 0.1% to about 5% of one or more silicone amines, one or more anionic silicones, or both one or more silicone amines and one or more anionic silicones, and about 0.1% to about 7% of one or more rheology agents. In another aspect of this embodiment, an anti-aging composition disclosed herein comprises about 20 ppm to about 28 ppm hypochlorous acid or free available chlorine, about 0.15% to about 3% of one or more silicone amines, one or more anionic silicones, or both one or more silicone amines and one or more anionic silicones, and about 0.25% to about 5% of one or more rheology agents. In yet another aspect of this embodiment, an anti-aging composition disclosed herein comprises about 21 ppm to about 27 ppm hypochlorous acid or free available chlorine, about 0.25% to about 2% of one or more silicone amines, one or more anionic silicones, or both one or more silicone amines and one or more anionic silicones, and about 0.5% to about 4% of one or more rheology agents. In still another aspect of this embodiment, an anti-aging composition disclosed herein comprises about 22 ppm to about 26 ppm hypochlorous acid or free available chlorine, about 0.5% to about 1.5% of one or more silicone amines, one or more anionic silicones, or both one or more silicone amines and one or more anionic silicones, and about 0.75% to about 3% of one or more rheology agents. In another aspect of this embodiment, an anti-aging composition disclosed herein comprises about 23 ppm to about 25 ppm hypochlorous acid or free available chlorine, about 0.75% to about 1.25% of one or more silicone amines, one or more anionic silicones, or both one or more silicone amines and one or more anionic silicones, and about 1% to about 2% of one or more rheology agents. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 10 ppm to about 50 ppm hypochlorous acid or free available chlorine and about 0.1% to about 10% of a dimethicone, about 0.1% to about 10% of a dimethicone/vinyl dimethicone crosspolymer, about 0.003% to about 0.7% silica, and about 0.1% to about 10% of one or more rheology agents. In another aspect of this embodiment, an anti-aging composition disclosed herein comprises about 15 ppm to about 45 ppm hypochlorous acid or free available chlorine and about 0.25% to about 8% of a dimethicone, about 0.25% to about 8% of a dimethicone/vinyl dimethicone crosspolymer, about 0.0.007% to about 0.6% silica, and about 0.25% to about 8% of one or more rheology agents. In yet another aspect of this embodiment, an anti-aging composition disclosed herein comprises about 20 ppm to about 40 ppm hypochlorous acid or free available chlorine and about 0.5% to about 7% of a dimethicone, about 0.5% to about 7% of a dimethicone/vinyl dimethicone crosspolymer, about 0.01% to about 0.5% silica, and about 0.5% to about 7% of one or more rheology agents. In still another aspect of this embodiment, an anti-aging composition disclosed herein comprises about 20 ppm to about 35 ppm hypochlorous acid or free available chlorine and about 0.5% to about 6% of a dimethicone, about 0.5% to about 6% of a dimethicone/vinyl dimethicone crosspolymer, about 0.02% to about 0.4% silica, and about 0.75% to about 6% of one or more rheology agents. In another aspect of this embodiment, an anti-aging composition disclosed herein comprises about 25 ppm to about 30 ppm hypochlorous acid or free available chlorine and about 0.5% to about 5% of a dimethicone, about 0.5% to about 5% of a dimethicone/vinyl dimethicone crosspolymer, about 0.03% to about 0.3% silica, and about 1% to about 5% of one or more rheology agents. In further aspects of this embodiment, an anti-aging composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, an anti-aging composition disclosed herein comprises about 25 ppm to about 30 ppm hypochlorous acid or free available chlorine, about 0.6% to about 1.6% of a dimethicone, about 0.6% to about 1.6% of a dimethicone/vinyl dimethicone crosspolymer, about 0.018% to about 0.11% silica, and about 1.5% to about 2.5% of one or more rheology agents and has a pH of about 8.5 to about 8.7.

In an aspect of this embodiment, a hair smoothing composition disclosed herein comprises about 10 ppm to about 50 ppm hypochlorous acid or free available chlorine and about 0.1% to about 10% of a dimethicone, and about 0.1% to about 10% of one or more rheology agents. In another aspect of this embodiment, an anti-aging composition disclosed herein comprises about 15 ppm to about 45 ppm hypochlorous acid or free available chlorine and about 0.25% to about 8% of a dimethicone, and about 0.25% to about 8% of one or more rheology agents. In yet another aspect of this embodiment, a hair smoothing composition disclosed herein comprises about 20 ppm to about 40 ppm hypochlorous acid or free available chlorine and about 0.5% to about 7% of a dimethicone, and about 0.5% to about 7% of one or more rheology agents. In still another aspect of this embodiment, a hair smoothing composition disclosed herein comprises about 20 ppm to about 35 ppm hypochlorous acid or free available chlorine and about 0.75% to about 6% of a dimethicone, and about 0.75% to about 6% of one or more rheology agents. In another aspect of this embodiment, a hair smoothing composition disclosed herein comprises about 25 ppm to about 30 ppm hypochlorous acid or free available chlorine and about 1% to about 5% of a dimethicone, and about 1% to about 5% of one or more rheology agents. In further aspects of this embodiment, a hair smoothing composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, a hair smoothing composition disclosed herein comprises about 25 ppm to about 30 ppm hypochlorous acid or free available chlorine, about 1.75% to about 2.75% of a dimethicone, and about 1.1% to about 2.1% of one or more rheology agents and has a pH of about 8.5 to about 8.7.

In an aspect of this embodiment, an anti-aging composition disclosed herein comprises about 10 ppm to about 50 ppm hypochlorous acid or free available chlorine and about 0.1% to about 10% of a dimethicone, about 0.1% to about 10% of a dimethicone/vinyl dimethicone crosspolymer, about 0.003% to about 0.7% silica, and about 0.1% to about 7% of one or more rheology agents. In another aspect of this embodiment, an anti-aging composition disclosed herein comprises about 15 ppm to about 45 ppm hypochlorous acid or free available chlorine and about 0.25% to about 8% of a dimethicone, about 0.25% to about 8% of a dimethicone/vinyl dimethicone crosspolymer, about 0.0.007% to about 0.6% silica, and about 0.25% to about 5% of one or more rheology agents. In yet another aspect of this embodiment, an anti-aging composition disclosed herein comprises about 20 ppm to about 40 ppm hypochlorous acid or free available chlorine and about 0.5% to about 7% of a dimethicone, about 0.5% to about 7% of a dimethicone/vinyl dimethicone crosspolymer, about 0.01% to about 0.5% silica, and about 0.5% to about 4% of one or more rheology agents. In still another aspect of this embodiment, an anti-aging composition disclosed herein comprises about 20 ppm to about 35 ppm hypochlorous acid or free available chlorine and about 0.5% to about 6% of a dimethicone, about 0.5% to about 6% of a dimethicone/vinyl dimethicone crosspolymer, about 0.02% to about 0.4% silica, and about 0.75% to about 3% of one or more rheology agents. In another aspect of this embodiment, an anti-aging composition disclosed herein comprises about 25 ppm to about 30 ppm hypochlorous acid or free available chlorine and about 0.5% to about 5% of a dimethicone, about 0.5% to about 5% of a dimethicone/vinyl dimethicone crosspolymer, about 0.03% to about 0.3% silica, and about 1% to about 2% of one or more rheology agents. In further aspects of this embodiment, an anti-aging composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, an anti-aging composition disclosed herein comprises about 25 ppm to about 30 ppm hypochlorous acid or free available chlorine, about 0.6% to about 1.6% of a dimethicone, about 0.6% to about 1.6% of a dimethicone/vinyl dimethicone crosspolymer, about 0.018% to about 0.11% silica, and about 1% to about 2% of one or more rheology agents and has a pH of about 8.5 to about 8.7.

In an aspect of this embodiment, a hair smoothing composition disclosed herein comprises about 10 ppm to about 50 ppm hypochlorous acid or free available chlorine and about 0.1% to about 10% of a dimethicone, and about 0.1% to about 7% of one or more rheology agents. In another aspect of this embodiment, a hair smoothing composition disclosed herein comprises about 15 ppm to about 45 ppm hypochlorous acid or free available chlorine and about 0.25% to about 8% of a dimethicone, and about 0.25% to about 5% of one or more rheology agents. In yet another aspect of this embodiment, a hair smoothing composition disclosed herein comprises about 20 ppm to about 40 ppm hypochlorous acid or free available chlorine and about 0.5% to about 7% of a dimethicone, and about 0.5% to about 4% of one or more rheology agents. In still another aspect of this embodiment, a hair smoothing composition disclosed herein comprises about 20 ppm to about 35 ppm hypochlorous acid or free available chlorine and about 0.75% to about 6% of a dimethicone, and about 0.75% to about 3% of one or more rheology agents. In another aspect of this embodiment, a hair smoothing composition disclosed herein comprises about 25 ppm to about 30 ppm hypochlorous acid or free available chlorine and about 1% to about 5% of a dimethicone, and about 1% to about 2% of one or more rheology agents. In further aspects of this embodiment, a hair smoothing composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, a hair smoothing composition disclosed herein comprises about 25 ppm to about 30 ppm hypochlorous acid or free available chlorine, about 1.75% to about 2.75% of a dimethicone, and about 1% to about 2% of one or more rheology agents and has a pH of about 8.5 to about 8.7.

In an aspect of this embodiment, an anti-aging composition disclosed herein comprises about 10 ppm to about 50 ppm hypochlorous acid or free available chlorine and about 0.1% to about 10% of a dimethicone, about 0.1% to about 10% of a dimethicone/vinyl dimethicone crosspolymer, about 0.003% to about 0.7% silica, and about 0.1% to about 10% of a silicate clay. In another aspect of this embodiment, an anti-aging composition disclosed herein comprises about 15 ppm to about 45 ppm hypochlorous acid or free available chlorine and about 0.25% to about 8% of a dimethicone, about 0.25% to about 8% of a dimethicone/vinyl dimethicone crosspolymer, about 0.0.007% to about 0.6% silica, and about 0.25% to about 8% of a silicate clay. In yet another aspect of this embodiment, an anti-aging composition disclosed herein comprises about 20 ppm to about 40 ppm hypochlorous acid or free available chlorine and about 0.5% to about 7% of a dimethicone, about 0.5% to about 7% of a dimethicone/vinyl dimethicone crosspolymer, about 0.01% to about 0.5% silica, and about 0.5% to about 7% of a silicate clay. In still another aspect of this embodiment, an anti-aging composition disclosed herein comprises about 20 ppm to about 35 ppm hypochlorous acid or free available chlorine and about 0.5% to about 6% of a dimethicone, about 0.5% to about 6% of a dimethicone/vinyl dimethicone crosspolymer, about 0.02% to about 0.4% silica, and about 0.75% to about 6% of a silicate clay. In another aspect of this embodiment, an anti-aging composition disclosed herein comprises about 25 ppm to about 30 ppm hypochlorous acid or free available chlorine and about 0.5% to about 5% of a dimethicone, about 0.5% to about 5% of a dimethicone/vinyl dimethicone crosspolymer, about 0.03% to about 0.3% silica, and about 1% to about 5% of a silicate clay. In further aspects of this embodiment, an anti-aging composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, an anti-aging composition disclosed herein comprises about 25 ppm to about 30 ppm hypochlorous acid or free available chlorine, about 0.6% to about 1.6% of a dimethicone, about 0.6% to about 1.6% of a dimethicone/vinyl dimethicone crosspolymer, and about 1.5% to about 2.5% of a silicate clay and has a pH of about 8.5 to about 8.7.

In an aspect of this embodiment, a hair smoothing composition disclosed herein comprises about 10 ppm to about 50 ppm hypochlorous acid or free available chlorine and about 0.1% to about 10% of a dimethicone, and about 0.1% to about 10% of a silicate clay. In another aspect of this embodiment, a hair smoothing composition disclosed herein comprises about 15 ppm to about 45 ppm hypochlorous acid or free available chlorine and about 0.25% to about 8% of a dimethicone, and about 0.25% to about 8% of a silicate clay. In yet another aspect of this embodiment, a hair smoothing composition disclosed herein comprises about 20 ppm to about 40 ppm hypochlorous acid or free available chlorine and about 0.5% to about 7% of a dimethicone, and about 0.5% to about 7% of a silicate clay. In still another aspect of this embodiment, a hair smoothing composition disclosed herein comprises about 20 ppm to about 35 ppm hypochlorous acid or free available chlorine and about 0.75% to about 6% of a dimethicone, and about 0.75% to about 6% of a silicate clay. In another aspect of this embodiment, a hair smoothing composition disclosed herein comprises about 25 ppm to about 30 ppm hypochlorous acid or free available chlorine and about 1% to about 5% of a dimethicone, and about 1% to about 5% of a silicate clay. In further aspects of this embodiment, a hair smoothing composition disclosed herein has a pH of about 8 to about 9.2, about 8.1 to about 9.1, about 8.2 to about 9.0, about 8.3 to about 8.9, about 8.4 to about 8.8, about 8.5 to about 8.7, or about 8.6.

In another aspect of this embodiment, a hair smoothing composition disclosed herein comprises about 25 ppm to about 30 ppm hypochlorous acid or free available chlorine, about 1.75% to about 2.75% of a dimethicone, and about 1.1% to about 2.1% of a silicate clay and has a pH of about 8.5 to about 8.7.

In an aspect of this embodiment, an anti-aging composition disclosed herein comprises about 18 ppm to about 30 ppm hypochlorous acid or free available chlorine, about 0.1% to about 5% of a dimethicone, a dimethicone/vinyl dimethicone crosspolymer, amodimethicone, or any combination thereof, and about 0.1% to about 7% of one or more rheology agents. In another aspect of this embodiment, an anti-aging composition disclosed herein comprises about 20 ppm to about 28 ppm hypochlorous acid or free available chlorine, about 0.15% to about 3% of a dimethicone, a dimethicone/vinyl dimethicone crosspolymer, amodimethicone, or any combination thereof, and about 0.25% to about 5% of one or more rheology agents. In yet another aspect of this embodiment, an anti-aging composition disclosed herein comprises about 21 ppm to about 27 ppm hypochlorous acid or free available chlorine, about 0.25% to about 2% of a dimethicone, a dimethicone/vinyl dimethicone crosspolymer, amodimethicone, or any combination thereof, and about 0.5% to about 4% of one or more rheology agents. In still another aspect of this embodiment, an anti-aging composition disclosed herein comprises about 22 ppm to about 26 ppm hypochlorous acid or free available chlorine, about 0.5% to about 1.5% of a dimethicone, a dimethicone/vinyl dimethicone crosspolymer, amodimethicone, or any combination thereof, and about 0.75% to about 3% of one or more rheology agents. In another aspect of this embodiment, an anti-aging composition disclosed herein comprises about 23 ppm to about 25 ppm hypochlorous acid or free available chlorine, about 0.75% to about 1.25% of a dimethicone, a dimethicone/vinyl dimethicone crosspolymer, amodimethicone, or any combination thereof, and about 1% to about 2% of one or more rheology agents. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In an aspect of this embodiment, an anti-aging composition disclosed herein comprises about 18 ppm to about 30 ppm hypochlorous acid or free available chlorine, about 0.1% to about 5% of a dimethicone, a dimethicone/vinyl dimethicone crosspolymer, amodimethicone, or any combination thereof, and about 0.1% to about 7% of a silicate clay. In another aspect of this embodiment, an anti-aging composition disclosed herein comprises about 20 ppm to about 28 ppm hypochlorous acid or free available chlorine, about 0.15% to about 3% of a dimethicone, a dimethicone/vinyl dimethicone crosspolymer, amodimethicone, or any combination thereof, and about 0.25% to about 5% of a silicate clay. In yet another aspect of this embodiment, an anti-aging composition disclosed herein comprises about 21 ppm to about 27 ppm hypochlorous acid or free available chlorine, about 0.25% to about 2% of a dimethicone, a dimethicone/vinyl dimethicone crosspolymer, amodimethicone, or any combination thereof, and about 0.5% to about 4% of a silicate clay. In still another aspect of this embodiment, an anti-aging composition disclosed herein comprises about 22 ppm to about 26 ppm hypochlorous acid or free available chlorine, about 0.5% to about 1.5% of a dimethicone, a dimethicone/vinyl dimethicone crosspolymer, amodimethicone, or any combination thereof, and about 0.75% to about 3% of a silicate clay. In another aspect of this embodiment, an anti-aging composition disclosed herein comprises about 23 ppm to about 25 ppm hypochlorous acid or free available chlorine, about 0.75% to about 1.25% of a dimethicone, a dimethicone/vinyl dimethicone crosspolymer, amodimethicone, or any combination thereof, and about 1% to about 2% of a silicate clay. In further aspects of this embodiment, an anti-aging composition disclosed herein has a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.

In another aspect of this embodiment, an anti-aging composition disclosed herein comprises about 23 ppm to about 25 ppm hypochlorous acid or free available chlorine, about 0.75% to about 1.25% of a dimethicone, a dimethicone/vinyl dimethicone crosspolymer, or any combination thereof, and about 1% to about 2% of a silicate clay and has a pH of about 7.1 to about 7.3.

In an aspect of this embodiment, an anti-aging composition disclosed herein comprises about 24 ppm to about 36 ppm hypochlorous acid or free available chlorine, about 0.1% to about 2.25% of a dimethicone/vinyl dimethicone crosspolymer, about 0.1% to about 2.25% of a dimethicone or amodimethicone, and about 0.5% to about 3.5% of a silicate clay. In another aspect of this embodiment, an anti-aging composition disclosed herein comprises about 26 ppm to about 34 ppm hypochlorous acid or free available chlorine, about 0.25% to about 2.0% of a dimethicone/vinyl dimethicone crosspolymer, about 0.25% to about 2.0% of a dimethicone or amodimethicone, and about 0.75% to about 3.25% of a silicate clay. In yet another aspect of this embodiment, an anti-aging composition disclosed herein comprises about 27 ppm to about 33 ppm hypochlorous acid or free available chlorine, about 0.5% to about 1.75% of a dimethicone/vinyl dimethicone crosspolymer, about 0.5% to about 1.75% of a dimethicone or amodimethicone, and about 1.0% to about 3.0% of a silicate clay. In still another aspect of this embodiment, an anti-aging composition disclosed herein comprises about 28 ppm to about 32 ppm hypochlorous acid or free available chlorine, about 0.75% to about 1.5% of a dimethicone/vinyl dimethicone crosspolymer, about 0.75% to about 1.5% of a dimethicone or amodimethicone, and about 1.25% to about 2.75% of a silicate clay. In another aspect of this embodiment, an anti-aging composition disclosed herein comprises about 29 ppm to about 31 ppm hypochlorous acid or free available chlorine, about 1.0% to about 1.25% of a dimethicone/vinyl dimethicone crosspolymer, about 1.0% to about 1.25% of a dimethicone or amodimethicone, and about 1.5% to about 2.5% of a silicate clay.

In further aspects of this embodiment, an anti-aging composition disclosed herein has a pH of about 8.1 to about 9.2, about 8.2 to about 9.1, about 8.3 to about 9.0, about 8.4 to about 8.9, about 8.5 to about 8.8, about 8.6 to about 8.8, or about 8.7. In an aspect of this embodiment, an anti-aging composition disclosed herein has a pH of about 8 to about 9, about 8.1 to about 9.0, about 8.2 to about 8.9, about 8.3 to about 8.8, about 8.4 to about 8.8, about 8.5 to about 8.7, or about 8.6.

In another aspect of this embodiment, an anti-aging composition disclosed herein comprises about 29 ppm to about 31 ppm hypochlorous acid or free available chlorine, about 1.0% to about 1.25% of a dimethicone/vinyl dimethicone crosspolymer, about 1.0% to about 1.25% of a dimethicone or amodimethicone, and about 1.5% to about 2.5% of a silicate clay and has a pH of about 8.5 to about 8.8.

In an aspect of this embodiment, an anti-aging composition disclosed herein comprises about 24 ppm to about 36 ppm hypochlorous acid or free available chlorine, about 0.01% to about 1.0% of a dimethicone/vinyl dimethicone crosspolymer, about 0.25% to about 1.75% of a dimethicone or amodimethicone, and about 0.5% to about 3.5% of a silicate clay. In another aspect of this embodiment, an anti-aging composition disclosed herein comprises about 26 ppm to about 34 ppm hypochlorous acid or free available chlorine, about 0.025% to about 0.75% of a dimethicone/vinyl dimethicone crosspolymer, about 0.5% to about 1.5% of a dimethicone or amodimethicone, and about 0.75% to about 3.25% of a silicate clay. In yet another aspect of this embodiment, an anti-aging composition disclosed herein comprises about 27 ppm to about 33 ppm hypochlorous acid or free available chlorine, about 0.05% to about 0.5% of a dimethicone/vinyl dimethicone crosspolymer, about 0.6% to about 1.2% of a dimethicone or amodimethicone, and about 1.0% to about 3.0% of a silicate clay. In still another aspect of this embodiment, an anti-aging composition disclosed herein comprises about 28 ppm to about 32 ppm hypochlorous acid or free available chlorine, about 0.1% to about 0.4% of a dimethicone/vinyl dimethicone crosspolymer, about 0.7% to about 1.1% of a dimethicone or amodimethicone, and about 1.25% to about 2.75% of a silicate clay. In another aspect of this embodiment, an anti-aging composition disclosed herein comprises about 29 ppm to about 31 ppm hypochlorous acid or free available chlorine, about 0.2% to about 0.3% of a dimethicone/vinyl dimethicone crosspolymer, about 0.8% to about 1.0% of a dimethicone or amodimethicone, and about 1.5% to about 2.5% of a silicate clay.

In further aspects of this embodiment, an anti-aging composition disclosed herein has a pH of about 8.1 to about 9.2, about 8.2 to about 9.1, about 8.3 to about 9.0, about 8.4 to about 8.9, about 8.5 to about 8.8, about 8.6 to about 8.8, or about 8.7. In an aspect of this embodiment, an anti-aging composition disclosed herein has a pH of about 8 to about 9, about 8.1 to about 9.0, about 8.2 to about 8.9, about 8.3 to about 8.8, about 8.4 to about 8.8, about 8.5 to about 8.7, or about 8.6.

In another aspect of this embodiment, an anti-aging composition disclosed herein comprises about 29 ppm to about 31 ppm hypochlorous acid or free available chlorine, about 0.2% to about 0.3% of a dimethicone/vinyl dimethicone crosspolymer, about 0.8% to about 1.0% of a dimethicone or amodimethicone, and about 1.5% to about 2.5% of a silicate clay and has a pH of about 8.6 to about 8.8.

In an aspect of this embodiment, a composition disclosed herein comprises about 75 ppm to about 125 ppm hypochlorous acid or free available chlorine, about 0.025% to about 1.0% of a dimethicone/vinyl dimethicone crosspolymer, about 3.0% to about 5.5% of a dimethicone or amodimethicone, and about 0.1% to about 2.0% of a silicate clay. In another aspect of this embodiment, a composition disclosed herein comprises about 80 ppm to about 120 ppm hypochlorous acid or free available chlorine, about 0.05% to about 0.75% of a dimethicone/vinyl dimethicone crosspolymer, about 3.25% to about 5.25% of a dimethicone or amodimethicone, and about 0.25% to about 1.5% of a silicate clay. In yet another aspect of this embodiment, a composition disclosed herein comprises about 85 ppm to about 115 ppm hypochlorous acid or free available chlorine, about 0.1% to about 0.5% of a dimethicone/vinyl dimethicone crosspolymer, about 3.5% to about 5.0% of a dimethicone or amodimethicone, and about 0.5% to about 1.25% of a silicate clay. In still another aspect of this embodiment, a composition disclosed herein comprises about 90 ppm to about 110 ppm hypochlorous acid or free available chlorine, about 0.125% to about 0.25% of a dimethicone/vinyl dimethicone crosspolymer, about 3.75% to about 4.75% of a dimethicone or amodimethicone, and about 0.6% to about 1.0% of a silicate clay. In another aspect of this embodiment, a composition disclosed herein comprises about 95 ppm to about 105 ppm hypochlorous acid or free available chlorine, about 0.15% to about 0.2% of a dimethicone/vinyl dimethicone crosspolymer, about 4.0% to about 4.5% of a dimethicone or amodimethicone, and about 0.7% to about 0.9% of a silicate clay.

In further aspects of this embodiment, a composition disclosed herein has a pH of about 6.5 to about 8.5, about 7.0 to about 8.0, about 7.1 to about 7.9, about 7.2 to about 7.8, about 7.3 to about 7.7, about 7.4 to about 7.6, or about 7.5.

In another aspect of this embodiment, a composition disclosed herein comprises about 95 ppm to about 105 ppm hypochlorous acid or free available chlorine, about 0.15% to about 0.2% of a dimethicone/vinyl dimethicone crosspolymer, about 4.0% to about 4.5% of a dimethicone or amodimethicone, and about 0.7% to about 0.9% of a silicate clay and has a pH of about 7.4 to about 7.6.

In an aspect of this embodiment, a composition disclosed herein comprises about 85 ppm to about 135 ppm hypochlorous acid or free available chlorine, about 0.25% to about 1.5% of a dimethicone/vinyl dimethicone crosspolymer, about 0.05% to about 0.75% of a dimethicone or amodimethicone, and about 1.0% to about 2.5% of a silicate clay. In another aspect of this embodiment, a composition disclosed herein comprises about 90 ppm to about 130 ppm hypochlorous acid or free available chlorine, about 0.5% to about 1.3% of a dimethicone/vinyl dimethicone crosspolymer, about 0.075% to about 0.5% of a dimethicone or amodimethicone, and about 1.2% to about 2.0% of a silicate clay. In yet another aspect of this embodiment, a composition disclosed herein comprises about 95 ppm to about 125 ppm hypochlorous acid or free available chlorine, about 0.6% to about 1.2% of a dimethicone/vinyl dimethicone crosspolymer, about 0.1% to about 0.4% of a dimethicone or amodimethicone, and about 1.3% to about 1.9% of a silicate clay. In still another aspect of this embodiment, a composition disclosed herein comprises about 100 ppm to about 120 ppm hypochlorous acid or free available chlorine, about 0.7% to about 1.1% of a dimethicone/vinyl dimethicone crosspolymer, about 0.15% to about 0.35% of a dimethicone or amodimethicone, and about 1.4% to about 1.8% of a silicate clay. In another aspect of this embodiment, a composition disclosed herein comprises about 105 ppm to about 115 ppm hypochlorous acid or free available chlorine, about 0.8% to about 1.0% of a dimethicone/vinyl dimethicone crosspolymer, about 0.2% to about 0.3% of a dimethicone or amodimethicone, and about 1.5% to about 1.7% of a silicate clay.

In further aspects of this embodiment, a composition disclosed herein has a pH of about 7.4 to about 8.6, about 7.5 to about 8.5, about 7.6 to about 8.4, about 7.7 to about 8.3, about 7.8 to about 8.2, about 7.9 to about 8.1, or about 8.0.

In another aspect of this embodiment, a composition disclosed herein comprises about 105 ppm to about 115 ppm hypochlorous acid or free available chlorine, about 0.8% to about 1.0% of a dimethicone/vinyl dimethicone crosspolymer, about 0.2% to about 0.3% of a dimethicone or amodimethicone, and about 1.5% to about 1.7% of a silicate clay and has a pH of about 7.9 to about 8.1.

In an aspect of this embodiment, a composition disclosed herein comprises about 160 ppm to about 210 ppm hypochlorous acid or free available chlorine, about 0.25% to about 1.5% of a dimethicone/vinyl dimethicone crosspolymer, about 0.05% to about 1.0% of a dimethicone or amodimethicone, and about 1.0% to about 2.5% of a silicate clay. In another aspect of this embodiment, a composition disclosed herein comprises about 165 ppm to about 205 ppm hypochlorous acid or free available chlorine, about 0.5% to about 1.3% of a dimethicone/vinyl dimethicone crosspolymer, about 0.1% to about 0.8% of a dimethicone or amodimethicone, and about 1.2% to about 2.0% of a silicate clay. In yet another aspect of this embodiment, a composition disclosed herein comprises about 170 ppm to about 200 ppm hypochlorous acid or free available chlorine, about 0.6% to about 1.2% of a dimethicone/vinyl dimethicone crosspolymer, about 0.2% to about 0.7% of a dimethicone or amodimethicone, and about 1.3% to about 1.9% of a silicate clay. In still another aspect of this embodiment, a composition disclosed herein comprises about 175 ppm to about 195 ppm hypochlorous acid or free available chlorine, about 0.7% to about 1.1% of a dimethicone/vinyl dimethicone crosspolymer, about 0.3% to about 0.6% of a dimethicone or amodimethicone, and about 1.4% to about 1.8% of a silicate clay. In another aspect of this embodiment, a composition disclosed herein comprises about 180 ppm to about 190 ppm hypochlorous acid or free available chlorine, about 0.8% to about 1.0% of a dimethicone/vinyl dimethicone crosspolymer, about 0.4% to about 0.5% of a dimethicone or amodimethicone, and about 1.5% to about 1.7% of a silicate clay.

In further aspects of this embodiment, a composition disclosed herein has a pH of about 7.3 to about 8.7, about 7.4 to about 8.6, about 7.5 to about 8.3, about 7.6 to about 8.2, about 7.7 to about 8.1, about 7.8 to about 8.0, or about 7.9.

In another aspect of this embodiment, a composition disclosed herein comprises about 180 ppm to about 190 ppm hypochlorous acid or free available chlorine, about 0.8% to about 1.0% of a dimethicone/vinyl dimethicone crosspolymer, about 0.4% to about 0.5% of a dimethicone or amodimethicone, and about 1.5% to about 1.7% of a silicate clay and has a pH of about 7.8 to about 8.0.

In an aspect of this embodiment, a composition disclosed herein comprises about 275 ppm to about 325 ppm hypochlorous acid or free available chlorine, about 0.25% to about 1.5% of a dimethicone/vinyl dimethicone crosspolymer, about 0.05% to about 1.0% of a dimethicone or amodimethicone, and about 1.0% to about 2.5% of a silicate clay. In another aspect of this embodiment, a composition disclosed herein comprises about 280 ppm to about 320 ppm hypochlorous acid or free available chlorine, about 0.5% to about 1.3% of a dimethicone/vinyl dimethicone crosspolymer, about 0.1% to about 0.8% of a dimethicone or amodimethicone, and about 1.2% to about 2.0% of a silicate clay. In yet another aspect of this embodiment, a composition disclosed herein comprises about 285 ppm to about 315 ppm hypochlorous acid or free available chlorine, about 0.6% to about 1.2% of a dimethicone/vinyl dimethicone crosspolymer, about 0.2% to about 0.7% of a dimethicone or amodimethicone, and about 1.3% to about 1.9% of a silicate clay. In still another aspect of this embodiment, a composition disclosed herein comprises about 290 ppm to about 310 ppm hypochlorous acid or free available chlorine, about 0.7% to about 1.1% of a dimethicone/vinyl dimethicone crosspolymer, about 0.3% to about 0.6% of a dimethicone or amodimethicone, and about 1.4% to about 1.8% of a silicate clay. In another aspect of this embodiment, a composition disclosed herein comprises about 295 ppm to about 305 ppm hypochlorous acid or free available chlorine, about 0.8% to about 1.0% of a dimethicone/vinyl dimethicone crosspolymer, about 0.4% to about 0.5% of a dimethicone or amodimethicone, and about 1.5% to about 1.7% of a silicate clay.

In further aspects of this embodiment, a composition disclosed herein has a pH of about 7.1 to about 8.3, about 7.2 to about 8.2, about 7.3 to about 8.1, about 7.4 to about 8.0, about 7.5 to about 7.9, about 7.6 to about 7.8, or about 7.7.

In another aspect of this embodiment, a composition disclosed herein comprises about 295 ppm to about 305 ppm hypochlorous acid or free available chlorine, about 0.8% to about 1.0% of a dimethicone/vinyl dimethicone crosspolymer, about 0.4% to about 0.5% of a dimethicone or amodimethicone, and about 1.5% to about 1.7% of a silicate clay and has a pH of about 7.6 to about 7.8.

In another embodiment, a hair smoothing composition disclosed herein comprises hypochlorous acid or free available chlorine, silicone and a rheology agent. In an aspect of this embodiment, a hair smoothing composition disclosed herein comprises about 23 ppm to about 35 ppm hypochlorous acid or free available chlorine, about 0.1% to about 10% of a silicone, and about 0.1% to about 7% of a rheology agent. In another aspect of this embodiment, a hair smoothing composition disclosed herein comprises about 25 ppm to about 33 ppm hypochlorous acid or free available chlorine, about 1% to about 8% of a silicone, and about 0.25% to about 5% of a rheology agent. In yet another aspect of this embodiment, a hair smoothing composition disclosed herein comprises about 26 ppm to about 32 ppm hypochlorous acid or free available chlorine, about 2% to about 7% of a silicone, and about 0.5% to about 4% of a rheology agent. In still another aspect of this embodiment, a hair smoothing composition disclosed herein comprises about 27 ppm to about 31 ppm hypochlorous acid or free available chlorine, about 3% to about 6% of a silicone, and about 0.75% to about 3% of a rheology agent. In another aspect of this embodiment, a hair smoothing composition disclosed herein comprises about 28 ppm to about 30 ppm hypochlorous acid or free available chlorine, about 4% to about 5% of a silicone, and about 1% to about 2% of a rheology agent. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

In an aspect of this embodiment, a hair smoothing composition disclosed herein comprises about 23 ppm to about 35 ppm hypochlorous acid or free available chlorine, about 0.1% to about 10% of one or more silicones, and about 0.1% to about 7% of one or more rheology agents. In another aspect of this embodiment, a hair smoothing composition disclosed herein comprises about 25 ppm to about 33 ppm hypochlorous acid or free available chlorine, about 1% to about 8% of one or more silicones, and about 0.25% to about 5% of one or more rheology agents. In yet another aspect of this embodiment, a hair smoothing composition disclosed herein comprises about 26 ppm to about 32 ppm hypochlorous acid or free available chlorine, about 2% to about 7% of one or more silicones, and about 0.5% to about 4% of one or more rheology agents. In still another aspect of this embodiment, a hair smoothing composition disclosed herein comprises about 27 ppm to about 31 ppm hypochlorous acid or free available chlorine, about 3% to about 6% of one or more silicones, and about 0.75% to about 3% of one or more rheology agents. In another aspect of this embodiment, a hair smoothing composition disclosed herein comprises about 28 ppm to about 30 ppm hypochlorous acid or free available chlorine, about 4% to about 5% of one or more silicones, and about 1% to about 2% of one or more rheology agents. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

In an aspect of this embodiment, a hair smoothing composition disclosed herein comprises about 23 ppm to about 35 ppm hypochlorous acid or free available chlorine, about 0.1% to about 10% of one or more silicone amines, one or more anionic silicones, or both one or more silicone amines and one or more anionic silicones, and about 0.1% to about 7% of one or more rheology agents. In another aspect of this embodiment, a hair smoothing composition disclosed herein comprises about 25 ppm to about 33 ppm hypochlorous acid or free available chlorine, about 1% to about 8% of one or more silicone amines, one or more anionic silicones, or both one or more silicone amines and one or more anionic silicones, and about 0.25% to about 5% of one or more rheology agents. In yet another aspect of this embodiment, a hair smoothing composition disclosed herein comprises about 26 ppm to about 32 ppm hypochlorous acid or free available chlorine, about 2% to about 7% of one or more silicone amines, one or more anionic silicones, or both one or more silicone amines and one or more anionic silicones, and about 0.5% to about 4% of one or more rheology agents. In still another aspect of this embodiment, a hair smoothing composition disclosed herein comprises about 27 ppm to about 31 ppm hypochlorous acid or free available chlorine, about 3% to about 6% of one or more silicone amines, one or more anionic silicones, or both one or more silicone amines and one or more anionic silicones, and about 0.75% to about 3% of one or more rheology agents. In another aspect of this embodiment, a hair smoothing composition disclosed herein comprises about 28 ppm to about 30 ppm hypochlorous acid or free available chlorine, about 4% to about 5% of one or more silicone amines, one or more anionic silicones, or both one or more silicone amines and one or more anionic silicones, and about 1% to about 2% of one or more rheology agents. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

In an aspect of this embodiment, a hair smoothing composition disclosed herein comprises about 23 ppm to about 35 ppm hypochlorous acid or free available chlorine, about 0.1% to about 10% of a dimethicone, a dimethicone/vinyl dimethicone crosspolymer, amodimethicone, or any combination thereof, and about 0.1% to about 7% of one or more rheology agents. In another aspect of this embodiment, a hair smoothing composition disclosed herein comprises about 25 ppm to about 33 ppm hypochlorous acid or free available chlorine, about 1% to about 8% of a dimethicone, a dimethicone/vinyl dimethicone crosspolymer, amodimethicone, or any combination thereof, and about 0.25% to about 5% of one or more rheology agents. In yet another aspect of this embodiment, a hair smoothing composition disclosed herein comprises about 26 ppm to about 32 ppm hypochlorous acid or free available chlorine, about 2% to about 7% of a dimethicone, a dimethicone/vinyl dimethicone crosspolymer, amodimethicone, or any combination thereof, and about 0.5% to about 4% of one or more rheology agents. In still another aspect of this embodiment, a hair smoothing composition disclosed herein comprises about 27 ppm to about 31 ppm hypochlorous acid or free available chlorine, about 3% to about 6% of a dimethicone, a dimethicone/vinyl dimethicone crosspolymer, amodimethicone, or any combination thereof, and about 0.75% to about 3% of one or more rheology agents. In another aspect of this embodiment, a hair smoothing composition disclosed herein comprises about 28 ppm to about 30 ppm hypochlorous acid or free available chlorine, about 4% to about 5% of a dimethicone, a dimethicone/vinyl dimethicone crosspolymer, amodimethicone, or any combination thereof, and about 1% to about 2% of one or more rheology agents. In further aspects of this embodiment, a hair smoothing composition disclosed herein has a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.

Aspects of the present specification disclose a kit. In one embodiment, the kit can comprise a container including an anti-aging composition disclosed herein, a container including a hair smoothing composition disclosed herein, or both. In another embodiment, a kit can comprise one or more containers containing each of the individual components separately or in combinations thereof. For example, a kit can comprise one container including the hypochlorous acid or free available chlorine and a second container including the and one or more silicones. The remainder of the components of an anti-aging composition, a hair smoothing composition disclosed herein may be included in either the first or second container, or may be separately including in at least a third container. Packaging of individual components into separate container can assist in prolonging the stability of the individual components, and thus shelf life of the product.

A kit disclosed herein can comprise a delivery or application system. The delivery or application system of the kit are useful for applying an anti-aging composition, a hair smoothing composition disclosed herein, and/or individual components disclosed herein to a site of interest, such as, e.g., at least one skin or hair region. A delivery or application system disclosed herein, includes, without limitation, one or more of an applicator brush, porous foam swab or pad, hollow tube, dipstick, or a combination thereof. In an embodiment, a kit comprises a single delivery or application system. In another embodiment, a kit comprises a plurality delivery or application systems. For example, in a 30-day supply kit, there can be 30 delivery or application systems, such that there is one delivery or application system per day for 30 days. Alternately, there can be 2, 10, 20, 30, 40, 50, 60, 90, 120, etc. delivery or application systems per kit. Within the kit, the delivery or application system may be packaged individually, or in sets of 2 or more. The delivery or application system can be packaged such that it remains sterile until use. In certain embodiments, a delivery or application system disclosed herein can be packaged in plastic sheaths. Further, to prevent contamination, delivery or application system disclosed herein is preferably single-use, disposable delivery or application system.

The kit can also comprise a set of instructions. The instructions may include information useful to the end user such as how to use a delivery or application system to apply an anti-aging composition, a hair smoothing composition, and/or individual components disclosed herein, and/or how often to apply an anti-aging composition, a hair smoothing composition, and/or individual components disclosed herein. In addition, such instructions may include information regarding how to mix the individual components disclosed herein to form an anti-aging composition or a hair smoothing composition disclosed herein. Such instructions can indicate that mixing should be done at a certain time before application, such as, e.g., just prior to use. Instructions disclosed herein may also include information regarding how to apply the individual components disclosed herein directly to a site of interest, such as, e.g., at least one skin or hair region, and in what order the individual components should be applied to such sites of interest.

The contents of the kit, including the container including a composition or component disclosed herein, the delivery or application system, and instructions, are enclosed in an outer casing. The outer casing can be a box, a sealed bag, a foil pouch, etc. In certain embodiments, the delivery system, container and instructions are enclosed in a box. In other embodiments of the kit, the container and instructions are contained in a first box, the delivery system is contained in a second box, and the first and second box are contained together in a third box.

Aspects of the present specification disclose a method to improve and/or support the appearance, beauty and/or health of a skin region in an individual. Other aspects of the present specification disclose a composition disclosed herein for use to improve and/or support the appearance, beauty and/or health of a skin region in an individual. Yet other aspects of the present specification disclose a use of a disclosed composition to improve and/or support the appearance, beauty and/or health of a skin region in an individual. Still other aspects of the present specification disclose a use of a disclosed composition in the manufacture of a medicament to improve and/or support the appearance, beauty and/or health of a skin region in an individual.

In one embodiment, a composition disclosed herein is applied topically to a skin region in an individual in order to improve and/or support the appearance, beauty and/or health of the skin region. In an aspect of this embodiment, an anti-aging composition disclosed herein is applied topically to a skin region in an individual in order to improve and/or support the appearance, beauty and/or health of the skin region. A skin region includes, without limitation, the face, forehead, lips, scalp, neck, shoulder, arms, hands, thighs, legs, knees, feet, chest, breast, back, groin, buttocks, and the like.

In one embodiment, one or more individual components disclosed herein are applied topically to a skin region in an individual in order to improve and/or support the appearance, beauty and/or health of the skin region. In an aspect of this embodiment, the one or more individual components include a first component including hypochlorous acid or free available chloride and a second component including one or more silicones. In another aspect, topical application of the one or more individual components occur in a specific order, such as, e.g., first applying a first component including hypochlorous acid or free available chloride and then applying a second component including one or more silicones. A skin region includes, without limitation, the face, forehead, lips, scalp, neck, shoulder, arms, hands, thighs, legs, knees, feet, chest, breast, back, groin, buttocks, and the like.

An improvement and/or support in the appearance, beauty and/or health of a skin region may be a restoration, maintenance and/or improvement of any attribute or characteristic of a skin region, including improvement of one or more signs of dermatological aging and/or environmental stress. Non-limiting examples of restoration, maintenance and/or improvement of any attribute or characteristic of a skin region include disappearance, reduction, and/or prevention of fine lines or wrinkles; disappearance, reduction, and/or prevention of skin sagging and/or skin sinking; disappearance, reduction, and/or prevention of skin thinning; disappearance, reduction, and/or prevention of skin dryness and/or itchiness; disappearance, reduction, and/or prevention of pigment spots and/or mottled skin; reduction of skin pore size; improvement in skin firmness; improvement in skin thickness, plumpness, and/or tautness; improvement in skin smoothness, suppleness and/or softness; improvement in skin tone, radiance, and/or clarity; improvement in procollagen, and/or collagen production; improvement in maintenance and remodeling of elastin; improvement in skin texture and/or promotion of retexturization; improvement in skin barrier repair and/or function; improvement in appearance of skin contours; restoration of skin luster and/or brightness; replenishment of essential nutrients and/or constituents in the skin; improvement of skin appearance decreased by aging and/or menopause; improvement in skin moisturization; increase in skin elasticity and/or resiliency; improvement of skin optical properties by light diffraction or reflection; or any combination thereof.

Aspects of the present specification disclose a method to improve and/or support the appearance, beauty and/or health of a hair region in an individual. Other aspects of the present specification disclose a composition disclosed herein for use to improve and/or support the appearance, beauty and/or health of a hair region in an individual. Yet other aspects of the present specification disclose a use of a disclosed composition to improve and/or support the appearance, beauty and/or health of a hair region in an individual. Still other aspects of the present specification disclose a use of a disclosed composition in the manufacture of a medicament to improve and/or support the appearance, beauty and/or health of a hair region in an individual.

In one embodiment, a composition disclosed herein is applied topically to a hair region in an individual in order to improve and/or support the appearance, beauty and/or health of the hair region. In an aspect of this embodiment, a hair smoothing composition disclosed herein is applied topically to a hair region in an individual in order to improve and/or support the appearance, beauty and/or health of the hair region. A hair region includes scalp hair, facial hair, eyebrows, body hair, and pubic hair.

In one embodiment, one or more individual components disclosed herein are applied topically to a hair region in an individual in order to improve and/or support the appearance, beauty and/or health of the hair region. In an aspect of this embodiment, the one or more individual components include a first component including hypochlorous acid or free available chloride and a second component including one or more silicones. In another aspect, topical application of the one or more individual components occur in a specific order, such as, e.g., first applying a first component including hypochlorous acid or free available chloride and then applying a second component including one or more silicones. A hair region includes scalp hair, facial hair, eyebrows, body hair, and pubic hair.

An improvement and/or support in the appearance, beauty and/or health of a hair region may be a restoration, maintenance and/or improvement of any attribute or characteristic of a hair region, including improvement of one or more signs of damage from environmental, physical or chemical exposure. Non-limiting examples of restoration, maintenance and/or improvement of any attribute or characteristic of a hair region include disappearance, reduction, and/or prevention of rough, dry and/or brittle hair fibers; disappearance, reduction, and/or prevention of hair fiber breakage; disappearance, reduction, and/or prevention of hair fiber frayed or split ends; disappearance, reduction, and/or prevention of undesirable hair color change; disappearance, reduction, and/or prevention of loss of hair shine; disappearance, reduction, and/or prevention of loss of hair; disappearance, reduction, and/or prevention of disentangled hair; improvement in hair suppleness; improvement in hair luster; improvement in hair shine; improvement in hair manageability; improvement in the combing or brushing of hair; or any combination thereof.

A method disclosed herein applies a composition or component disclosed herein to an individual. An individual refers to any mammal including a human, a horse, a cow, a sheep, a dog and a cat. As such, a method disclosed herein is for human use as well as veterinarian use.

A method disclosed herein is intended to beautifying, promoting attractiveness, cleanse and/or altering the appearance of skin and hair. In one embodiment, a method disclosed herein may be employed before the onset of any decline in any attribute or characteristic of skin and/or hair in order to promote and maintain the appearance, beauty and/or health of skin and/or hair. In another embodiment, a method disclosed herein may be employed to reverse and improve an attribute or characteristic of skin and/or hair that has declined in order to restore and/or maintain the appearance, beauty and/or health of skin and/or hair. In addition, a method disclosed herein may also provide a therapeutic or clinical effect in that application of a composition disclosed herein could reduce one or more symptoms associated with a skin or hair condition, such as, e.g., reduce itching, reduce pain, or promote healing of a skin abrasion.

A composition disclosed herein can be applied according to a method disclosed herein to a skin region and/or hair region. Application of a compositions disclosed herein can be by rubbing, pouring, sprinkling, or spraying on, or otherwise applied to the human body in accordance with the US FD&C Act, § 201(i). A composition disclosed herein can be applied by introducing the composition into or onto a solid support such as, e.g., a wipe, a patch, a strip, a towelette, a towel, a mitt, a glove, or a mask and then applying the composition to a skin or hair region. A composition disclosed herein can be applied by using a delivery device, such as, e.g., an aerosol dispenser, a dropper, a pump spray, a trigger spray or a squeeze bottle, to apply the composition to a skin or hair region.

A composition disclosed herein can be applied according to a method disclosed herein as often as needed and/or desired. A composition disclosed herein can be applied daily, every other day, every third of day, once a week, multiple times per week, once a month, multiple times per month, once a year or multiple times per year, as desired. A composition disclosed herein can be applied multiple times per day, e.g., twice a day, three times a day, four time a day, five times a day, six times a day or as often as desired.

A composition or method disclosed herein can be used with one or more additional skin care and/or hair care compositions as well as one or more additional other cosmetic compositions as part of a skin and/or hair care regimen. For example, in facial skin care, an individual typically use multiple products for cleansing, toning, and treating the skin of the face. As another example, in scalp hair care, an individual typically use multiple products for cleansing, conditioning and managing hair. Accordingly, the first product comprises a first composition typically capable of providing a first benefit to an individual, and the second product comprises a second composition typically capable of providing a second benefit to an individual. In the present specification, it should be understood that at least one of the compositions of the regime includes a composition disclosed herein, thereby improving and/or supporting the appearance, beauty and/or health of a skin region and/or a hair region in an individual. It should also be understood by one skilled in the art, that while the first product and second product can independently provide any benefit known in the art of the particular care regimen, in each particular multi-product care regimen, the first product and second product may include different compositions and thus, provide different benefits to an individual. Furthermore, as with the first product and second product of the care regimen, if more than two products are used in the multi-product care regimen, such as third, fourth, and/or fifth products (and more products if more than five products are desired), it should be recognized that the additional products should include active ingredients, each independently being capable of providing additional benefits to a user.

Aspects of the present specification can also be described as follows:
1. A composition comprising hypochlorous acid or free available chlorine and one or more silicones.
2. The composition according to embodiment 1, wherein the hypochlorous acid or free available chlorine is in an amount of 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition or at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition or at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition, or any combination thereof; or about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01% by weight of the composition
3. The composition according to embodiment 1 or 2, wherein the hypochlorous add or free available chlorine is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm, or any combination thereof; or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.
4. The composition according to any one of embodiments 1-3, wherein the one or more silicones comprise one or more non-volatile silicones, one or more volatile silicones, one or more silicone surfactants, or any combination thereof.
5. The composition according to embodiment 4, wherein the one or more non-volatile silicones include one or more polysiloxanes, one or more polysiloxane copolymers, one or more polyalkyl siloxanes, one or more polyaryl siloxanes, one or more polyalkylaryl siloxanes, one or more polyether siloxanes, one or more poly(alkylarylsiloxane)-(ethersiloxane) copolymers, one or more polydimethylsiloxanes, one or more polydiphenylsiloxanes, one or more poly(dimethylsiloxane)-(diphenylsiloxane) copolymers, one or more phenyl silicones, one or more polysilicate siloxane copolymers, or any combination thereof
6. The composition according to embodiment 4, wherein the one or more volatile silicones include one or more cyclomethicones.
7. The composition according to embodiment 4, wherein the one or more silicone surfactants include disodium PEG-12 dimethicone sulfosuccinate, PEG-12 dimethicone, PEG-10 dimethicone, PEG-8 dimethicone, PEG-7 dimethicone, PEG-7 dimethicone succinate, PEG-20/PPG-6 dimethicone, PEG-14/PPG-4 dimethicone, PEG-4/PPG-12 dimethicone, PEG-20/PPG-20 dimethicone, dimethylsiloxane and methyl(polyoxyethylene)siloxane copolymers, dimethylsiloxane and methyl(polyoxyethylene)siloxane and methyl(polyoxypropyl)siloxane copolymers, PEG-8 trisiloxane PEG-8/PPG-26 dimethicone, PEG-20/PPG-23 dimethicone, PEG-20/PPG-15 dimethicone, PEG-23/PPG-6 dimethicone, PEG-17 dimethicone, PPG-12 dimethicone, Quaternium-80, Quaternium 86, PEG-8 Distearmonium Chloride PG-Dimethicone, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-16/Glycidoxy Dimethicone Crosspolymer, Silicone Quaternium-17, Silicone Quaternium-18, Silicone Quaternium-19, Silicone Quaternium-20, Silicone Quaternium-21, Dimethicone Hydroxypropyl Trimonium Chloride, Hydroxyethyl Acetomonium PG-Dimethicone, Stearalkonium Dimethicone PEG-8 Phthalate, and Steardimonium Hydroxypropyl Panthenyl PEG-7 Dimethicone Phosphate Chloride.
8. The composition according to any one of embodiments 1-4, wherein the one or more silicones comprise one or more silicone amines, one or more anionic silicone, or both one or more silicone amines and one or more anionic silicone
9. The composition according to embodiment 8, wherein the one or more silicone amines includes an amodimethicone, a trimethylsilylamodimethicone, a trimethylsilylamodimethicone, a phenyltrimethicone, an aminopropyl phenyltrimethicone, a trimethyloxysilane, a triethyoxysilane, a tearoxytrimethylsilane, an aminopropyl triethyoxysilane, an alkyl modified amino silicone, an amino silane, a polyethylene glycol based amodimethicone, a bis-isobutyl PEG-14/amodimethicone copolymer, a bis-isobutyl PEG-15/amodimethicone copolymer, a bis-isobutyl PEG/PPG-20/35/amodimethicone copolymer, or any combination thereof.
10. The composition according to embodiment 9, wherein the polyethylene glycol based amodimethicone includes a PEG-7 amodimethicone or a PEG-8 amodimethicone.
11. The composition according to embodiment 9, wherein the modified aminosiloxane includes a Trideceth-9 PG-amodimethicone.
12. The composition according to any one of embodiments 8-11, wherein the one or more anionic silicones includes a polydimethylsiloxane, a silicone carboxylate, a silicone phosphate, a silicone sulfate, a silicone sulfosuccinate, a silicone sulfonate, a siloxane, or any combination thereof.
13. The composition according to embodiment 12, wherein the polydimethylsiloxane includes a dimethicone, a vinyl dimethicone, a dimethicone/vinyl dimethicone copolymer, a dimethicone/vinyl dimethicone crosspolymer or a cyclodimethicone.
14. The composition according to embodiment 13, wherein the cyclodimethicone includes hexamethylcyclotrisiloxane, octamethylcyclotetrasiloxane, decamethylcyclopentasiloxane and dodecamethylcyclohexasiloxane
15. The composition according to embodiment 12, wherein the silicone carboxylate includes a dimethicone PEG-7 phthalate or a dimethicone PEG-7 succinate.
16. The composition according to embodiment 12, wherein the silicone phosphate includes a dimethicone PEG-7 phosphate, a dimethicone PEG-8 phosphate, a dimethicone PEG-10 phosphate, a potassium dimethicone PEG-7 panthenyl phosphate, a dimethicone PEG/PPG-7/4 phosphate or a dimethicone PEG/PPG-12/4 phosphate.
17. The composition according to embodiment 12, wherein the siloxane includes a polysilicone-11, a polysilicone-15, a silica silylate, a silica dimethyl silylate, a trimethylsiloxysilicate, a trifluoropropyldimethyl/trimethylsiloxysilicate or an alkyldimethylsilyl polypropylsilsesquioxane.
18. The composition according to any one of embodiments 1-17, wherein the one or more silicones are an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, 10%, 15%, 20%, 25%, 30%, 35%, or 40% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, least 10%, least 15%, least 20%, least 25%, least 30%, least 35%, or least 40% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, most 10%, most 15%, most 20%, most 25%, most 30%, most 35%, or most 40% by weight of the composition, or any combination thereof; or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.
19. The composition according to any one of embodiments 8-18, wherein the one or more silicone amines and the one or more anionic silicones are in a ratio of from about 1:20 to about 20:1, about 1:15 to about 15:1, about 1:10 to about 10:1, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, or about 1:1.
20. The composition according to any one of embodiments 1-19, further comprising one or more rheology agents.
21. The composition according to embodiment 20, wherein the one or more rheology agents include a polyurethane-based polymer, a polyether polyol-based polymer, a castor oil derivative, a silica, a hydrophilic clay, a steareth-100/PEG-136/HDI copolymer, or any combination thereof.
22. The composition according to embodiment 21, wherein the polyurethane-based polymer includes bis-lauryl cocaminopropylamine/HDI/PEG-100 copolymer (and) butylene glycol or bis-C₁₆₋₂₀ isoalkoxy TMHDI/PEG-90 copolymer.
23. The composition according to embodiment 21, wherein the polyether polyol-based polymer includes PEG/PPG-450/50 trimethylolpropane dodecyl ether.
24. The composition according to embodiment 21, wherein the castor oil derivative includes trihydroxystearin.
25. The composition according to embodiment 21, wherein the hydrophilic clay includes a hectorite clay or a silicate clay.
26. The composition according to any one of embodiments 20-25, wherein the rheology agent is in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, or 10% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition, or any combination thereof; or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.
27. The composition according to any one of embodiments 1-26, further comprising one or more surfactants.
28. The composition according to embodiment 26, wherein the one or more surfactants include one or more ionic surfactants, one or more zwitterionic (amphoteric) surfactants, one or more non-ionic surfactants, one or more biosurfactants, or any combination thereof
29. The composition according to embodiment 28, wherein the one or more ionic surfactants include one or more anionic surfactants, one or more cationic surfactants, or any combination thereof.
30. The composition according to embodiment 29, wherein the one or more anionic surfactants include one or more alkyl sulfates, one or more alkyl ether sulfates, one or more docusates like dioctyl sodium sulfosuccinates, one or more sulfonate fluorosurfactants, one or more alkyldiphenyloxide, one or more alkyldiphenyloxide disulfonates, one or more potassium phosphate polyether esters, one or more alkyl benzene sulfonates, one or more alkyl aryl ether phosphates, one or more alkyl ether phosphates, one or more alkyl carboxylates, one or more carboxylate fluorosurfactants, sodium lauroyl sarcosinate, sodium hexyldiphenyl ether sulfonate (DOWFAX™ C6L), or any combination thereof.
31. The composition according to embodiment 30, wherein the one or more alkyl sulfates include ammonium lauryl sulfate, sodium lauryl sulfate (SDS), or any combination thereof.
32. The composition according to embodiment 30, wherein the one or more alkyl ether sulfates include sodium laureth sulfate, sodium myreth sulfate, or any combination thereof.
33. The composition according to embodiment 30, wherein the one or more docusates include dioctyl sodium sulfosuccinate.
34. The composition according to embodiment 30, wherein the one or more sulfonate fluorosurfactants include perfluorooctanesulfonate (PFOS), perfluorobutanesulfonate, or any combination thereof.
35. The composition according to embodiment 30, wherein the one or more alkyldiphenyloxide disulfonates include DOWFAX™ 2A1 (Disodium Lauryl Phenyl Ether Disulfonate), DOWFAX™ 3B2 (Disodium Decyl Phenyl Ether Disulfonate), DOWFAX™ C10L (Disodium Decyl Phenyl Ether Disulfonate), DOWFAX™ 2EP, DOWFAX™ 8390 (Disodium Cetyl Phenyl Ether Disulfonate), or any combination thereof.
36. The composition according to embodiment 30, wherein the one or more potassium phosphate polyether esters include TRITON™ H-55, TRITON™ H-66, or any combination thereof.
37. The composition according to embodiment 30, wherein the one or more alkyl carboxylates include fatty acid salts, sodium stearate, or any combination thereof.
38. The composition according to embodiment 30, wherein the one or more carboxylate fluorosurfactants include perfluorononanoate, perfluorooctanoate, or any combination thereof.
39. The composition according to embodiment 29, wherein the one or more cationic surfactants include one or more alkyltrimethylammonium salts, cetylpyridinium chloride (CPC), polyethoxylated tallow amine (POEA), benzalkonium chloride (BAC), benzethonium chloride (BZT), 5-Bromo-5-nitro-1,3-dioxane, dimethyldioctadecylammonium chloride, dioctadecyldimethylammonium bromide (DODAB), octenidine dihydrochloride, or any combination thereof.
40. The composition according to embodiment 39, wherein the one or more alkyltrimethylammonium salts include cetyl trimethylammonium bromide (CTAB), cetyl trimethylammonium chloride (CTAC), or any combination thereof.
41. The composition according to embodiment 28, wherein the one or more Zwitterionic surfactants include one or more sultaines one or more betaines, one or more lecithins, 3-[(3-Cholamidopropyl)dimethylammonio]-1-propanesulfonate (CHAPS), or any combination thereof.
42. The composition according to embodiment 41, wherein the one or more sultaines include cocamidopropyl hydroxysultaine.
43. The composition according to embodiment 41, wherein the one or more betaines include cocamidopropyl betaine.
44. The composition according to embodiment 28, wherein the one or more non-ionic surfactants include one or more polyether nonionic surfactants, one or more polyhydroxyl nonionic surfactants, or any combination thereof.
45. The composition according to embodiment 28, wherein the one or more non-ionic surfactants include one or more alcohol ethoxylates, one or more alkylphenol ethoxylates, one or more phenol ethoxylates, one or more amide ethoxylates, one or more glyceride ethoxylates, one or more fatty acid ethoxylates, one or more fatty amine ethoxylates, or any combination thereof.
46. The composition according to embodiment 28, wherein the one or more non-ionic surfactants include one or more polyoxyethylene glycol sorbitan alkyl esters (or ethoxylated sorbital esters), one or more sorbital esters, one or more polyglycerol esters, one or more ethoxylated polyglycerol esters, one or more alkyl glucosides, one or more ethoxylated alkyl glucosides, one or more sucrose esters, one or more ethoxylated sucrose ester, one or more amine oxides, one or more ethoxylated alcohols, one or more ethoxylated aliphatic alcohols, one or more alkylamines, one or more ethoxylated alkylamines, one or more ethoxylated alkyl phenols, one or more alkyl polysaccharides, one or more ethoxylated alkyl polysaccharides, one or more ethoxylated fatty acids, one or more ethoxylated fatty alcohols, one or more ethoxylated fatty amines, one or more poloxamers (polyethylene-polypropylene copolymers), one or more linear secondary alcohol ethoxylates, one or more alkyl phenol polyglycol ethers, one or more polyethylene glycol alkyl aryl ethers, one or more polyoxyethylene glycol alkyl ethers, one or more polyoxyethylene glycol octylphenol ethers, one or more polyoxyethylene glycol alkylphenol ethers, one or more phenoxypolyethoxylethanols, one or more glucoside alkyl ethers, one or more maltoside alkyl ethers, one or more thioglucoside alkyl ethers, one or more digitonins, one or more glycerol alkyl esters, one or more alkyl aryl polyether sulfates, one or more alcohol sulfonates, one or more sorbitan alkyl esters, one or more glycerol alkyl esters, one or more cocamide ethanolamines, sucrose monolaurate, dodecyl dimethylamine oxide, sodium cholate, 2-dodecoxyethanol (LUBROL®-PX), or any combination thereof.
47. The composition according to embodiment 46, wherein the one or more polyoxyethylene glycol sorbitan alkyl esters (or ethoxylated sorbital esters) include polysorbate 20 sorbitan monooleate (TWEEN® 20), polysorbate 40 sorbitan monooleate (TWEEN® 40), polysorbate 60 sorbitan monooleate (TWEEN® 60), polysorbate 61 sorbitan monooleate (TWEEN® 61), polysorbate 65 sorbitan monooleate (TWEEN® 65), polysorbate 80 sorbitan monooleate (TWEEN® 80), polysorbate 81 sorbitan monooleate (TWEEN® 81), polysorbate 85 sorbitan monooleate (TWEEN® 85), or any combination thereof.
48. The composition according to embodiment 46, wherein the one or more sorbital esters include sorbitan monooleate, sorbitan monolaurate, sorbitan monopalmitate, sorbitan monostearate, sorbitan tristearate, or any combination thereof.
49. The composition according to embodiment 46, wherein the one or more polyglycerol esters include glycerol monooleate, glycerol monolaurate, glycerol monopalmitate, glycerol monostearate, glycerol trioleate, glycerol ricinoleate, glycerol tristearate, or any combination thereof.
50. The composition according to embodiment 46, wherein the one or more alkyl glucosides include arachidyl glucoside, C₁₂₋₂₀ alkyl glucoside, caprylyl/capryl glucoside, cetearyl glucoside, coco-glucoside, ethyl glucoside and lauryl glucoside, decyl glucoside, or any combination thereof.
51. The composition according to embodiment 46, wherein the one or more sucrose esters include sucrose monooleate, sucrose monolaurate, sucrose monopalmitate, sucrose monostearate, sucrose trioleate, sucrose ricinoleate, sucrose tristearate, sucrose diglycerides, sucrose triacetate, or any combination thereof.
52. The composition according to embodiment 46, wherein the one or more ethoxylated alkyl phenols include ethoxylated nonyl phenol, ethoxylated octyl phenol, or any combination thereof.
53. The composition according to embodiment 46, wherein the one or more ethoxylated fatty acids include ethoxylated castor oil.
54. The composition according to embodiment 46, wherein the one or more ethoxylated fatty alcohols include ethoxylated ceto-oleyl alcohol, ethoxylated ceto-stearyl alcohol, ethoxylated decyl alcohol, ethoxylated dodecyl alcohol, ethoxylated tridecyl alcohol, or any combination thereof.
55. The composition according to embodiment 46, wherein the one or more poloxamers (polyethylene-polypropylene copolymers) include Poloxamer 124 (PLURONIC® L44), Poloxamer 181 (PLURONIC® L61), Poloxamer 182 (PLURONIC® L62), Poloxamer 184 (PLURONIC® L64), Poloxamer 188 (PLURONIC®F68), Poloxamer 237 (PLURONIC®F87), Poloxamer 338 (PLURONIC®L108), Poloxamer 407 (PLURONIC® F127), or any combination thereof.
56. The composition according to embodiment 46, wherein the one or more linear secondary alcohol ethoxylates include TERGITOL™ 15-S-5, TERGITOL™ 15-S-7, TERGITOL™ 15-S-9, TERGITOL™ 15-S-12, TERGITOL™ 15-S-15, TERGITOL™ 15-S-20, TERGITOL™ 15-S-30, TERGITOL™ 15-S-40, or any combination thereof.
57. The composition according to embodiment 46, wherein the one or more polyoxyethylene glycol alkyl ethers include octaethylene glycol monododecyl ether, pentaethylene glycol monododecyl ether, BRIJ® 30, BRIJ® 35, or any combination thereof.
58. The composition according to embodiment 46, wherein the one or more polyoxyethylene glycol octylphenol ethers include polyoxyethylene (4-5) p-t-octyl phenol (TRITON® X-45), polyoxyethylene octyl phenyl ether (TRITON® X-100), or any combination thereof.
59. The composition according to embodiment 46, wherein the one or more polyoxyethylene glycol alkylphenol ethers include Nonoxynol-9.
60. The composition according to embodiment 46, wherein the one or more phenoxypolyethoxylethanols include nonylphenoxypolyethoxylethanol, octylphenoxypolyethoxylethanol (IGEPAL® CA-630 or NONIDET™ P-40), or any combination thereof.
61. The composition according to embodiment 46, wherein the one or more glucoside alkyl ethers include octyl glucopyranoside.
62. The composition according to embodiment 46, wherein the one or more maltoside alkyl ethers include dodecyl maltopyranoside.
63. The composition according to embodiment 46, wherein the one or more thioglucoside alkyl ethers include heptyl thioglucopyranoside.
64. The composition according to embodiment 46, wherein the one or more glycerol alkyl esters include glyceryl laurate.
65. The composition according to embodiment 56, wherein the one or more cocamide ethanolamines include cocamide monoethanolamine, cocamide diethanolamine, or any combination thereof.
66. The composition according to embodiment 28, wherein the one or more biosurfactants include one or more bio-based anionic surfactants, one or more bio-based nonionic surfactants, or any combination thereof.
67. The composition according to embodiment 66, wherein the one or more bio-based anionic surfactants include STEPONOL® AM 30-KE, an ammonium lauryl sulfate, and STEPONOL® EHS, a sodium 2-ethyl hexyl sulfate, or any combination thereof.
68. The composition according to embodiment 66, wherein the one or more bio-based nonionic surfactants include STEPOSOL® MET-10U, a metathesis-derived, nonionic surfactant that is an unsaturated, short chain amide.
69. The composition according to any one of embodiments 27-68, wherein the one or more surfactants are in an amount of about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0% or about 10.0% by weight of the composition, or at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, or at least 10.0% by weight of the composition, or at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0% or at most 10.0% by weight of the composition, or about 0.1% to about 0.5%, about 0.1% to about 0.75%, about 0.1% to about 1.0%, about 0.1% to about 1.5%, about 0.1% to about 2.0%, about 0.1% to about 2.5%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0% or about 5.0% to about 15.0% by weight of the composition.
70. The composition according to any one of embodiments 1-69, further comprising one or more carriers.
71. The composition according to embodiment 70, wherein the one or more carrier includes an aqueous carrier, a semi-solid carrier, a solid carrier, or any combination thereof.
72. The composition according to embodiment 70 or 71, wherein the one or more carrier includes water, a vegetable oil, a mineral oil, an ester oil, an ether, an alcohol, a fatty alcohol, an isoparaffin, a hydrocarbon oil, a polyol, a wax, or any combination thereof.
73. The composition according to embodiment 72, wherein the ester oil includes octal palmitate, isopropyl myristate or isopropyl palmitate.
74. The composition according to embodiment 72, wherein the ether includes dicapryl ether or dimethyl isosorbide.
75. The composition according to embodiment 72, wherein the alcohol includes ethanol or isopropanol.
76. The composition according to embodiment 72, wherein the fatty alcohol includes cetyl alcohol, cetearyl alcohol, stearyl alcohol or behenyl alcohol.
77. The composition according to embodiment 72, wherein the isoparaffin includes isooctane, isododecane (IDD) or isohexadecane.
78. The composition according to embodiment 72, wherein the hydrocarbon oil includes mineral oil, petrolatum, isoeicosane or a polyolefin.
79. The composition according to embodiment 72, wherein the polyol includes propylene glycol, glycerin, butylene glycol, pentylene glycol, hexylene glycol, or caprylyl glycol.
80. The composition according to embodiment 72, wherein the wax includes beeswax, carnauba, ozokerite, microcrystalline wax, polyethylene wax, or a botanical wax.
81. The composition according to any one of embodiments 70-80, wherein the one or more carriers are in an amount of at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% by weight of the composition or at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98% or at most 99% by weight of the composition or about 25% to about 50%, about 25% to about 75%, about 25% to about 90%, about 25% to about 95%, about 25% to about 96%, about 25% to about 97%, about 25% to about 98%, about 25% to about 99%, about 50% to about 75%, about 50% to about 90%, about 50% to about 95%, about 50% to about 96%, about 50% to about 97%, about 50% to about 98%, about 50% to about 99%, about 75% to about 80%, about 75% to about 85%, about 75% to about 90%, about 75% to about 95%, about 75% to about 96%, about 75% to about 97%, about 75% to about 98%, about 75% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, or about 95% to about 99%, by weight of the composition.
82. The composition according to any one of embodiments 1-81, further comprising one or more additional ingredients.
83. The composition according to embodiment 82, wherein the one or more additional ingredient includes a humectant, a preservative, an emulsifier, a fragrance, a chelating agent, an astringent, a shine control agent, a colorant agent, a soothing agent, a calming agent, or any combination thereof.
84. The composition according to any one of embodiments 1-83, having a pH of about 2 to about 5, about 2 to about 5.5, about 2 to about 6, about 2 to about 6.5, about 2 to about 7, about 2 to about 7.5, about 2 to about 8, about 2 to about 8.5, about 2 to about 9, about 2.5 to about 5, about 2.5 to about 5.5, about 2.5 to about 6, about 2.5 to about 6.5, about 2.5 to about 7, about 2.5 to about 7.5, about 2.5 to about 8, about 2.5 to about 8.5, about 2.5 to about 9, about 3 to about 5, about 3 to about 5.5, about 3 to about 6, about 3 to about 6.5, about 3 to about 7, about 3 to about 7.5, about 3 to about 8, about 3 to about 8.5, about 3 to about 9, about 3.5 to about 5, about 3.5 to about 5.5, about 3.5 to about 6, about 3.5 to about 6.5, about 3.5 to about 7, about 3.5 to about 7.5, about 3.5 to about 8, about 3.5 to about 8.5, about 3.5 to about 9, about 4 to about 5, about 4 to about 5.5, about 4 to about 6, about 4 to about 6.5, about 4 to about 7, about 4 to about 7.5, about 4 to about 8, about 4 to about 8.5, about 4 to about 9, about 4.5 to about 5, about 4.5 to about 5.5, about 4.5 to about 6, about 4.5 to about 6.5, about 4.5 to about 7, about 4.5 to about 7.5, about 4.5 to about 8, about 4.5 to about 8.5, about 4.5 to about 9, about 5 to about 5.5, about 5 to about 6, about 5 to about 6.5, about 5 to about 7, about 5 to about 7.5, about 5 to about 8, about 5 to about 8.5, about 5 to about 9, about 5.5 to about 6, about 5.5 to about 6.5, about 5.5 to about 7, about 5.5 to about 7.5, about 5.5 to about 8, about 5.5 to about 8.5, about 5.5 to about 9, about 6 to about 6.5, about 6 to about 7, about 6 to about 7.5, about 6 to about 8, about 6 to about 8.5, about 6 to about 9, about 6.5 to about 7, about 6.5 to about 7.5, about 6.5 to about 8, about 6.5 to about 8.5, about 6.5 to about 9, about 7 to about 7.5, about 7 to about 8, about 7 to about 8.5, about 7 to about 9, about 7.5 to about 8, about 7.5 to about 8.5, about 7.5 to about 9, about 8 to about 8.5, about 8 to about 9, or about 8.5 to about 9.
85. The composition according to any one of embodiments 1-83, having a pH of about 6 to about 8, about 6.7 to about 7.7, about 6.8 to about 7.6, about 6.9 to about 7.5, about 7.0 to about 7.4, about 7.1 to about 7.3, or about 7.2. In an aspect of this embodiment, an anti-aging composition disclosed herein is between, e.g., about 6 to about 8, about 6.7 to about 7.7, about 6.8 to about 7.6, about 6.9 to about 7.5, about 7.0 to about 7.4, about 7.1 to about 7.3, or about 7.2.
86. The composition according to any one of embodiments 1-83, having a pH of about 5.0 to about 6.5, about 5.2 to about 6.2, about 5.3 to about 6.1, about 5.4 to about 6.0, about 5.5 to about 5.9, about 5.6 to about 5.8, or about 5.7. In an aspect of this embodiment, a hair smoothing composition disclosed herein is between, e.g., about 5.0 to about 6.5, about 5.2 to about 6.2, about 5.3 to about 6.1, about 5.4 to about 6.0, about 5.5 to about 5.9, about 5.6 to about 5.8, or about 5.7.
87. The composition according to any one of embodiments 1-86, having a stability of about 1 day, about 2 days, 3 days, 4 days, 5 days, 6, days, 7 days, 8 days, 9 days, 10 days, 11 days, 12 days, 13 days, 14 days or 15 days or at least 1 day, at least 2 days, at least 3 days, at least 4 days, at least 5 days, at least 6, days, at least 7 days, at least 8 days, at least 9 days, at least 10 days, at least 11 days, at least 12 days, at least 13 days, at least 14 days or at least 15 days or at most 1 day, at most 2 days, at most 3 days, at most 4 days, at most 5 days, at most 6, days, at most 7 days, at most 8 days, at most 9 days, at most 10 days, at most 11 days, at most 12 days, at most 13 days, at most 14 days or at most 15 days or about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 1 day to about 13 days, about 1 day to about 14 days, about 1 day to about 15 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 2 days to about 13 days, about 2 days to about 14 days, about 2 days to about 15 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 3 days to about 13 days, about 3 days to about 14 days, about 3 days to about 15 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 4 days to about 13 days, about 4 days to about 14 days, about 4 days to about 15 days, about 5 days to about 6 days, about 5 days to about 7 days, about 5 days to about 8 days, about 5 days to about 9 days, about 5 days to about 10 days, about 5 days to about 11 days, about 5 days to about 12 days, about 5 days to about 13 days, about 5 days to about 14 days, about 5 days to about 15 days, about 6 days to about 7 days, about 6 days to about 8 days, about 6 days to about 9 days, about 6 days to about 10 days, about 6 days to about 11 days, about 6 days to about 12 days, about 6 days to about 13 days, about 6 days to about 14 days, about 6 days to about 15 days, about 7 days to about 8 days, about 7 days to about 9 days, about 7 days to about 10 days, about 7 days to about 11 days, about 7 days to about 12 days, about 7 days to about 13 days, about 7 days to about 14 days, about 7 days to about 15 days, about 8 days to about 9 days, about 8 days to about 10 days, about 8 days to about 11 days, about 8 days to about 12 days, about 8 days to about 13 days, about 8 days to about 14 days, about 8 days to about 15 days, about 9 days to about 10 days, about 9 days to about 11 days, about 9 days to about 12 days, about 9 days to about 13 days, about 9 days to about 14 days, about 9 days to about 15 days, about 10 days to about 11 days, about 10 days to about 12 days, about 10 days to about 13 days, about 10 days to about 14 days, about 10 days to about 15 days, about 11 days to about 12 days, about 11 days to about 13 days, about 11 days to about 14 days, about 11 days to about 15 days, about 12 days to about 13 days, about 12 days to about 14 days, about 12 days to about 15 days, about 13 days to about 14 days, about 13 days to about 15 days, or about 14 days to about 15 days.
88. The composition according to any one of embodiments 1-86, having a stability of about 1 week, about 2 weeks, 3 weeks, 4 weeks, 5 weeks, 6, weeks, 7 weeks, 8 weeks, 9 weeks, 10 weeks, 11 weeks, 12 weeks, 13 weeks, 14 weeks or 15 weeks or at least 1 week, at least 2 weeks, at least 3 weeks, at least 4 weeks, at least 5 weeks, at least 6, weeks, at least 7 weeks, at least 8 weeks, at least 9 weeks, at least 10 weeks, at least 11 weeks, at least 12 weeks, at least 13 weeks, at least 14 weeks or at least 15 weeks or at most 1 week, at most 2 weeks, at most 3 weeks, at most 4 weeks, at most 5 weeks, at most 6, weeks, at most 7 weeks, at most 8 weeks, at most 9 weeks, at most 10 weeks, at most 11 weeks, at most 12 weeks, at most 13 weeks, at most 14 weeks or at most 15 weeks or about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 1 week to about 13 weeks, about 1 week to about 14 weeks, about 1 week to about 15 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 2 weeks to about 13 weeks, about 2 weeks to about 14 weeks, about 2 weeks to about 15 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 3 weeks to about 13 weeks, about 3 weeks to about 14 weeks, about 3 weeks to about 15 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 4 weeks to about 13 weeks, about 4 weeks to about 14 weeks, about 4 weeks to about 15 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 5 weeks to about 13 weeks, about 5 weeks to about 14 weeks, about 5 weeks to about 15 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 6 weeks to about 13 weeks, about 6 weeks to about 14 weeks, about 6 weeks to about 15 weeks, about 7 weeks to about 8 weeks, about 7 weeks to about 9 weeks, about 7 weeks to about 10 weeks, about 7 weeks to about 11 weeks, about 7 weeks to about 12 weeks, about 7 weeks to about 13 weeks, about 7 weeks to about 14 weeks, about 7 weeks to about 15 weeks, about 8 weeks to about 9 weeks, about 8 weeks to about 10 weeks, about 8 weeks to about 11 weeks, about 8 weeks to about 12 weeks, about 8 weeks to about 13 weeks, about 8 weeks to about 14 weeks, about 8 weeks to about 15 weeks, about 9 weeks to about 10 weeks, about 9 weeks to about 11 weeks, about 9 weeks to about 12 weeks, about 9 weeks to about 13 weeks, about 9 weeks to about 14 weeks, about 9 weeks to about 15 weeks, about 10 weeks to about 11 weeks, about 10 weeks to about 12 weeks, about 10 weeks to about 13 weeks, about 10 weeks to about 14 weeks, about 10 weeks to about 15 weeks, about 11 weeks to about 12 weeks, about 11 weeks to about 13 weeks, about 11 weeks to about 14 weeks, about 11 weeks to about 15 weeks, about 12 weeks to about 13 weeks, about 12 weeks to about 14 weeks, about 12 weeks to about 15 weeks, about 13 weeks to about 14 weeks, about 13 weeks to about 15 weeks, or about 14 weeks to about 15 weeks.
89. The composition according to any one of embodiments 1-86, having a stability of about 1 month, about 2 months, 3 months, 4 months, 5 months, 6, months, 7 months, 8 months, 9 months, 10 months, 11 months, 12 months, 13 months, 14 months or 15 months or at least 1 month, at least 2 months, at least 3 months, at least 4 months, at least 5 months, at least 6, months, at least 7 months, at least 8 months, at least 9 months, at least 10 months, at least 11 months, at least 12 months, at least 13 months, at least 14 months or at least 15 months or at most 1 month, at most 2 months, at most 3 months, at most 4 months, at most 5 months, at most 6, months, at most 7 months, at most 8 months, at most 9 months, at most 10 months, at most 11 months, at most 12 months, at most 13 months, at most 14 months or at most 15 months or about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 1 month to about 13 months, about 1 month to about 14 months, about 1 month to about 15 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 2 months to about 13 months, about 2 months to about 14 months, about 2 months to about 15 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 3 months to about 13 months, about 3 months to about 14 months, about 3 months to about 15 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 4 months to about 13 months, about 4 months to about 14 months, about 4 months to about 15 months, about 5 months to about 6 months, about 5 months to about 7 months, about 5 months to about 8 months, about 5 months to about 9 months, about 5 months to about 10 months, about 5 months to about 11 months, about 5 months to about 12 months, about 5 months to about 13 months, about 5 months to about 14 months, about 5 months to about 15 months, about 6 months to about 7 months, about 6 months to about 8 months, about 6 months to about 9 months, about 6 months to about 10 months, about 6 months to about 11 months, about 6 months to about 12 months, about 6 months to about 13 months, about 6 months to about 14 months, about 6 months to about 15 months, about 7 months to about 8 months, about 7 months to about 9 months, about 7 months to about 10 months, about 7 months to about 11 months, about 7 months to about 12 months, about 7 months to about 13 months, about 7 months to about 14 months, about 7 months to about 15 months, about 8 months to about 9 months, about 8 months to about 10 months, about 8 months to about 11 months, about 8 months to about 12 months, about 8 months to about 13 months, about 8 months to about 14 months, about 8 months to about 15 months, about 9 months to about 10 months, about 9 months to about 11 months, about 9 months to about 12 months, about 9 months to about 13 months, about 9 months to about 14 months, about 9 months to about 15 months, about 10 months to about 11 months, about 10 months to about 12 months, about 10 months to about 13 months, about 10 months to about 14 months, about 10 months to about 15 months, about 11 months to about 12 months, about 11 months to about 13 months, about 11 months to about 14 months, about 11 months to about 15 months, about 12 months to about 13 months, about 12 months to about 14 months, about 12 months to about 15 months, about 13 months to about 14 months, about 13 months to about 15 months, or about 14 months to about 15 months.
90. The composition according to any one of embodiments 1-86, having a stability of about 1 year, about 2 years, about 3 years, about 4 years, or about 5 years or at least 1 year, at least 2 years, at least 3 years, at least 4 years, or at least 5 years or at most 1 year, at most 2 years, at most 3 years, at most 4 years, or at most 5 years or about 1 year to about 2 years, about 1 year to about 3 years, about 1 year to about 4 years, about 1 year to about 5 years, about 2 years to about 3 years, about 2 years to about 4 years, about 2 years to about 5 years, about 3 years to about 4 years, about 3 years to about 5 years, or about 4 years to about 5 years.
91. The composition according to any one of embodiments 1-90, formulated as a single-phase formulation, a biphasic formulation, or a multiphasic formulation.
92. The composition according to any one of embodiments 1-90, formulated as a liquid composition, a colloidal composition, a semi-solid composition, or a solid composition.
93. The composition according to any one of embodiments 1-90, formulated as a liquid aerosol, a foam, an emulsion, a gel, a sol, or a solid sol.
94. The composition according to any one of embodiments 1-90, formulated as a spray, a liquid aerosol, a wash, an aftershave, a perfume, a body mist, a lotion, a cream, a salve, a waxing composition, a mousse, a shampoo, a conditioner, an ointment, a soap, a toner, a foundation, a stick, a film forming product, a suspension, or an emollient.
95. A method to improve and/or support the appearance, beauty and/or health of a skin region in an individual, the method comprising topically applying a composition as defined in any one of embodiment 1-94, wherein application of the composition restores, maintains and/or improves an attribute or a characteristic of the skin region, thereby improving and/or supporting the appearance, beauty and/or health of the skin region.
96. The method according to embodiment 95, wherein the skin region includes the face, forehead, lips, scalp, neck, shoulder, arms, hands, thighs, legs, knees, feet, chest, breast, back, groin, buttocks, or any combination thereof.
97. The method according to embodiment 95 or 96, wherein the attribute or the characteristic of the skin region includes one or more signs of dermatological aging and/or environmental stress.
98. The method according to any one of embodiments 95-97, wherein the attribute or the characteristic of the skin region includes restoration, maintenance and/or improvement of any attribute or characteristic of a skin region include disappearance, reduction, and/or prevention of fine lines or wrinkles; disappearance, reduction, and/or prevention of skin sagging and/or skin sinking; disappearance, reduction, and/or prevention of skin thinning; disappearance, reduction, and/or prevention of skin dryness and/or itchiness; disappearance, reduction, and/or prevention of pigment spots and/or mottled skin; reduction of skin pore size; improvement in skin firmness; improvement in skin thickness, plumpness, and/or tautness; improvement in skin smoothness, suppleness and/or softness; improvement in skin tone, radiance, and/or clarity; improvement in procollagen, and/or collagen production; improvement in maintenance and remodeling of elastin; improvement in skin texture and/or promotion of retexturization; improvement in skin barrier repair and/or function; improvement in appearance of skin contours; restoration of skin luster and/or brightness; replenishment of essential nutrients and/or constituents in the skin; improvement of skin appearance decreased by aging and/or menopause; improvement in skin moisturization; increase in skin elasticity and/or resiliency; improvement of optical properties of skin by light diffraction or reflection; or any combination thereof.
99. A method to improve and/or support the appearance, beauty and/or health of a hair region in an individual, the method comprising topically applying a composition as defined in any one of embodiment 1-94, wherein application of the composition restores, maintains and/or improves an attribute or a characteristic of the hair region, thereby improving and/or supporting the appearance, beauty and/or health of the hair region.
100. The method according to embodiment 99, wherein the hair region includes scalp hair, facial hair, eyebrows, body hair, pubic hair, or any combination thereof.
101. The method according to embodiment 99 or 100, wherein the attribute or the characteristic of the skin region includes one or more signs of damage from environmental, physical or chemical exposure.
102. The method according to any one of embodiments 99-101, wherein the attribute or the characteristic of the skin region includes restoration, maintenance and/or improvement of any attribute or characteristic of a hair region, including improvement of. Non-limiting examples of restoration, maintenance and/or improvement of any attribute or characteristic of a hair region include disappearance, reduction, and/or prevention of rough, dry and/or brittle hair fibers; disappearance, reduction, and/or prevention of hair fiber breakage; disappearance, reduction, and/or prevention of hair fiber frayed or split ends; disappearance, reduction, and/or prevention of undesirable hair color change; disappearance, reduction, and/or prevention of loss of hair shine; disappearance, reduction, and/or prevention of loss of hair; disappearance, reduction, and/or prevention of disentangled hair; improvement in hair suppleness; improvement in hair luster; improvement in hair shine; improvement in hair manageability; improvement in the combing or brushing of hair; or any combination thereof.
103. The method according to any one of embodiments 95-102, wherein the individual is a mammal.
104. The method according to embodiment 103, wherein the mammal includes a human, a dog or a cat.
105. The method according to any one of embodiments 95-104, wherein the composition is applied by rubbing, pouring, sprinkling, or spraying.
106. The method according to any one of embodiments 95-104, wherein the composition is applied using a solid support.
107. The method according to embodiment 106, wherein the solid support is a wipe, a patch, a strip, a towelette, a towel, a mitt, a glove or a mask.
108. The method according to any one of embodiments 95-104, wherein the composition is applied using a delivery device.
109. The method according to embodiment 108, wherein the delivery device is an aerosol dispenser, a dropper, a pump spray, a trigger spray or a squeeze bottle.
110. The method according to any one of embodiments 95-109, wherein the composition is applied daily, every other day, every third of day, once a week, multiple times per week, once a month, multiple times per month, once a year, multiple times per year or as often as desired.
111. The method according to any one of embodiments 95-109, wherein the composition is applied multiple times per day.
112. The method according to embodiment 111, wherein the multiple times per day is twice a day, three times a day, four time a day, five times a day, six times a day or as often as desired.
113. A method to improve and/or support the appearance, beauty and/or health of a skin region in an individual, the method comprising topically applying a composition comprising hypochlorous acid or free available chlorine, wherein application of the composition restores, maintains and/or improves an attribute or a characteristic of the skin region, thereby improving and/or supporting the appearance, beauty and/or health of the skin region.
114. A composition comprising hypochlorous acid or free available chlorine, one or more silicones, one or more silicas and one or more silicate clays.
115. The composition according to embodiment 114, wherein the hypochlorous acid or free available chlorine is in an amount of 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition or at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition or at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition or about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01% by weight of the composition.
116. The composition according to embodiment 114 or 115, wherein the hypochlorous acid or free available chlorine is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.
117. The composition according to any one of embodiments 114-116, wherein the one or more silicones comprise one or more non-volatile silicones, one or more volatile silicones, one or more silicone surfactants, or any combination thereof.
118. The composition according to embodiment 117, wherein the one or more non-volatile silicones include one or more polysiloxanes, one or more polysiloxane copolymers, one or more polyalkyl siloxanes, one or more polyaryl siloxanes, one or more polyalkylaryl siloxanes, one or more polyether siloxanes, one or more poly(alkylarylsiloxane)-(ethersiloxane) copolymers, one or more polydimethylsiloxanes, one or more polydiphenylsiloxanes, one or more poly(dimethylsiloxane)-(diphenylsiloxane) copolymers, one or more phenyl silicones, one or more polysilicate siloxane copolymers, or any combination thereof.
119. The composition according to embodiment 117, wherein the one or more volatile silicones include one or more cyclomethicones.
120. The composition according to embodiment 117, wherein the one or more silicone surfactants include disodium PEG-12 dimethicone sulfosuccinate, PEG-12 dimethicone, PEG-10 dimethicone, PEG-8 dimethicone, PEG-7 dimethicone, PEG-7 dimethicone succinate, PEG-20/PPG-6 dimethicone, PEG-14/PPG-4 dimethicone, PEG-4/PPG-12 dimethicone, PEG-20/PPG-20 dimethicone, dimethylsiloxane and methyl(polyoxyethylene)siloxane copolymers, dimethylsiloxane and methyl(polyoxyethylene)siloxane and methyl(polyoxypropyl)siloxane copolymers, PEG-8 trisiloxane PEG-8/PPG-26 dimethicone, PEG-20/PPG-23 dimethicone, PEG-20/PPG-15 dimethicone, PEG-23/PPG-6 dimethicone, PEG-17 dimethicone, PPG-12 dimethicone, Quaternium-80, Quaternium 86, PEG-8 Distearmonium Chloride PG-Dimethicone, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-16/Glycidoxy Dimethicone Crosspolymer, Silicone Quaternium-17, Silicone Quaternium-18, Silicone Quaternium-19, Silicone Quaternium-20, Silicone Quaternium-21, Dimethicone Hydroxypropyl Trimonium Chloride, Hydroxyethyl Acetomonium PG-Dimethicone, Stearalkonium Dimethicone PEG-8 Phthalate, and Steardimonium Hydroxypropyl Panthenyl PEG-7 Dimethicone Phosphate Chloride.
121. The composition according to any one of embodiments 114-117, wherein the one or more silicones comprise one or more silicone amines, one or more anionic silicone, or both one or more silicone amines and one or more anionic silicone.
122. The composition according to embodiment 121, wherein the one or more silicone amines includes an amodimethicone, a trimethylsilylamodimethicone, a trimethylsilylamodimethicone, a phenyltrimethicone, an aminopropyl phenyltrimethicone, a trimethyloxysilane, a triethyoxysilane, a tearoxytrimethylsilane, an aminopropyl triethyoxysilane, an alkyl modified amino silicone, an amino silane, a polyethylene glycol based amodimethicone, a bis-isobutyl PEG-14/amodimethicone copolymer, a bis-isobutyl PEG-15/amodimethicone copolymer, a bis-isobutyl PEG/PPG-20/35/amodimethicone copolymer, or any combination thereof.
123. The composition according to embodiment 122, wherein the polyethylene glycol based amodimethicone includes a PEG-7 amodimethicone or a PEG-8 amodimethicone.
124. The composition according to embodiment 122, wherein the modified aminosiloxane includes a Trideceth-9 PG-amodimethicone.
125. The composition according to any one of embodiments 121-123, wherein the one or more anionic silicones includes a polydimethylsiloxane, a silicone carboxylate, a silicone phosphate, a silicone sulfate, a silicone sulfosuccinate, a silicone sulfonate, a siloxane, or any combination thereof.
126. The composition according to embodiment 125, wherein the polydimethylsiloxane includes a dimethicone, a vinyl dimethicone, a dimethicone/vinyl dimethicone copolymer, a dimethicone/vinyl dimethicone crosspolymer or a cyclodimethicone.
127. The composition according to embodiment 126, wherein the cyclodimethicone includes hexamethylcyclotrisiloxane, octamethylcyclotetrasiloxane, decamethylcyclopentasiloxane and dodecamethylcyclohexasiloxane.
128. The composition according to embodiment 125, wherein the silicone carboxylate includes a dimethicone PEG-7 phthalate or a dimethicone PEG-7 succinate.
129. The composition according to embodiment 125, wherein the silicone phosphate includes a dimethicone PEG-7 phosphate, a dimethicone PEG-8 phosphate, a dimethicone PEG-10 phosphate, a potassium dimethicone PEG-7 panthenyl phosphate, a dimethicone PEG/PPG-7/4 phosphate or a dimethicone PEG/PPG-12/4 phosphate.
130. The composition according to embodiment 125, wherein the siloxane includes a polysilicone-11, a polysilicone-15, a silica silylate, a silica dimethyl silylate, a trimethylsiloxysilicate, a trifluoropropyldimethyl/trimethylsiloxysilicate or an alkyldimethylsilyl polypropylsilsesquioxane.
131. The composition according to any one of embodiments 114-130, wherein the one or more silicones are an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, 10%, 15%, 20%, 25%, 30%, 35%, or 40% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, least 10%, least 15%, least 20%, least 25%, least 30%, least 35%, or least 40% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, most 10%, most 15%, most 20%, most 25%, most 30%, most 35%, or most 40% by weight of the composition or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.
132. The composition according to any one of embodiments 121-131, wherein the one or more silicone amines and the one or more anionic silicones are in a ratio of from about 1:20 to about 20:1, about 1:15 to about 15:1, about 1:10 to about 10:1, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, or about 1:1.
133. The composition according to any one of embodiments 114-132, wherein the one or more silicones are an amount of 0.001%, 0.0025%, 0.005%, 0.0075%, 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, or 10% by weight of the composition; or at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition; or at most 0.001%, at most 0.0025%, at most 0.005%, at most 0.0075%, at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition; or about 0.001% to about 0.005%, 0.001% to about 0.0075%, about 0.001% to about 0.01%, about 0.001% to about 0.05%, about 0.001% to about 0.075%, about 0.001% to about 0.1%, about 0.001% to about 0.25%, about 0.001% to about 0.5%, about 0.001% to about 0.75%, about 0.005% to about 0.0075%, about 0.005% to about 0.01%, about 0.005% to about 0.05%, about 0.005% to about 0.075%, about 0.005% to about 0.1%, about 0.005% to about 0.25%, about 0.005% to about 0.5%, about 0.005% to about 0.75%, about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.
134. The composition according to any one of embodiments 114-133, wherein the silicate clay is in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, or 10% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.
135. The composition according to any one of embodiments 1-94 or 114-134, or a method according to any one of embodiments 95-113, wherein a therapeutic or clinical effect is provided that reduces one or more symptoms associated with a skin or hair condition.
136. The composition or method according to embodiment 135, wherein the therapeutic or clinical effect comprises reduced itching, reduced pain, or increased healing of a skin abrasion.
137. The composition according to any one of embodiments 1-94 or 114-134, or a method according to any one of embodiments 95-113, wherein the composition does not include amodimethicone.
138. A kit comprising a pharmaceutical composition as defined in any one of embodiments 1-94 or 114-137.
139. The kit according to Embodiment 138, further comprising one or more delivery or application systems, and/or instructions, and/or a container.
140. A kit comprising a first component including hypochlorous acid or free available chloride and a second component including one or more silicones.
141. The kit according to embodiment 140, further comprising a third component including one or more rheology agents.
142. The kit according to Embodiment 140 or 141, further comprising one or more delivery or application systems, and/or instructions, and/or a container.
143. A method to improve and/or support the appearance, beauty and/or health of a skin region in an individual, the method comprising topically applying a first component including hypochlorous acid or free available chloride and a second component including one or more silicones, wherein application of the first and second components restores, maintains and/or improves an attribute or a characteristic of the skin region, thereby improving and/or supporting the appearance, beauty and/or health of the skin region.
144. The method according to embodiment 143, further comprising application of a third component including one or more rheology agents.
145. The method according to embodiment 143 or 144, wherein the skin region includes the face, forehead, lips, scalp, neck, shoulder, arms, hands, thighs, legs, knees, feet, chest, breast, back, groin, buttocks, or any combination thereof.
146. The method according to any one of embodiments 143-145, wherein the attribute or the characteristic of the skin region includes one or more signs of dermatological aging and/or environmental stress.
147. The method according to any one of embodiments 143-146, wherein the attribute or the characteristic of the skin region includes restoration, maintenance and/or improvement of any attribute or characteristic of a skin region include disappearance, reduction, and/or prevention of fine lines or wrinkles; disappearance, reduction, and/or prevention of skin sagging and/or skin sinking; disappearance, reduction, and/or prevention of skin thinning; disappearance, reduction, and/or prevention of skin dryness and/or itchiness; disappearance, reduction, and/or prevention of pigment spots and/or mottled skin; reduction of skin pore size; improvement in skin firmness; improvement in skin thickness, plumpness, and/or tautness; improvement in skin smoothness, suppleness and/or softness; improvement in skin tone, radiance, and/or clarity; improvement in procollagen, and/or collagen production; improvement in maintenance and remodeling of elastin; improvement in skin texture and/or promotion of retexturization; improvement in skin barrier repair and/or function; improvement in appearance of skin contours; restoration of skin luster and/or brightness; replenishment of essential nutrients and/or constituents in the skin; improvement of skin appearance decreased by aging and/or menopause; improvement in skin moisturization; increase in skin elasticity and/or resiliency; improvement of optical properties of skin by light diffraction or reflection; or any combination thereof.
148. A method to improve and/or support the appearance, beauty and/or health of a hair region in an individual, the method comprising topically applying a first component including hypochlorous acid or free available chloride and a second component including one or more silicones, wherein application of the first and second components restores, maintains and/or improves an attribute or a characteristic of the hair region, thereby improving and/or supporting the appearance, beauty and/or health of the hair region.
149 The method according to embodiment 148, further comprising application of a third component including one or more rheology agents.
150. The method according to embodiment 148 or 149, wherein the hair region includes scalp hair, facial hair, eyebrows, body hair, pubic hair, or any combination thereof.
151. The method according to any one of embodiments 148-150, wherein the attribute or the characteristic of the skin region includes one or more signs of damage from environmental, physical or chemical exposure.
152. The method according to any one of embodiments 148-151, wherein the attribute or the characteristic of the skin region includes restoration, maintenance and/or improvement of any attribute or characteristic of a hair region, including improvement of. Non-limiting examples of restoration, maintenance and/or improvement of any attribute or characteristic of a hair region include disappearance, reduction, and/or prevention of rough, dry and/or brittle hair fibers; disappearance, reduction, and/or prevention of hair fiber breakage; disappearance, reduction, and/or prevention of hair fiber frayed or split ends; disappearance, reduction, and/or prevention of undesirable hair color change; disappearance, reduction, and/or prevention of loss of hair shine; disappearance, reduction, and/or prevention of loss of hair; disappearance, reduction, and/or prevention of disentangled hair; improvement in hair suppleness; improvement in hair luster; improvement in hair shine; improvement in hair manageability; improvement in the combing or brushing of hair; or any combination thereof.
153. The method according to any one of embodiments 143-152, wherein the individual is a mammal.
154. The method according to embodiment 153, wherein the mammal includes a human, a dog or a cat.
155. The method according to any one of embodiments 143-154, wherein the composition is applied by rubbing, pouring, sprinkling, or spraying.
156. The method according to any one of embodiments 143-154, wherein the composition is applied using a solid support.
157. The method according to embodiment 156, wherein the solid support is a wipe, a patch, a strip, a towelette, a towel, a mitt, a glove or a mask.
158. The method according to any one of embodiments 143-154, wherein the composition is applied using a delivery device.
159. The method according to embodiment 158, wherein the delivery device is an aerosol dispenser, a dropper, a pump spray, a trigger spray or a squeeze bottle.
160. The method according to any one of embodiments 143-159, wherein the composition is applied daily, every other day, every third of day, once a week, multiple times per week, once a month, multiple times per month, once a year, multiple times per year or as often as desired.
161. The method according to any one of embodiments 143-159, wherein the composition is applied multiple times per day.
162. The method according to embodiment 161, wherein the multiple times per day is twice a day, three times a day, four time a day, five times a day, six times a day or as often as desired.
163. The kit according to any one of embodiments 139-142 or a method according to any one of embodiments 143-162, wherein the hypochlorous acid or free available chlorine is in an amount of 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition or at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition or at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition, or any combination thereof; or about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01% by weight of the composition.
164. The kit according to any one of embodiments 139-142 or a method according to any one of embodiments 143-163, wherein the hypo; chlorous acid or free available chlorine is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm, or any combination thereof; or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.
165. The kit according to any one of embodiments 139-142, 163 or 164, or a method according to any one of embodiments 143-164, wherein the one or more silicones comprise one or more non-volatile silicones, one or more volatile silicones, one or more silicone surfactants, or any combination thereof.
166. The kit or method according to embodiment 165, wherein the one or more non-volatile silicones include one or more polysiloxanes, one or more polysiloxane copolymers, one or more polyalkyl siloxanes, one or more polyaryl siloxanes, one or more polyalkylaryl siloxanes, one or more polyether siloxanes, one or more poly(alkylarylsiloxane)-(ethersiloxane) copolymers, one or more polydimethylsiloxanes, one or more polydiphenylsiloxanes, one or more poly(dimethylsiloxane)-(diphenylsiloxane) copolymers, one or more phenyl silicones, one or more polysilicate siloxane copolymers, or any combination thereof.
167. The kit or method according to embodiment 165, wherein the one or more volatile silicones include one or more cyclomethicones.
168. The kit or method according to embodiment 165, wherein the one or more silicone surfactants include disodium PEG-12 dimethicone sulfosuccinate, PEG-12 dimethicone, PEG-10 dimethicone, PEG-8 dimethicone, PEG-7 dimethicone, PEG-7 dimethicone succinate, PEG-20/PPG-6 dimethicone, PEG-14/PPG-4 dimethicone, PEG-4/PPG-12 dimethicone, PEG-20/PPG-20 dimethicone, dimethylsiloxane and methyl(polyoxyethylene)siloxane copolymers, dimethylsiloxane and methyl(polyoxyethylene)siloxane and methyl(polyoxypropyl)siloxane copolymers, PEG-8 trisiloxane PEG-8/PPG-26 dimethicone, PEG-20/PPG-23 dimethicone, PEG-20/PPG-15 dimethicone, PEG-23/PPG-6 dimethicone, PEG-17 dimethicone, PPG-12 dimethicone, Quaternium-80, Quaternium 86, PEG-8 Distearmonium Chloride PG-Dimethicone, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-16/Glycidoxy Dimethicone Crosspolymer, Silicone Quaternium-17, Silicone Quaternium-18, Silicone Quaternium-19, Silicone Quaternium-20, Silicone Quaternium-21, Dimethicone Hydroxypropyl Trimonium Chloride, Hydroxyethyl Acetomonium PG-Dimethicone, Stearalkonium Dimethicone PEG-8 Phthalate, and Steardimonium Hydroxypropyl Panthenyl PEG-7 Dimethicone Phosphate Chloride.
169. The kit according to any one of embodiments 139-142, or 163-168, or a method according to any one of embodiments 143-168, wherein the one or more silicones comprise one or more silicone amines, one or more anionic silicone, or both one or more silicone amines and one or more anionic silicone.
170. The kit or method according to embodiment 169, wherein the one or more silicone amines includes an amodimethicone, a trimethylsilylamodimethicone, a trimethylsilylamodimethicone, a phenyltrimethicone, an aminopropyl phenyltrimethicone, a trimethyloxysilane, a triethyoxysilane, a tearoxytrimethylsilane, an aminopropyl triethyoxysilane, an alkyl modified amino silicone, an amino silane, a polyethylene glycol based amodimethicone, a bis-isobutyl PEG-14/amodimethicone copolymer, a bis-isobutyl PEG-15/amodimethicone copolymer, a bis-isobutyl PEG/PPG-20/35/amodimethicone copolymer, or any combination thereof.
171. The kit or method according to embodiment 170, wherein the polyethylene glycol based amodimethicone includes a PEG-7 amodimethicone or a PEG-8 amodimethicone.
172. The kit or method according to embodiment 170, wherein the modified aminosiloxane includes a Trideceth-9 PG-amodimethicone.
173. The kit or method according to any one of embodiments 165-172, wherein the one or more anionic silicones includes a polydimethylsiloxane, a silicone carboxylate, a silicone phosphate, a silicone sulfate, a silicone sulfosuccinate, a silicone sulfonate, a siloxane, or any combination thereof.
174. The kit or method according to embodiment 173, wherein the polydimethylsiloxane includes a dimethicone, a vinyl dimethicone, a dimethicone/vinyl dimethicone copolymer, a dimethicone/vinyl dimethicone crosspolymer or a cyclodimethicone.
175. The kit or method according to embodiment 174, wherein the cyclodimethicone includes hexamethylcyclotrisiloxane, octamethylcyclotetrasiloxane, decamethylcyclopentasiloxane and dodecamethylcyclohexasiloxane.
176. The kit or method according to embodiment 173, wherein the silicone carboxylate includes a dimethicone PEG-7 phthalate or a dimethicone PEG-7 succinate.
177. The kit or method according to embodiment 173, wherein the silicone phosphate includes a dimethicone PEG-7 phosphate, a dimethicone PEG-8 phosphate, a dimethicone PEG-10 phosphate, a potassium dimethicone PEG-7 panthenyl phosphate, a dimethicone PEG/PPG-7/4 phosphate or a dimethicone PEG/PPG-12/4 phosphate.
178. The kit or method according to embodiment 173, wherein the siloxane includes a polysilicone-11, a polysilicone-15, a silica silylate, a silica dimethyl silylate, a trimethylsiloxysilicate, a trifluoropropyldimethyl/trimethylsiloxysilicate or an alkyldimethylsilyl polypropylsilsesquioxane.
179. The kit according to any one of embodiments 139-142, or 163-178, or a method according to any one of embodiments 143-178, wherein the one or more silicones are an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, 10%, 15%, 20%, 25%, 30%, 35%, or 40% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, least 10%, least 15%, least 20%, least 25%, least 30%, least 35%, or least 40% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, most 10%, most 15%, most 20%, most 25%, most 30%, most 35%, or most 40% by weight of the composition, or any combination thereof; or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.
180. The kit or method according to any one of embodiments 169-179, wherein the one or more silicone amines and the one or more anionic silicones are in a ratio of from about 1:20 to about 20:1, about 1:15 to about 15:1, about 1:10 to about 10:1, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, or about 1:1.
181. The kit according to any one of embodiments 141, 142, or 163-180, or a method according to any one of embodiments 144-147 or 149-180, wherein the one or more rheology agents include a polyurethane-based polymer, a polyether polyol-based polymer, a castor oil derivative, a silica, a hydrophilic clay, a steareth-100/PEG-136/HDI copolymer, or any combination thereof.
182. The kit or method according to embodiment 181, wherein the polyurethane-based polymer includes bis-lauryl cocaminopropylamine/HDI/PEG-100 copolymer (and) butylene glycol or bis-C₁₆₋₂₀isoalkoxy TMHDI/PEG-90 copolymer.
183. The kit or method according to embodiment 181, wherein the polyether polyol-based polymer includes PEG/PPG-450/50 trimethylolpropane dodecyl ether.
184. The kit or method according to embodiment 181, wherein the castor oil derivative includes trihydroxystearin.
185. The kit or method according to embodiment 181, wherein the hydrophilic clay includes a hectorite clay or a silicate clay.
186. The kit according to any one of embodiments 141, 142, or 163-185, or a method according to any one of embodiments 144-147 or 149-185, wherein the rheology agent is in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%, 9%, or 10% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition, or any combination thereof; or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compositions, methods and uses disclosed herein.

### Example 1

### Hair Smoothing Composition

This example illustrates how to formulate a hair smoothing composition as a spray. The components listed below were mixed at ambient temperature to ensure a uniform mixture. The pH of the mixture was adjusted to 5.7 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 0.464% | 0.464% | 0.464% | 0.464% | 0.464% | 0.464% | 0.464% | 0.464% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

This example illustrates how to formulate a hair smoothing composition as a spray. The components listed below were mixed at ambient temperature to ensure a uniform mixture. The pH of the mixture was adjusted to 5.7 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 0.927% | 0.927% | 0.927% | 0.927% | 0.927% | 0.927% | 0.927% | 0.927% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

This example illustrates how to formulate a hair smoothing composition as a spray. The components listed below were mixed at ambient temperature to ensure a uniform mixture. The pH of the mixture was adjusted to 5.3 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 0.464% | 0.464% | 0.464% | 0.464% | 0.464% | 0.464% | 0.464% | 0.464% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

This example illustrates how to formulate a hair smoothing composition as a spray. The components listed below were mixed at ambient temperature to ensure a uniform mixture. The pH of the mixture was adjusted to 5.3 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 0.927% | 0.927% | 0.927% | 0.927% | 0.927% | 0.927% | 0.927% | 0.927% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

### Example 2

### Hair Smoothing Composition

This example illustrates how to formulate a hair smoothing composition as a spray. The components listed below were mixed at ambient temperature to ensure a uniform mixture. The pH of the mixture was adjusted to 5.7 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.464% | 0.464% | 0.464% | 0.464% | 0.464% | 0.464% | 0.464% | 0.464% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

This example illustrates how to formulate a hair smoothing composition as a spray. The components listed below were mixed at ambient temperature to ensure a uniform mixture. The pH of the mixture was adjusted to 5.7 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.927% | 0.927% | 0.927% | 0.927% | 0.927% | 0.927% | 0.927% | 0.927% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

This example illustrates how to formulate a hair smoothing composition as a spray. The components listed below were mixed at ambient temperature to ensure a uniform mixture. The pH of the mixture was adjusted to 5.3 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.464% | 0.464% | 0.464% | 0.464% | 0.464% | 0.464% | 0.464% | 0.464% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

This example illustrates how to formulate a hair smoothing composition as a spray. The components listed below were mixed at ambient temperature to ensure a uniform mixture. The pH of the mixture was adjusted to 5.3 using sodium hydroxide.

Formulations for Hair Smoothing Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.927% | 0.927% | 0.927% | 0.927% | 0.927% | 0.927% | 0.927% | 0.927% |
| Phosphate | 0.029% | 0.029% | 0.029% | 0.029% | 0.029% | 0.029% | 0.029% | 0.029% |
| Water | q.s. | q.s. | q.s. | q.s. | q.s. | q.s. | q.s. | q.s. |

### Example 3

### Anti-Aging Composition

This example illustrates how to formulate an anti-aging composition as a gel. The components listed below were mixed at ambient temperature to ensure a uniform mixture. The pH of the mixture was adjusted to 7.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone/vinyl | 0.98% | 0.98% | 0.98% | 0.98% | 0.98% | 0.98% | 0.98% | 0.98% |
| dimethicone | | | | | | | | |
| crosspolymer, silica¹ | | | | | | | | |
| Silicate clay² | 1.37% | 1.37% | 1.37% | 1.37% | 1.37% | 1.37% | 1.37% | 1.37% |
| Phosphate | 0.044% | 0.044% | 0.044% | 0.044% | 0.044% | 0.044% | 0.044% | 0.044% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate an anti-aging composition as a gel. The components listed below were mixed at ambient temperature to ensure a uniform mixture. The pH of the mixture was adjusted to 7.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone/vinyl | 1.03% | 1.03% | 1.03% | 1.03% | 1.03% | 1.03% | 1.03% | 1.03% |
| dimethicone | | | | | | | | |
| crosspolymer, silica¹ | | | | | | | | |
| Silicate clay² | 1.37% | 1.37% | 1.37% | 1.37% | 1.37% | 1.37% | 1.37% | 1.37% |
| Phosphate | 0.044% | 0.044% | 0.044% | 0.044% | 0.044% | 0.044% | 0.044% | 0.044% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate an anti-aging composition as a gel. The components listed below were mixed at ambient temperature to ensure a uniform mixture. The pH of the mixture was adjusted to 8.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone/vinyl | 0.98% | 0.98% | 0.98% | 0.98% | 0.98% | 0.98% | 0.98% | 0.98% |
| dimethicone | | | | | | | | |
| crosspolymer, silica¹ | | | | | | | | |
| Silicate clay² | 1.44% | 1.44% | 1.44% | 1.44% | 1.44% | 1.44% | 1.44% | 1.44% |
| Phosphate | 0.053% | 0.053% | 0.053% | 0.053% | 0.053% | 0.053% | 0.053% | 0.053% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate an anti-aging composition as a gel. The components listed below were mixed at ambient temperature to ensure a uniform mixture. The pH of the mixture was adjusted to 8.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone/vinyl | 1.03% | 1.03% | 1.03% | 1.03% | 1.03% | 1.03% | 1.03% | 1.03% |
| dimethicone | | | | | | | | |
| crosspolymer, silica¹ | | | | | | | | |
| Silicate clay² | 1.44% | 1.44% | 1.44% | 1.44% | 1.44% | 1.44% | 1.44% | 1.44% |
| Phosphate | 0.053% | 0.053% | 0.053% | 0.053% | 0.053% | 0.053% | 0.053% | 0.053% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

### Example 4

### Anti-Aging Composition

This example illustrates how to formulate an anti-aging composition as a gel. The components listed below were mixed at ambient temperature to ensure a uniform mixture. The pH of the mixture was adjusted to 7.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.98% | 0.98% | 0.98% | 0.98% | 0.98% | 0.98% | 0.98% | 0.98% |
| Silicate clay¹ | 1.37% | 1.37% | 1.37% | 1.37% | 1.37% | 1.37% | 1.37% | 1.37% |
| Phosphate | 0.044% | 0.044% | 0.044% | 0.044% | 0.044% | 0.044% | 0.044% | 0.044% |
| Water | q.s. | q.s. | q.s. | q.s. | q.s. | q.s. | q.s. | q.s. |

¹LAPONITE ® XLG or LAPONITE ® XL21.

This example illustrates how to formulate an anti-aging composition as a gel. The components listed below were mixed at ambient temperature to ensure a uniform mixture. The pH of the mixture was adjusted to 7.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 1.03% | 1.03% | 1.03% | 1.03% | 1.03% | 1.03% | 1.03% | 1.03% |
| Silicate clay¹ | 1.37% | 1.37% | 1.37% | 1.37% | 1.37% | 1.37% | 1.37% | 1.37% |
| Phosphate | 0.044% | 0.044% | 0.044% | 0.044% | 0.044% | 0.044% | 0.044% | 0.044% |
| Water | q.s. | q.s. | q.s. | q.s. | q.s. | q.s. | q.s. | q.s. |

¹LAPONITE ® XLG or LAPONITE ® XL21.

This example illustrates how to formulate an anti-aging composition as a gel. The components listed below were mixed at ambient temperature to ensure a uniform mixture. The pH of the mixture was adjusted to 8.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.98% | 0.98% | 0.98% | 0.98% | 0.98% | 0.98% | 0.98% | 0.98% |
| Silicate clay¹ | 1.44% | 1.44% | 1.44% | 1.44% | 1.44% | 1.44% | 1.44% | 1.44% |
| Phosphate | 0.053% | 0.053% | 0.053% | 0.053% | 0.053% | 0.053% | 0.053% | 0.053% |
| Water | q.s. | q.s. | q.s. | q.s. | q.s. | q.s. | q.s. | q.s. |

¹LAPONITE ® XLG or LAPONITE ® XL21.

This example illustrates how to formulate an anti-aging composition as a gel. The components listed below were mixed at ambient temperature to ensure a uniform mixture. The pH of the mixture was adjusted to 8.2 using sodium hydroxide. The viscosity is about 10,500 mPa·s to about 14,000 mPa·s.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 1.03% | 1.03% | 1.03% | 1.03% | 1.03% | 1.03% | 1.03% | 1.03% |
| Silicate clay¹ | 1.44% | 1.44% | 1.44% | 1.44% | 1.44% | 1.44% | 1.44% | 1.44% |
| Phosphate | 0.053% | 0.053% | 0.053% | 0.053% | 0.053% | 0.053% | 0.053% | 0.053% |
| Water | q.s. | q.s. | q.s. | q.s. | q.s. | q.s. | q.s. | q.s. |

¹LAPONITE ® XLG or LAPONITE ® XL21.

### Example 5

### Anti-Aging Composition

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 4.8% | 4.8% | 4.8% | 4.8% | 4.8% | 4.8% | 4.8% | 4.8% |
| Phosphate | 0.043% | 0.043% | 0.043% | 0.043% | 0.043% | 0.043% | 0.043% | 0.043% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 1.12% | 1.12% | 1.12% | 1.12% | 1.12% | 1.12% | 1.12% | 1.12% |
| Phosphate | 0.043% | 0.043% | 0.043% | 0.043% | 0.043% | 0.043% | 0.043% | 0.043% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 0.93% | 0.93% | 0.93% | 0.93% | 0.93% | 0.93% | 0.93% | 0.93% |
| Phosphate | 0.043% | 0.043% | 0.043% | 0.043% | 0.043% | 0.043% | 0.043% | 0.043% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Amodimethicone | 0.46% | 0.46% | 0.46% | 0.46% | 0.46% | 0.46% | 0.46% | 0.46% |
| Phosphate | 0.043% | 0.043% | 0.043% | 0.043% | 0.043% | 0.043% | 0.043% | 0.043% |
| Water | q.s. | q.s. | q.s. | q.s. | q.s. | q.s. | q.s. | q.s. |

### Example 6

### Anti-Aging Composition

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 4.8% | 4.8% | 4.8% | 4.8% | 4.8% | 4.8% | 4.8% | 4.8% |
| Phosphate | 0.055% | 0.055% | 0.055% | 0.055% | 0.055% | 0.055% | 0.055% | 0.055% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 1.12% | 1.12% | 1.12% | 1.12% | 1.12% | 1.12% | 1.12% | 1.12% |
| Phosphate | 0.055% | 0.055% | 0.055% | 0.055% | 0.055% | 0.055% | 0.055% | 0.055% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to 6.9 using sodium hydroxide.

Formulations for Anti-Aging Composition

| | | | | | | | | |
|---|---|---|---|---|---|---|---|---|
| Hypochlorous acid | 0.001% | 0.0015% | 0.0025% | 0.0030% | 0.0035% | 0.0050% | 0.010% | 0.015% |
| Dimethicone | 1.13% | 1.13% | 1.13% | 1.13% | 1.13% | 1.13% | 1.13% | 1.13% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.93% | 0.93% | 0.93% | 0.93% | 0.93% | 0.93% | 0.93% | 0.93% |
| Phosphate | 0.055% | 0.055% | 0.055% | 0.055% | 0.055% | 0.055% | 0.055% | 0.055% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to 7.2 using sodium hydroxide.

Formulations for Anti-Aging Composition

| | | | | | | | | |
|---|---|---|---|---|---|---|---|---|
| Hypochlorous acid | 0.001% | 0.0015% | 0.0024% | 0.0029% | 0.0040% | 0.0050% | 0.010% | 0.015% |
| Dimethicone | 0.56% | 0.56% | 0.56% | 0.56% | 0.56% | 0.56% | 0.56% | 0.56% |
| Phosphate | 0.055% | 0.055% | 0.055% | 0.055% | 0.055% | 0.055% | 0.055% | 0.055% |
| Water | q.s. | q.s. | q.s. | q.s. | q.s. | q.s. | q.s. | q.s. |

This example illustrates how to formulate an anti-aging composition as a spray. The components listed below were mixed at ambient temperature to ensure a uniform mixture. The pH of the mixture was adjusted to about 8.6 using sodium hydroxide.

Formulations for Anti-Aging Composition

| | | | | | | | | |
|---|---|---|---|---|---|---|---|---|
| Hypochlorous acid | 10 ppm | 15 ppm | 20 ppm | 25 ppm | 30 ppm | 35 ppm | 40 ppm | 45 ppm |
| Dimethicone | 2.17% | 2.17% | 2.17% | 2.17% | 2.17% | 2.17% | 2.17% | 2.17% |
| Silicate clay² | 1.59% | 1.59% | 1.59% | 1.59% | 1.59% | 1.59% | 1.59% | 1.59% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

### Example 7

### Ant-Aging Composition

This example illustrates how to formulate an anti-aging composition as make-up. The components listed below were mixed at ambient temperature to ensure a uniform mixture. The pH of the mixture was adjusted to 8.6 using sodium hydroxide. The viscosity is about 10,500 mPa·s to about 14,000 mPa·s.

Formulations for Anti-Aging Make-up Composition

| | | | | | | | | |
|---|---|---|---|---|---|---|---|---|
| Hypochlorous acid | 0.001% | 0.0015% | 0.0025% | 0.003% | 0.0040% | 0.0050% | 0.010% | 0.015% |
| Dimethicone/vinyl | 1.05% | 1.05% | 1.05% | 1.05% | 1.05% | 1.05% | 1.05% | 1.05% |
| dimethicone | | | | | | | | |
| crosspolymer, silica¹ | | | | | | | | |
| Dimethicone | 1.08% | 1.08% | 1.08% | 1.08% | 1.08% | 1.08% | 1.08% | 1.08% |
| Silicate clay² | 1.86% | 1.86% | 1.86% | 1.86% | 1.86% | 1.86% | 1.86% | 1.86% |
| Phosphate | 0.054% | 0.054% | 0.054% | 0.054% | 0.054% | 0.054% | 0.054% | 0.054% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate a make-up composition. The components listed below were mixed at ambient temperature to ensure a uniform mixture. The pH of the mixture was adjusted to 8.7 using sodium hydroxide. The viscosity is about 10,500 mPa·s to about 14,000 mPa·s.

Formulations for Anti-Aging Make-up Composition

| | | | | | | | | |
|---|---|---|---|---|---|---|---|---|
| Hypochlorous acid | 0.001% | 0.0015% | 0.0025% | 0.003% | 0.0040% | 0.0050% | 0.010% | 0.015% |
| Dimethicone/vinyl | 1.05% | 1.05% | 1.05% | 1.05% | 1.05% | 1.05% | 1.05% | 1.05% |
| dimethicone | | | | | | | | |
| crosspolymer, silica¹ | | | | | | | | |
| Dimethicone | 1.08% | 1.08% | 1.08% | 1.08% | 1.08% | 1.08% | 1.08% | 1.08% |
| Silicate clay² | 1.86% | 1.86% | 1.86% | 1.86% | 1.86% | 1.86% | 1.86% | 1.86% |
| Phosphate | 0.054% | 0.054% | 0.054% | 0.054% | 0.054% | 0.054% | 0.054% | 0.054% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate a make-up composition. The components listed below were mixed at ambient temperature to ensure a uniform mixture. The pH of the mixture was adjusted to 8.6 using sodium hydroxide. The viscosity is about 10,500 mPa·s to about 14,000 mPa·s.

Formulations for Anti-Aging Make-up Composition

| | | | | | | | | |
|---|---|---|---|---|---|---|---|---|
| Hypochlorous acid | 0.001% | 0.0015% | 0.0025% | 0.003% | 0.0040% | 0.0050% | 0.010% | 0.015% |
| Dimethicone/vinyl | 1.09% | 1.09% | 1.09% | 1.09% | 1.09% | 1.09% | 1.09% | 1.09% |
| dimethicone | | | | | | | | |
| crosspolymer, silica¹ | | | | | | | | |
| Dimethicone | 1.13% | 1.13% | 1.13% | 1.13% | 1.13% | 1.13% | 1.13% | 1.13% |
| Silicate clay² | 1.94% | 1.94% | 1.94% | 1.94% | 1.94% | 1.94% | 1.94% | 1.94% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate a make-up composition. The components listed below were mixed at ambient temperature to ensure a uniform mixture. The pH of the mixture was adjusted to 8.7 using sodium hydroxide. The viscosity is about 13.000 mPa·s to about 15.500 mPa·s.

Formulations for Anti-Aging Make-up Composition

| | | | | | | | | |
|---|---|---|---|---|---|---|---|---|
| Hypochlorous acid | 0.001% | 0.0015% | 0.0025% | 0.003% | 0.0040% | 0.0050% | 0.010% | 0.015% |
| Dimethicone/vinyl | 0.25% | 0.25% | 0.25% | 0.25% | 0.25% | 0.25% | 0.25% | 0.25% |
| dimethicone | | | | | | | | |
| crosspolymer, silica¹ | | | | | | | | |
| Dimethicone | 0.88% | 0.88% | 0.88% | 0.88% | 0.88% | 0.88% | 0.88% | 0.88% |
| Silicate clay² | 2.11% | 2.11% | 2.11% | 2.11% | 2.11% | 2.11% | 2.11% | 2.11% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

### Example 8

### Ant-Aging Composition

This example illustrates how to formulate an anti-aging composition as gel. The components listed below were mixed at ambient temperature to ensure a uniform mixture. The pH of the mixture was adjusted to 7.5 using sodium hydroxide. The viscosity is about 500 mPa·s to about 1,500 mPa·s.

Formulations for Anti-Aging Composition

| | | | | | | | | |
|---|---|---|---|---|---|---|---|---|
| Hypochlorous acid | 0.0025% | 0.005% | 0.0075% | 0.0090% | 0.010% | 0.011% | 0.012% | 0.015% |
| Dimethicone/vinyl | 0.17% | 0.17% | 0.17% | 0.17% | 0.17% | 0.17% | 0.17% | 0.17% |
| dimethicone | | | | | | | | |
| crosspolymer, silica¹ | | | | | | | | |
| Dimethicone | 4.39% | 4.39% | 4.39% | 4.39% | 4.39% | 4.39% | 4.39% | 4.39% |
| Silicate clay² | 0.79% | 0.79% | 0.79% | 0.79% | 0.79% | 0.79% | 0.79% | 0.79% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate an anti-aging composition as gel. The components listed below were mixed at ambient temperature to ensure a uniform mixture. The pH of the mixture was adjusted to 8.0 using sodium hydroxide. The viscosity is about 2,500 mPa·s to about 4,500 mPa·s.

Formulations for Anti-Aging Composition

| | | | | | | | | |
|---|---|---|---|---|---|---|---|---|
| Hypochlorous acid | 0.0025% | 0.005% | 0.0075% | 0.0090% | 0.010% | 0.011% | 0.012% | 0.015% |
| Dimethicone/vinyl | 0.88% | 0.88% | 0.88% | 0.88% | 0.88% | 0.88% | 0.88% | 0.88% |
| dimethicone | | | | | | | | |
| crosspolymer, silica¹ | | | | | | | | |
| Dimethicone | 0.25% | 0.25% | 0.25% | 0.25% | 0.25% | 0.25% | 0.25% | 0.25% |
| Silicate clay² | 1.59% | 1.59% | 1.59% | 1.59% | 1.59% | 1.59% | 1.59% | 1.59% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate an anti-aging composition as gel. The components listed below were mixed at ambient temperature to ensure a uniform mixture. The pH of the mixture was adjusted to 7.9 using sodium hydroxide. The viscosity is about 2,500 mPa·s to about 4,500 mPa·s.

Formulations for Anti-Aging Composition

| | | | | | | | | |
|---|---|---|---|---|---|---|---|---|
| Hypochlorous acid | 0.005% | 0.010% | 0.015% | 0.017% | 0.018% | 0.019% | 0.02% | 0.025% |
| Dimethicone/vinyl | 0.88% | 0.88% | 0.88% | 0.88% | 0.88% | 0.88% | 0.88% | 0.88% |
| dimethicone | | | | | | | | |
| crosspolymer, silica¹ | | | | | | | | |
| Dimethicone | 0.44% | 0.44% | 0.44% | 0.44% | 0.44% | 0.44% | 0.44% | 0.44% |
| Silicate clay² | 1.59% | 1.59% | 1.59% | 1.59% | 1.59% | 1.59% | 1.59% | 1.59% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate an anti-aging composition as gel. The components listed below were mixed at ambient temperature to ensure a uniform mixture. The pH of the mixture was adjusted to 7.7 using sodium hydroxide. The viscosity is about 2,500 mPa·s to about 4,500 mPa·s.

Formulations for Anti-Aging Composition

| | | | | | | | | |
|---|---|---|---|---|---|---|---|---|
| Hypochlorous acid | 0.010% | 0.015% | 0.020% | 0.025% | 0.030% | 0.035% | 0.040% | 0.045% |
| Dimethicone/vinyl | 0.88% | 0.88% | 0.88% | 0.88% | 0.88% | 0.88% | 0.88% | 0.88% |
| dimethicone | | | | | | | | |
| crosspolymer, silica¹ | | | | | | | | |
| Dimethicone | 0.44% | 0.44% | 0.44% | 0.44% | 0.44% | 0.44% | 0.44% | 0.44% |
| Silicate clay² | 1.59% | 1.59% | 1.59% | 1.59% | 1.59% | 1.59% | 1.59% | 1.59% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

This example illustrates how to formulate an anti-aging composition as gel. The components listed below were mixed at ambient temperature to ensure a uniform mixture. The pH of the mixture was adjusted to about 8.6 using sodium hydroxide. The viscosity is about 17,500 mPa·s to about 21,500 mPa·s.

Formulations for Anti-Aging Composition

| | | | | | | | | |
|---|---|---|---|---|---|---|---|---|
| Hypochlorous acid | 10 ppm | 15 ppm | 20 ppm | 25 ppm | 30 ppm | 35 ppm | 40 ppm | 45 ppm |
| Dimethicone/vinyl | 1.09% | 1.09% | 1.09% | 1.09% | 1.09% | 1.09% | 1.09% | 1.09% |
| dimethicone | | | | | | | | |
| crosspolymer, silica¹ | | | | | | | | |
| Dimethicone | 1.13% | 1.13% | 1.13% | 1.13% | 1.13% | 1.13% | 1.13% | 1.13% |
| Silicate clay² | 1.94% | 1.94% | 1.94% | 1.94% | 1.94% | 1.94% | 1.94% | 1.94% |
| Phosphate | 0.056% | 0.056% | 0.056% | 0.056% | 0.056% | 0.056% | 0.056% | 0.056% |
| Water | q.s. | q.s. | q.s. | q.s. | q.s. | q.s. | q.s. | q.s. |

¹DOW CORNING ® 9701 Cosmetic Powder (contains 93% to 97% by weight dimethicone/vinyl dimethicone crosspolymer and 3% to 7% by weight silica).
²LAPONITE ® XLG or LAPONITE ® XL21.

### Example 9

### Observational Studies

This example summaries two different observational studies testing the efficacy of compositions disclosed herein using volunteer participants.

In one study, eight individuals tested a gel formulation comprising about 25 ppm to about 30 ppm hypochlorous acid or free available chlorine, about 0.6% to about 1.6% of a dimethicone, about 0.6% to about 1.6% of a dimethicone/vinyl dimethicone crosspolymer, and about 1.5% to about 2.5% of a silicate clay and having a pH of about 8.5 to about 8.7. Participants were instructed to apply the gel formulation a minimum of once daily for at least 7 days. Interviews with each individual indicated that 6 of 8 (75%) participants reported positive, noticeable differences when comparing overall skin feel (texture and smoothness) before and after use. Best results were reported after a minimum of 2 days of treatment.

In another study, three individuals tested a hair spray formulation on the coat of their pet dogs. The hair spray formulation comprised about 25 ppm to about 30 ppm hypochlorous acid or free available chlorine, about 1.75% to about 2.75% of a dimethicone, and about 1.1% to about 2.1% of a silicate clay and has a pH of about 8.5 to about 8.7. The pet owners were instructed to apply the spray formulation a minimum of once daily for at least 7 days on the coats of their dogs. Interviews with each participant indicated that 3 of 3 (100%) owners reported positive noticeable difference when comparing overall hair texture of their animals before and after use. Best results were reported immediately after the initial use.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A composition comprising hypochlorous acid or free available chlorine and one or more silicones.

2. The composition according to claim 1, wherein the one or more silicones comprise one or more silicone amines, one or more anionic silicone, or both one or more silicone amines and one or more anionic silicone

3. The composition according to claim 1, wherein the one or more anionic silicones includes a polydimethylsiloxane, a silicone carboxylate, a silicone phosphate, a silicone sulfate, a silicone sulfosuccinate, a silicone sulfonate, a siloxane, or any combination thereof.

4. The composition according to claim 3, wherein the polydimethylsiloxane includes a dimethicone, a vinyl dimethicone, a dimethicone/vinyl dimethicone copolymer, a dimethicone/vinyl dimethicone crosspolymer or a cyclodimethicone.

5. The composition according to claim 1, further comprising one or more rheology agents.

6. The composition according to claim 5, wherein the one or more rheology agents include a polyurethane-based polymer, a polyether polyol-based polymer, a castor oil derivative, a silica, a hydrophilic clay, a steareth-100/PEG-136/HDI copolymer, or any combination thereof.

7. The composition according to claim 6, wherein the hydrophilic clay includes a hectorite clay or a silicate clay.

8. The composition according to claim 1, further comprising one or more surfactants, a humectant, a preservative, an emulsifier, a fragrance, a chelating agent, an astringent, a shine control agent, a colorant agent, a soothing agent, a calming agent, or any combination thereof.

9. The composition according to claim 1, formulated as a liquid composition, a colloidal composition, a semi-solid composition, or a solid composition.

10. The composition according to claim 1, formulated as a liquid aerosol, a foam, an emulsion, a gel, a sol, or a solid sol.

11. The composition according to claim 1, formulated as a spray, a liquid aerosol, a wash, an aftershave, a perfume, a body mist, a lotion, a cream, a salve, a waxing composition, a mousse, a shampoo, a conditioner, an ointment, a soap, a toner, a foundation, a stick, a film forming product, a suspension, or an emollient.

12. The composition according to claim 1, having a viscosity of 6,500 mPa·s to 10,500 mPa·s.

13. The composition according to claim 1, having a viscosity of 17,500 mPa·s to 21,500 mPa·s.

14. The composition according to claim 5, comprising about 25 ppm to about 30 ppm hypochlorous acid or free available chlorine, about 0.6% to about 1.6% of a dimethicone, about 0.6% to about 1.6% of a dimethicone/vinyl dimethicone crosspolymer, about 0.018% to about 0.11% silica, and about 1.5% to about 2.5% of a silicate clay.

15. The composition according to claim 5, comprising about 25 ppm to about 30 ppm hypochlorous acid or free available chlorine, about 1.75% to about 2.75% of a dimethicone, and about 1.1% to about 2.1% of a silicate clay.

16. A kit comprising a pharmaceutical composition as defined in claim 1.

17. The kit according to claim 16, comprising a first component including hypochlorous acid or free available chloride and a second component including one or more silicones.

18. The kit according to claim 17, further comprising a third component including one or more rheology agents.

19. The kit according to claim 16, further comprising one or more delivery or application systems, and/or instructions, and/or a container.

20. A method to improve and/or support the appearance, beauty and/or health of a skin region and/or a hair region in an individual, the method comprising topically applying a composition as defined in claim 1, wherein application of the composition restores, maintains and/or improves an attribute or a characteristic of the skin region and/or the hair region, thereby improving and/or supporting the appearance, beauty and/or health of the skin region and/or the hair region.
